SEQUENCE LISTING (168 sequences)

SEQ ID NO: 1 - DNA, 27 bp, Artificial Sequence
  I-CeuI recognition sequence
taactataacggtcctaaggtagcgaa

SEQ ID NO: 2 - DNA, 27 bp, Artificial Sequence
  I-SceI recognition sequence
taactataacggtcctaaggtagcgaa

SEQ ID NO: 3 - DNA, 30 bp, Artificial Sequence
  PI-PspI recognition sequence
tggcaaacagctattatgggtattatgggt

SEQ ID NO: 4 - DNA, 37 bp, Artificial Sequence
  PI-SceI recognition sequence
atctatgtcgggtgcggagaaagaggtaatgaaatgg

SEQ ID NO: 5 - DNA, 24 bp, Artificial Sequence
  I-AniI recognition sequence
ttgaggaggtttctctgtaaataa

SEQ ID NO: 6 - DNA, 26 bp, Artificial Sequence
  I-CeuI recognition sequence
taactataacggtcctaaggtagcga

SEQ ID NO: 7 - DNA, 30 bp, Artificial Sequence
  I-ChuI recognition sequence
gaaggtttggcacctcgatgtcggctcatc

SEQ ID NO: 8 - DNA, 23 bp, Artificial Sequence
  I-CpaI recognition sequence
cgatcctaaggtagcgaaattca

SEQ ID NO: 9 - DNA, 20 bp, Artificial Sequence
  I-CpaII recognition sequence
cccggctaactctgtgccag

SEQ ID NO: 10 - DNA, 30 bp, Artificial Sequence
  I-CreI recognition sequence
ctgggttcaaaacgtcgtgagacagtttgg

SEQ ID NO: 11 - DNA, 30 bp, Artificial Sequence
  I-DmoI recognition sequence
atgccttgccgggtaagttccggcgcgcat

SEQ ID NO: 12 - DNA, 24 bp, Artificial Sequence
  H-DreI recognition sequence
caaaacgtcgtaagttccggcgcg

SEQ ID NO: 13 - DNA, 24 bp, Artificial Sequence
  I-HmuI recognition sequence
agtaatgagcctaacgctcagcaa

SEQ ID NO: 14 - DNA, 24 bp, Artificial Sequence
  I-HmuII recognition sequence
agtaatgagcctaacgctcaacaa

SEQ ID NO: 15 - DNA, 24 bp, Artificial Sequence
  I-LlaI recognition sequence
cacatccataaccatatcattttt

SEQ ID NO: 16 - DNA, 30 bp, Artificial Sequence; identical to SEQ ID NO: 10
  I-MsoI recognition sequence

SEQ ID NO: 17 - DNA, 30 bp, Artificial Sequence
  PI-PfuI recognition sequence
gaagatgggaggagggaccggactcaactt

SEQ ID NO: 18 - DNA, 15 bp, Artificial Sequence
  PI-PkoII recognition sequence
cagtactacggttac

SEQ ID NO: 19 - DNA, 25 bp, Artificial Sequence
  I-PorI recognition sequence
gcgagcccgtaagggtgtgtacggg

SEQ ID NO: 20 - DNA, 29 bp, Artificial Sequence
  I-PpoI recognition sequence
taactatgactctcttaaggtagccaaat

SEQ ID NO: 21 - DNA, 30 bp, Artificial Sequence; identical to SEQ ID NO: 3
  PI-PspI recognition sequence

SEQ ID NO: 22 - DNA, 28 bp, Artificial Sequence
  I-ScaI recognition sequence
tgtcacattgaggtgcactagttattac

SEQ ID NO: 23 - DNA, 30 bp, Artificial Sequence
  I-SceI recognition sequence
agttacgctagggataacagggtaatatag

SEQ ID NO: 24 - DNA, 39 bp, Artificial Sequence
  PI-SceI recognition sequence
atctatgtcgggtgcggagaaagaggtaatgaaatggca

SEQ ID NO: 25 - DNA, 29 bp, Artificial Sequence
  I-SceII recognition sequence
ttttgattctttggtcaccctgaagtata

SEQ ID NO: 26 - DNA, 29 bp, Artificial Sequence
  I-SecIII recognition sequence
attggaggttttggtaactatttattacc

SEQ ID NO: 27 - DNA, 29 bp, Artificial Sequence
  I-SceIV recognition sequence
tcttttctcttgattagccctaatctacg

SEQ ID NO: 28 - DNA, 24 bp, Artificial Sequence
  I-SceV recognition sequence
aataattttcttcttagtaatgcc

SEQ ID NO: 29 - DNA, 24 bp, Artificial Sequence
  I-SceVI recognition sequence
gttatttaatgttttagtagttgg

SEQ ID NO: 30 - DNA, 28 bp, Artificial Sequence
  I-SceVII recognition sequence
tgtcacattgaggtgcactagttattac

SEQ ID NO: 31 - DNA, 19 bp, Artificial Sequence
  I-Ssp6803I recognition sequence
gtcgggctcataacccgaa

SEQ ID NO: 32 - DNA, 24 bp, Artificial Sequence
  I-TevI recognition sequence
agtggtatcaacgctcagtagatg

SEQ ID NO: 33 - DNA, 30 bp, Artificial Sequence
  I-TevII recognition sequence
gcttatgagtatgaagtgaacacgttattc

SEQ ID NO: 34 - DNA, 30 bp, Artificial Sequence
  I-TevIII recognition sequence
tatgtatcttttgcgtgtacctttaacttc

SEQ ID NO: 35 - DNA, 22 bp, Artificial Sequence
  PI-TliI recognition sequence
  variation 6,12: /note=“Wherein n is A, C, G or T”
taygcngayacngacggyttyt

SEQ ID NO: 36 - DNA, 30 bp, Artificial Sequence
  PI-TliII recognition sequence
aaattgcttgcaaacagctattacggctat

SEQ ID NO: 37 - DNA, 19 bp, Artificial Sequence
  I-Tsp061I recognition sequence
cttcagtatgccccgaaac

SEQ ID NO: 38 - DNA, 24 bp, Artificial Sequence
  I-Vdi141I recognition sequence
cctgactctcttaaggtagccaaa

SEQ ID NO: 39 - DNA, 21 bp, Artificial Sequence
  BneMS4ORFIP recognition sequence
aacgctcagcaattcccacgt

SEQ ID NO: 40 - DNA, 24 bp, Artificial Sequence
  F-CphI recognition sequence
gcacgagcgcaacgctcacaactt

SEQ ID NO: 41 - DNA, 40 bp, Artificial Sequence
  F-EcoT3I recognition sequence
gtacatggcatgggtacatgatgaaatccaagtaggctgc

SEQ ID NO: 42 - DNA, 30 bp, Artificial Sequence
  F-EcoT5I recognition sequence
tggcgacgaaaaccgcttggaaagtggctg

SEQ ID NO: 43 - DNA, 30 bp, Artificial Sequence
  F-EcoT5II recognition sequence
acctaccattaacggagtcaaaggccattg

SEQ ID NO: 44 - DNA, 30 bp, Artificial Sequence
  F-EcoT5IV recognition sequence
taggtactggacttaaaattcaggttttgt

SEQ ID NO: 45 - DNA, 20 bp, Artificial Sequence
  F-PhiU5I recognition sequence
aataacctgaagtatcaatc

SEQ ID NO: 46 - DNA, 24 bp, Artificial Sequence
  F-SceI recognition sequence
gatgctgtaggcataggcttggtt

SEQ ID NO: 47 - DNA, 20 bp, Artificial Sequence
  F-SceII recognition sequence
ctttccgcaacagtaaaatt

SEQ ID NO: 48 - DNA, 38 bp, Artificial Sequence
  F-TevI recognition sequence
  variation 25..38: /note=“Wherein n is A, C, G or T”
gaaacacaagaaatgtttagtaaannnnnnnnnnnnnn

SEQ ID NO: 49 - DNA, 29 bp, Artificial Sequence
  F-TevII recognition sequence
tttaatcctcgcttcagatatggcaactg

SEQ ID NO: 50 - DNA, 19 bp, Artificial Sequence
  F-TevIII recognition sequence
agaagaacatgtggtattg

SEQ ID NO: 51 - DNA, 35 bp, Artificial Sequence
  F-TevIV recognition sequence
tataggttcgaatcctatcacgtccgccaaatacg

SEQ ID NO: 52 - DNA, 24 bp, Artificial Sequence
  H-DreI recognition sequence
caaaacgtcgtaagttccggcgcg

SEQ ID NO: 53 - DNA, 26 bp, Artificial Sequence
  I-AabMI recognition sequence
caggtaccctttaaacctactaaccc

SEQ ID NO: 54 - DNA, 20 bp, Artificial Sequence
  I-AchMI recognition sequence
tggggaggtttttcagtatc

SEQ ID NO: 55 - DNA, 24 bp, Artificial Sequence
  I-AniI recognition sequence
ttgaggaggtttctctgtaaataa

SEQ ID NO: 56 - DNA, 20 bp, Artificial Sequence
  I-ApeKI recognition sequence
gcaaggctgaaacttaaagg

SEQ ID NO: 57 - DNA, 22 bp, Artificial Sequence
  I-BanI recognition sequence
attgaattcctggataccaaaa

SEQ ID NO: 58 - DNA, 21 bp, Artificial Sequence
  I-BasI recognition sequence
aacgctcagcaattcccacgt

SEQ ID NO: 59 - DNA, 28 bp, Artificial Sequence
  I-BmoI recognition sequence
gagtaagagcccgtagtaatgacatggc

SEQ ID NO: 60 - DNA, 39 bp, Artificial Sequence
  I-Bth0305I recognition sequence
atgtaaactcacgcttcggtgatccaaacgtgacaactg

SEQ ID NO: 61 - DNA, 23 bp, Artificial Sequence
  I-BthII recognition sequence
attatccgtgatgagtcaattca

SEQ ID NO: 62 - DNA, 28 bp, Artificial Sequence
  I-BthORFAP recognition sequence
gagtaagagcccgtagtaatgacatggc

SEQ ID NO: 63 - DNA, 27 bp, Artificial Sequence
  I-CeuI recognition sequence
taactataacggtcctaaggtagcgaa

SEQ ID NO: 64 - DNA, 30 bp, Artificial Sequence; identical to SEQ ID NO: 7
  I-ChuI recognition sequence

SEQ ID NO: 65 - DNA, 18 bp, Artificial Sequence
  I-CmoeI recognition sequence
tcgtagcagctcacggtt

SEQ ID NO: 66 - DNA, 23 bp, Artificial Sequence
  I-CpaI recognition sequence
cgatcctaaggtagcgaaattca

SEQ ID NO: 67 - DNA, 20 bp, Artificial Sequence
  I-CpaII recognition sequence
cccggctaactctgtgccag

SEQ ID NO: 68 - DNA, 28 bp, Artificial Sequence
  I-CpaMI recognition sequence
cctagcccacaatattaaggccatccca

SEQ ID NO: 69 - DNA, 30 bp, Artificial Sequence; identical to SEQ ID NO: 10
  I-CreI recognition sequence

SEQ ID NO: 70 - DNA, 17 bp, Artificial Sequence
  I-CreII recognition sequence
tgtagctgctcatggtt

SEQ ID NO: 71 - DNA, 30 bp, Artificial Sequence
  I-CsmI recognition sequence
gtactagcatggggtcaaatgtctttctgg

SEQ ID NO: 72 - DNA, 30 bp, Artificial Sequence; identical to SEQ ID NO: 10
  I-CvuI recognition sequence

SEQ ID NO: 73 - DNA, 24 bp, Artificial Sequence
  I-DdiI recognition sequence
ttttttggtcatccagaagtatat

SEQ ID NO: 74 - DNA, 30 bp, Artificial Sequence; identical to SEQ ID NO: 11
  I-DmoI recognition sequence

SEQ ID NO: 75 - DNA, 27 bp, Artificial Sequence
  I-GpeMI recognition sequence
cctttccgcttattcaaccctttaccc

SEQ ID NO: 76 - DNA, 22 bp, Artificial Sequence
  I-GpiI recognition sequence
ttttcctgtatatgacttaaat

SEQ ID NO: 77 - DNA, 22 bp, Artificial Sequence
  I-GzeI recognition sequence
gcccctcataacccgtatcaag

SEQ ID NO: 78 - DNA, 27 bp, Artificial Sequence
  I-GzeII recognition sequence
cctttgtaccaatatggtacccatccc

SEQ ID NO: 79 - DNA, 24 bp, Artificial Sequence
  I-HjeMI recognition sequence
tgaggaggtttctctgtaaattaa

SEQ ID NO: 80 - DNA, 21 bp, Artificial Sequence
  I-HmuI recognition sequence
aacgctcagcaattcccacgt

SEQ ID NO: 81 - DNA, 28 bp, Artificial Sequence
  I-HmuII recognition sequence
attattccagtataactttgagattaag

SEQ ID NO: 82 - DNA, 24 bp, Artificial Sequence
  I-LlaI recognition sequence
cacatccataaccatatcattttt

SEQ ID NO: 83 - DNA, 22 bp, Artificial Sequence
  I-LtrI recognition sequence
tatctaaacgtcgtataggagc

SEQ ID NO: 84 - DNA, 26 bp, Artificial Sequence
  I-LtrWI recognition sequence
agtagtgaagtatgttatttaattcg

SEQ ID NO: 85 - DNA, 22 bp, Artificial Sequence
  I-MpeMI recognition sequence
tagataaccataagtggctaat

SEQ ID NO: 86 - DNA, 30 bp, Artificial Sequence; identical to SEQ ID NO: 10
  I-MsoI recognition sequence

SEQ ID NO: 87 - DNA, 21 bp, Artificial Sequence
  I-NanI recognition sequence
aagtctggtgccagcacccgc

SEQ ID NO: 88 - DNA, 24 bp, Artificial Sequence
  I-NfiI recognition sequence
ttgaggaggtttccctgtaaataa

SEQ ID NO: 89 - DNA, 21 bp, Artificial Sequence
  I-NitI recognition sequence
aagtctggtgccagcacccgc

SEQ ID NO: 90 - DNA, 21 bp, Artificial Sequence
  I-NjaI recognition sequence
aagtctggtgccagcacccgc

SEQ ID NO: 91 - DNA, 21 bp, Artificial Sequence
  I-OmiII recognition sequence
gtagtgaagtatggttattta

SEQ ID NO: 92 - DNA, 14 bp, Artificial Sequence
  I-OnuI recognition sequence
ggttgaataagtgg

SEQ ID NO: 93 - DNA, 30 bp, Artificial Sequence; identical to SEQ ID NO: 10
  I-PakI recognition sequence

SEQ ID NO: 94 - DNA, 22 bp, Artificial Sequence
  I-PanMI recognition sequence
gctcctcataatccttatcaag

SEQ ID NO: 95 - DNA, 29 bp, Artificial Sequence
  I-PfoP3I recognition sequence
gactatcatcatctaagcctaacatccaa

SEQ ID NO: 96 - DNA, 24 bp, Artificial Sequence
  I-PnoMI recognition sequence
tgaggtggtttctctgtaaattaa

SEQ ID NO: 97 - DNA, 19 bp, Artificial Sequence
  I-PogTE7I recognition sequence
cttcagtatgccccgaaac

SEQ ID NO: 98 - DNA, 25 bp, Artificial Sequence
  I-PorI recognition sequence
gcgagcccgtaagggtgtgtacggg

SEQ ID NO: 99 - DNA, 29 bp, Artificial Sequence
  I-PpoI recognition sequence
taactatgactctcttaaggtagccaaat

SEQ ID NO: 100 - DNA, 28 bp, Artificial Sequence
  I-ScaI recognition sequence
tgtcacattgaggtgcactagttattac

SEQ ID NO: 101 - DNA, 18 bp, Artificial Sequence
  I-SceI recognition sequence
tagggataacagggtaat

SEQ ID NO: 102 - DNA, 29 bp, Artificial Sequence
  I-SceII recognition sequence
ttttgattctttggtcaccctgaagtata

SEQ ID NO: 103 - DNA, 29 bp, Artificial Sequence
  I-SceIII recognition sequence
attggaggttttggtaactatttattacc

SEQ ID NO: 104 - DNA, 29 bp, Artificial Sequence
  I-SceIV recognition sequence
tcttttctcttgattagccctaatctacg

SEQ ID NO: 105 - DNA, 24 bp, Artificial Sequence
  I-SceV recognition sequence
aataattttcttcttagtaatgcc

SEQ ID NO: 106 - DNA, 24 bp, Artificial Sequence
  I-SceVI recognition sequence
gttatttaatgttttagtagttgg

SEQ ID NO: 107 - DNA, 28 bp, Artificial Sequence
  I-SceVII recognition sequence
tgtcacattgaggtgcactagttattac

SEQ ID NO: 108 - DNA, 25 bp, Artificial Sequence
  I-SmaMI recognition sequence
ggtatcctccattatcaggtgtacg

SEQ ID NO: 109 - DNA, 25 bp, Artificial Sequence
  I-SpomI recognition sequence
gtggttggacggtatatccaccact

SEQ ID NO: 110 - DNA, 22 bp, Artificial Sequence
  I-SscMI recognition sequence
aggtaccctttaaacctattaa

SEQ ID NO: 111 - DNA, 19 bp, Artificial Sequence
  I-Ssp6803I recognition sequence
gtcgggctcataacccgaa

SEQ ID NO: 112 - DNA, 24 bp, Artificial Sequence
  I-TevI recognition sequence
agtggtatcaacgctcagtagatg

SEQ ID NO: 113 - DNA, 30 bp, Artificial Sequence; identical to SEQ ID NO: 33
  I-TevII recognition sequence

SEQ ID NO: 114 - DNA, 30 bp, Artificial Sequence; identical to SEQ ID NO: 34
  I-TevIII recognition sequence

SEQ ID NO: 115 - DNA, 40 bp, Artificial Sequence
  I-TslI recognition sequence
gtacatggcgtgggtgcatgatgaaatccaagtaggttgc

SEQ ID NO: 116 - DNA, 40 bp, Artificial Sequence
  I-TslWI recognition sequence
gtacatggcgtgggtgcatgatgagattcaagtaggttgc

SEQ ID NO: 117 - DNA, 19 bp, Artificial Sequence
  I-Tsp061I recognition sequence
cttcagtatgccccgaaac

SEQ ID NO: 118 - DNA, 20 bp, Artificial Sequence
  I-TwoI recognition sequence
tcttgcacctacacaatcca

SEQ ID NO: 119 - DNA, 24 bp, Artificial Sequence
  I-Vdi141I recognition sequence
cctgactctcttaaggtagccaaa

SEQ ID NO: 120 - DNA, 39 bp, Artificial Sequence
  PI-AvaI recognition sequence
gttgcaggcaatatccggcgtagtgccggaatgcgtcag

SEQ ID NO: 121 - DNA, 33 bp, Artificial Sequence
  PI-BciPI recognition sequence
tgggaaaaagccagtgggttcgaagagtctatg

SEQ ID NO: 122 - DNA, 30 bp, Artificial Sequence
  PI-HvoWI recognition sequence
gtggcctacggcgacaccgacagcgtcatg

SEQ ID NO: 123 - DNA, 22 bp, Artificial Sequence
  PI-MgaI recognition sequence
cgtagctgcccagtatgagtca

SEQ ID NO: 124 - DNA, 50 bp, Artificial Sequence
  PI-MleSI recognition sequence
  variation 38..50: /note=“Wherein n is A, C, G or T”
agaagattggggtgatgatgttcgggtctcccgaaacnnnnnnnnnnnnn

SEQ ID NO: 125 - DNA, 29 bp, Artificial Sequence
  PI-MtuI recognition sequence
aacgcggtcggcaaccgcacccgggtcac

SEQ ID NO: 126 - DNA, 22 bp, Artificial Sequence
  PI-PabI recognition sequence
gggggcagccagtggtcccgtt

SEQ ID NO: 127 - DNA, 23 bp, Artificial Sequence
  PI-PabII recognition sequence
acccctgtggagaggagcccctc

SEQ ID NO: 128 - DNA, 30 bp, Artificial Sequence; identical to SEQ ID NO: 17
  PI-PfuI recognition sequence

SEQ ID NO: 129 - DNA, 29 bp, Artificial Sequence
  PI-PfuII recognition sequence
acgaatccatgtggagaagagcctctata

SEQ ID NO: 130 - DNA, 19 bp, Artificial Sequence
  PI-PkoI recognition sequence
gattttagatccctgtacc

SEQ ID NO: 131 - DNA, 15 bp, Artificial Sequence
  PI-PkoII recognition sequence
cagtactacggttac

SEQ ID NO: 132 - DNA, 30 bp, Artificial Sequence; identical to SEQ ID NO: 3
  PI-PspI recognition sequence

SEQ ID NO: 133 - DNA, 30 bp, Artificial Sequence
  PI-ScaI recognition sequence
taagtcgggtgcggagaaagaggaaaagag

SEQ ID NO: 134 - DNA, 37 bp, Artificial Sequence; identical to SEQ ID NO: 4
  PI-SceI recognition sequence

SEQ ID NO: 135 - DNA, 26 bp, Artificial Sequence
  PI-TfuI recognition sequence
tagattttaggtcgctatatccttcc

SEQ ID NO: 136 - DNA, 22 bp, Artificial Sequence
  PI-TfuII recognition sequence
  variation 6,12: /note=“Wherein n is A, C, G or T”
taygcngayacngacggyttyt

SEQ ID NO: 137 - DNA, 22 bp, Artificial Sequence
  PI-ThyI recognition sequence
  variation 6,12: /note=“Wherein n is A, C, G or T”
taygcngayacngacggyttyt

SEQ ID NO: 138 - DNA, 22 bp, Artificial Sequence
  PI-TliI recognition sequence
  variation 6,12: /note=“Wherein n is A, C, G or T”
taygcngayacngacggyttyt

SEQ ID NO: 139 - DNA, 30 bp, Artificial Sequence; identical to SEQ ID NO: 36
  PI-TliII recognition sequence

SEQ ID NO: 140 - DNA, 19 bp, Artificial Sequence
  PI-TmaI recognition sequence
gaacagttactacggctac

SEQ ID NO: 141 - DNA, 19 bp, Artificial Sequence
  PI-TmaKI recognition sequence
gaacagttactacggctac

SEQ ID NO: 142 - DNA, 30 bp, Artificial Sequence
  PI-ZbaI recognition sequence
tacgttggttgtggtgaaagaggaaaagag

SEQ ID NO: 143 - DNA, 999 bp, Artificial Sequence
  Synthetic carrier gene sequence
cagcgttcgctataactataacggtcctaaggtagcgaaatgcaagagcaataccgcccggaagagatagaatccaaagtacagcttcatagggataacagggtaatttgggatgagaagcgcacatttgaagtaaccgaagacgagagcaaagagataactataacggtcctaaggtagcgaaagtattactgcctgtctatgcttccctatccttctggtcgactacacatgtagggataacagggtaatggccacgtacgtaactacaccatcggtgacgtgatcgcccgctaccagcgtaactataacggtcctaaggtagcgaatatgctgggcaaaaacgtcctgcagccgatcggctgggacgcgtttggtctagggataacagggtaattgcctgcggaaggcgcggcggtgaaaaacaacaccgctccggcaccgtggtaactataacggtcctaaggtagcgaaacgtacgacaacatcgcgtatatgaaaaaccagctcaaaatgctgggctttagggataacagggtaattggttatgactggagccgcgagctggcaacctgtacgccggaatactacctaactataacggtcctaaggtagcgaagttgggaacagaaattcttcaccgagctgtataaaaaaggcctggtatattagggataacagggtaataagaagacttctgcggtcaactggtgcccgaacgaccagaccgtactggctaactataacggtcctaaggtagcgaagaacgaacaagttatcgacggctgctgctggcgctgcgataccaaagttgtagggataacagggtaataacgtaaagagatcccgcagtggtttatcaaaatcactgcttacgctgactaactataacggtcctaaggtagcgaattgcagctcaacgatctggataaactggatcactggccagacaccgttaatagggataacagggtaatcgaattcgtctgcgacacgtag

SEQ ID NO: 144 - RNA, 1023 nt, Artificial Sequence
  Corresponding RNA transcript 1
  variation join(2..6,1018..1023): /note=“Wherein n is A, C, G or U”
gnnnnncagcguucgcuacagcguucgcuauaacuauaacgguccuaagguagcgaaaugcaagagcaauaccgcccggaagagauagaauccaaaguacagcuucauagggauaacaggguaauuugggaugagaagcgcacauuugaaguaaccgaagacgagagcaaagagauaacuauaacgguccuaagguagcgaaaguauuacugccugucuaugcuucccuauccuucuggucgacuacacauguagggauaacaggguaauggccacguacguaacuacaccaucggugacgugaucgcccgcuaccagcguaacuauaacgguccuaagguagcgaauaugcugggcaaaaacguccugcagccgaucggcugggacgcguuuggucuagggauaacaggguaauugccugcggaaggcgcggcggugaaaaacaacaccgcuccggcaccgugguaacuauaacgguccuaagguagcgaaacguacgacaacaucgcguauaugaaaaaccagcucaaaaugcugggcuuuagggauaacaggguaauugguuaugacuggagccgcgagcuggcaaccuguacgccggaauacuaccuaacuauaacgguccuaagguagcgaaguugggaacagaaauucuucaccgagcuguauaaaaaaggccugguauauuagggauaacaggguaauaagaagacuucugcggucaacuggugcccgaacgaccagaccguacuggcuaacuauaacgguccuaagguagcgaagaacgaacaaguuaucgacggcugcugcuggcgcugcgauaccaaaguuguagggauaacaggguaauaacguaaagagaucccgcagugguuuaucaaaaucacugcuuacgcugacuaacuauaacgguccuaagguagcgaauugcagcucaacgaucuggauaaacuggaucacuggccagacaccguuaauagggauaacaggguaaucgaauucgucugcgacacguagnnnnnn

SEQ ID NO: 145 - RNA, 1023 nt, Artificial Sequence; identical to SEQ ID NO: 144
  Corresponding RNA transcript 2
  variation join(2..6,1018..1023): /note=“Wherein n is A, C, G or U”

SEQ ID NO: 146 - RNA, 878 nt, Artificial Sequence
  Corresponding RNA transcript 3
  variation join(2..6,873..878): /note=“Wherein n is A, C, G or U”
gnnnnncagcguucgcuacagcguucgcuauaacuauaacgguccuaagguagcgaaaugcaagagcaauaccgcccggaagagauagaauccaaaguacagcuucauagggauaacaggguaauuugggaugagaagcgcacauuugaaguaaccgaagacgagagcaaagagauaacuauaacgguccuaagguagcgaaaguauuacugccugucuaugcuucccuauccuucuggucgacuacacauguagggauaacaggguaauggccacguacguaacuacaccaucggugacgugaucgcccgcuaccagcguaacuauaacgguccuaagguagcgaauaugcugggcaaaaacguccugcagccgaucggcugggacgcguuuggucuagggauaacaggguaauugccugcggaaggcgcggcggugaaaaacaacaccgcuccggcaccgugguaacuauaacgguccuaagguagcgaaacguacgacaacaucgcguauaugaaaaaccagcucaaaaugcugggcuuuagggauaacaggguaauugguuaugacuggagccgcgagcuggcaaccuguacgccggaauacuaccuaacuauaacgguccuaagguagcgaaguugggaacagaaauucuucaccgagcuguauaaaaaaggccugguauauuagggauaacaggguaauaagaagacuucugcggucaacuggugcccgaacgaccagaccguacuggcuaacuauaacgguccuaagguagcgaagaacgaacaaguuaucgacggcugcugcuggcgcugcgauaccaaaguuguagggauaacaggguaauaacguaaagagaucccgcagugnnnnnn

SEQ ID NO: 147 - RNA, 810 nt, Artificial Sequence
  Corresponding RNA transcript 4
  variation join(2..6,805..810): /note=“Wherein n is A, C, G or U”
gnnnnncagcguucgcuacagcguucgcuauaacuauaacgguccuaagguagcgaaaugcaagagcaauaccgcccggaagagauagaauccaaaguacagcuucauagggauaacaggguaauuugggaugagaagcgcacauuugaaguaaccgaagacgagagcaaagagauaacuauaacgguccuaagguagcgaaaguauuacugccugucuaugcuucccuauccuucuggucgacuacacauguagggauaacaggguaauggccacguacguaacuacaccaucggugacgugaucgcccgcuaccagcguaacuauaacgguccuaagguagcgaauaugcugggcaaaaacguccugcagccgaucggcugggacgcguuuggucuagggauaacaggguaauugccugcggaaggcgcggcggugaaaaacaacaccgcuccggcaccgugguaacuauaacgguccuaagguagcgaaacguacgacaacaucgcguauaugaaaaaccagcucaaaaugcugggcuuuagggauaacaggguaauugguuaugacuggagccgcgagcuggcaaccuguacgccggaauacuaccuaacuauaacgguccuaagguagcgaaguugggaacagaaauucuucaccgagcuguauaaaaaaggccugguauauuagggauaacaggguaauaagaagacuucugcggucaacuggugcccgaacgaccagaccguacuggcuaacuauaacgguccuaagguagcgaagaacgaacaaguuaucgacggcnnnnnn

SEQ ID NO: 148 - RNA, 733 nt, Artificial Sequence
  Corresponding RNA transcript 5
  variation join(2..6,728..733): /note=“Wherein n is A, C, G or U”
gnnnnncagcguucgcuacagcguucgcuauaacuauaacgguccuaagguagcgaaaugcaagagcaauaccgcccggaagagauagaauccaaaguacagcuucauagggauaacaggguaauuugggaugagaagcgcacauuugaaguaaccgaagacgagagcaaagagauaacuauaacgguccuaagguagcgaaaguauuacugccugucuaugcuucccuauccuucuggucgacuacacauguagggauaacaggguaauggccacguacguaacuacaccaucggugacgugaucgcccgcuaccagcguaacuauaacgguccuaagguagcgaauaugcugggcaaaaacguccugcagccgaucggcugggacgcguuuggucuagggauaacaggguaauugccugcggaaggcgcggcggugaaaaacaacaccgcuccggcaccgugguaacuauaacgguccuaagguagcgaaacguacgacaacaucgcguauaugaaaaaccagcucaaaaugcugggcuuuagggauaacaggguaauugguuaugacuggagccgcgagcuggcaaccuguacgccggaauacuaccuaacuauaacgguccuaagguagcgaaguugggaacagaaauucuucaccgagcuguauaaaaaaggccugguauauuagggauaacaggguaauaagaagacuucugcggucaacunnnnnn

SEQ ID NO: 149 - RNA, 665 nt, Artificial Sequence
  Corresponding RNA transcript 6
  variation join(2..6,660..665): /note=“Wherein n is A, C, G or U”
gnnnnncagcguucgcuacagcguucgcuauaacuauaacgguccuaagguagcgaaaugcaagagcaauaccgcccggaagagauagaauccaaaguacagcuucauagggauaacaggguaauuugggaugagaagcgcacauuugaaguaaccgaagacgagagcaaagagauaacuauaacgguccuaagguagcgaaaguauuacugccugucuaugcuucccuauccuucuggucgacuacacauguagggauaacaggguaauggccacguacguaacuacaccaucggugacgugaucgcccgcuaccagcguaacuauaacgguccuaagguagcgaauaugcugggcaaaaacguccugcagccgaucggcugggacgcguuuggucuagggauaacaggguaauugccugcggaaggcgcggcggugaaaaacaacaccgcuccggcaccgugguaacuauaacgguccuaagguagcgaaacguacgacaacaucgcguauaugaaaaaccagcucaaaaugcugggcuuuagggauaacaggguaauugguuaugacuggagccgcgagcuggcaaccuguacgccggaauacuaccuaacuauaacgguccuaagguagcgaaguugggaacagaaauucuucacnnnnnn

SEQ ID NO: 150 - RNA, 588 nt, Artificial Sequence
  Corresponding RNA transcript 7
  variation join(2..6,583..588): /note=“Wherein n is A, C, G or U”
gnnnnncagcguucgcuacagcguucgcuauaacuauaacgguccuaagguagcgaaaugcaagagcaauaccgcccggaagagauagaauccaaaguacagcuucauagggauaacaggguaauuugggaugagaagcgcacauuugaaguaaccgaagacgagagcaaagagauaacuauaacgguccuaagguagcgaaaguauuacugccugucuaugcuucccuauccuucuggucgacuacacauguagggauaacaggguaauggccacguacguaacuacaccaucggugacgugaucgcccgcuaccagcguaacuauaacgguccuaagguagcgaauaugcugggcaaaaacguccugcagccgaucggcugggacgcguuuggucuagggauaacaggguaauugccugcggaaggcgcggcggugaaaaacaacaccgcuccggcaccgugguaacuauaacgguccuaagguagcgaaacguacgacaacaucgcguauaugaaaaaccagcucaaaaugcugggcuuuagggauaacaggguaauugguuaugacuggagccgcgagnnnnnn

SEQ ID NO: 151 - RNA, 520 nt, Artificial Sequence
  Corresponding RNA transcript 8
  variation join(2..6,515..520): /note=“Wherein n is A, C, G or U”
gnnnnncagcguucgcuacagcguucgcuauaacuauaacgguccuaagguagcgaaaugcaagagcaauaccgcccggaagagauagaauccaaaguacagcuucauagggauaacaggguaauuugggaugagaagcgcacauuugaaguaaccgaagacgagagcaaagagauaacuauaacgguccuaagguagcgaaaguauuacugccugucuaugcuucccuauccuucuggucgacuacacauguagggauaacaggguaauggccacguacguaacuacaccaucggugacgugaucgcccgcuaccagcguaacuauaacgguccuaagguagcgaauaugcugggcaaaaacguccugcagccgaucggcugggacgcguuuggucuagggauaacaggguaauugccugcggaaggcgcggcggugaaaaacaacaccgcuccggcaccgugguaacuauaacgguccuaagguagcgaaacguacgacaacaucgcguauannnnnn

SEQ ID NO: 152 - RNA, 443 nt, Artificial Sequence
  Corresponding RNA transcript 9
  variation join(2..6,438..443): /note=“Wherein n is A, C, G or U”
gnnnnncagcguucgcuacagcguucgcuauaacuauaacgguccuaagguagcgaaaugcaagagcaauaccgcccggaagagauagaauccaaaguacagcuucauagggauaacaggguaauuugggaugagaagcgcacauuugaaguaaccgaagacgagagcaaagagauaacuauaacgguccuaagguagcgaaaguauuacugccugucuaugcuucccuauccuucuggucgacuacacauguagggauaacaggguaauggccacguacguaacuacaccaucggugacgugaucgcccgcuaccagcguaacuauaacgguccuaagguagcgaauaugcugggcaaaaacguccugcagccgaucggcugggacgcguuuggucuagggauaacaggguaauugccugcggaaggcgcggcggunnnnnn

SEQ ID NO: 153 - RNA, 375 nt, Artificial Sequence
  Corresponding RNA transcript 10
  variation join(2..6,370..375): /note=“Wherein n is A, C, G or U”
gnnnnncagcguucgcuacagcguucgcuauaacuauaacgguccuaagguagcgaaaugcaagagcaauaccgcccggaagagauagaauccaaaguacagcuucauagggauaacaggguaauuugggaugagaagcgcacauuugaaguaaccgaagacgagagcaaagagauaacuauaacgguccuaagguagcgaaaguauuacugccugucuaugcuucccuauccuucuggucgacuacacauguagggauaacaggguaauggccacguacguaacuacaccaucggugacgugaucgcccgcuaccagcguaacuauaacgguccuaagguagcgaauaugcugggcaaaaacguccugnnnnnn

SEQ ID NO: 154 - DNA, 35 bp, Artificial Sequence
  Forward PCR primer for all carrier molecules
  variation 19..23: /note=“Wherein n is A, C, G or T”
taatacgactcactatagnnnnncagcgttcgcta

SEQ ID NO: 155 - DNA, 18 bp, Artificial Sequence
  I-SceI recognition sequence 2
tagggataacagggtaat

SEQ ID NO: 156 - DNA, 26 bp, Artificial Sequence
  PCR_N6_r1
  variation 1..6: /note=“Wherein n is A, C, G or T”
nnnnnnctacgtgtcgcagacgaatt

SEQ ID NO: 157 - DNA, 26 bp, Artificial Sequence
  PCR_N6_r2
  variation 1..6: /note=“Wherein n is A, C, G or T”
nnnnnntatccagatcgttgagctgc

SEQ ID NO: 158 - DNA, 26 bp, Artificial Sequence
  PCR_N6_r3
  variation 1..6: /note=“Wherein n is A, C, G or T”
nnnnnncactgcgggatctctttacg

SEQ ID NO: 159 - DNA, 26 bp, Artificial Sequence
  PCR_N6_r4
  variation 1..6: /note=“Wherein n is A, C, G or T”
nnnnnngccgtcgataacttgttcgt

SEQ ID NO: 160 - DNA, 26 bp, Artificial Sequence
  PCR_N6_r5
  variation 1..6: /note=“Wherein n is A, C, G or T”
nnnnnnagttgaccgcagaagtcttc

SEQ ID NO: 161 - DNA, 26 bp, Artificial Sequence
  PCR_N6_r6
  variation 1..6: /note=“Wherein n is A, C, G or T”
nnnnnngtgaagaatttctgttccca

SEQ ID NO: 162 - DNA, 26 bp, Artificial Sequence
  PCR_N6_r7
  variation 1..6: /note=“Wherein n is A, C, G or T”
nnnnnnctcgcggctccagtcataac

SEQ ID NO: 163 - DNA, 26 bp, Artificial Sequence
  PCR_N6_r8
  variation 1..6: /note=“Wherein n is A, C, G or T”
nnnnnnctcgcggctccagtcataac

SEQ ID NO: 164 - DNA, 26 bp, Artificial Sequence
  PCR_N6_r9
  variation 1..6: /note=“Wherein n is A, C, G or T”
nnnnnnacchcchchccttcchcagg

SEQ ID NO: 165 - DNA, 26 bp, Artificial Sequence
  PCR_N6_r10
  variation 1..6: /note=“Wherein n is A, C, G or T”
nnnnnncaggacgtttttgcccagca

SEQ ID NO: 166 - DNA, 20 bp, Artificial Sequence
  Carrier f1 primer
gcggcagcgttcgctataac

SEQ ID NO: 167 - DNA, 20 bp, Artificial Sequence
  5′ adapter f1 primer
aatgatacggcgaccaccga

SEQ ID NO: 168 - DNA, 21 bp, Artificial Sequence
  3′ adapter r1 primer
caagcagaagacggcatacga

(19) United States
(12) Patent Application Publication
(10) Pub. No.: US 2021/0164020 A1
(43) Pub. Date: Jun. 3, 2021

(54) DEGRADABLE CARRIER NUCLEIC ACID FOR USE IN THE EXTRACTION, PRECIPITATION AND/OR PURIFICATION OF NUCLEIC ACIDS

(71) Applicant: Imperial College of Science, Technology and Medicine, London (GB)

(72) Inventors: Nevena Cvetesic, London (GB); Boris Lenhard, Richmond (GB)

(21) Appl. No.: 17/045,066

(22) PCT Filed: Apr. 1, 2019
(86) PCT No.: PCT/GB2019/050943

§ 371 Date: Oct. 2, 2020

(30) Foreign Application Priority Data
Apr. 5, 2018 (GB) 1805676.2

(51) Int. Cl. C12Q 1/6806; C12N 15/10

(52) U.S. Cl. CPC C12Q 1/6806; C12N 15/1065; C12N 15/1003

(57) ABSTRACT

The present invention provides a degradable carrier nucleic acid for use in the extraction, precipitation and/or purification of nucleic acids.

### FIELD OF INVENTION

The present invention is in the field of nucleic extraction and/or precipitation.

### INTRODUCTION

The extraction and/or precipitation and/or purification of nucleic acids, including single and double stranded RNA, DNA, siRNA and the like is hampered by the inevitable loss of sample (or target) nucleic acid during the extraction and/or precipitation and/or purification procedure. In fact, any step involving manipulation of the nucleic acid is considered to result in the loss of at least some sample nucleic acid. Whilst for some processes the loss of the sample nucleic acid can be tolerated, for example when isolating plasmid DNA from E. coli, in other situations, for example where the initial sample, for example sample of cells, is small, for example comprises a low number of cells or a low amount of initial nucleic acid, the loss of the sample can have significant effects on the data obtained, and can mean that some methods simply cannot be used for a particular sample since the resulting yield of nucleic acid will be too low.

One approach that may be used to increase the yield of a nucleic acid sample is through the use of a carrier. Such carriers include glycogen, nucleic acids and other inert non-interfering molecules which act to minimize sample loss caused by nonspecific adsorption and to improve specificity in affinity purification steps.

However, there are particular applications where the use of carrier nucleic acid is not favourable, for example where the resultant target nucleic acid is to be sequenced. Although sequencing of a target nucleic acid is possible in the presence of a carrier nucleic acid, due to the much higher abundance of the carrier nucleic acid to the target nucleic acid, up to 99% of the sequencing reads may come from the carrier rather than the target nucleic acid, which is wasteful and can make some experiments in some situations impossible to perform.

Another situation in which the use of currently available carrier nucleic acid is not desirable is in the production of cDNA libraries, for example in a method of Cap Analysis of Gene Expression (CAGE). In this case, much of the library inserts will be derived from the carrier nucleic acid, rather than the target nucleic acid, if the target sample is of low abundance (nanogram levels) compared to the carrier (microgram levels).

Cap analysis of gene expression (CAGE) is used for genome-wide quantitative identification of polymerase II transcription start sites (TSSs) at a single nucleotide resolution¹ as well as 5′end-centred expression profiling of RNA polymerase II (RNAPII) transcripts. The region surrounding a TSS (approximately 40 nucleotides upstream and downstream) represents the core promoter, where the transcription initiation machinery and general transcription factors bind to direct initiation by RNAPII². Information on exact TSS positions in the genome improves identification of core promoter sequences and led to the discovery of new core promoter and enhancer sequences³⁻⁵ (reviewed in Lenhard et al⁶ and Haberle et al⁷). The current knowledge of core promoter sequences identified by CAGE has uncovered their regulatory role on an unprecedented scale. CAGE detected TSS profiles represent an accurate and quantitative readout of promoter utilisation, their patterns reflect ontogenic, cell type specific and cellular homeostasis-associated dynamic profiles which allows promoter classification and inform about the diversity of promoter level regulation. This has led to increased use of CAGE techniques and their application in high profile research projects like ENCODE⁸, modENCODE⁹, FANTOM³, and FANTOM5¹⁰.

Central to CAGE methodology is the positive selection of RNA polymerase II transcripts using the cap-trapper technology¹¹. Briefly, this technology uses sodium periodate to selectively oxidize vicinal ribose diols present in the cap structure of mature mRNA transcripts, facilitating their subsequent biotinylation. RNA is first reversely transcribed using a random primer (N6TCT) and converted to RNA:cDNA hybrids, followed by oxidation, biotinylation and treatment with RNase I and RNase H to select only full-length RNA:cDNA hybrids; i.e. cDNA that has reached the 5′end of capped mRNA during reverse transcription will protect RNA against digestion. Purification of biotinylated RNA:cDNA hybrids is then performed using streptavidin-coated paramagnetic beads. These steps ensure that incompletely synthesized cDNA and cDNA synthesized from uncapped RNAs are eliminated from the sample. The initial CAGE protocol required large amounts of starting material (30-50 μg of total cellular RNA) and used restriction enzyme digestion to generate short reads (20 nucleotides)¹², whereas the later versions reduced the starting amount 10-fold and generated slightly longer reads (27 nucleotides) with increased mappability¹³.

The latest CAGE protocol using cap-trapping is nAnT-iCAGE¹⁴ and it is the most unbiased method for genome-wide identification of TSSs. It excludes PCR amplification as well as restriction enzymes used to produce short reads in previous CAGE versions. However, at least 5 μg of total RNA material is still required for nAnT-iCAGE. To address this, an alternative, biochemically unrelated approach, nanoCAGE, was developed for samples of limited material availability (50-500 ng of total cellular RNA)^{15,16}. NanoCAGE uses template switching¹⁷ instead of the cap-trapper technology to lower the starting material. Template switching is based on reverse transcriptase's ability to add extra cytosines complementary to the cap, which are then used for hybridization of the riboguanosine-tailed template switching oligonucleotide to extend and barcode only the 5′ full length cDNAs. Despite its simplicity, nanoCAGE has limitations that make it inferior to classic CAGE protocols: 1) template switching has been shown to be sequence dependent and therefore biased¹⁸, potentially compromising the determination of preferred TSS positions; 2) production of libraries from 50 ng of total RNA often requires 20-35 PCR amplification cycles, leading to low-complexity libraries with high levels of duplicates. Although nanoCAGE methodology implements unique molecular identifiers (UMIs)^{16, 19}, their use for removal of PCR duplicates is often complicated due to problems with achieving truly randomly synthesised UMI's and errors in sequencing²⁰.

Despite improvements in the CAGE methodology, the amount of input RNA needed for unbiased genome-wide identification of TSSs constitutes a true limitation when cells, and therefore RNA, are difficult to obtain. This is the case when working with embryonic tissue or early embryonic stages, rare cells types, FACS sorted selected cells, heterogeneous tumours, or diagnostic biopsies.

Accordingly there is a need for a suitable agent that can be used to increase the yield of target nucleic acid from a given sample, particularly in instances wherein the sample is a small sample that comprises little target nucleic acid, and so which allow methods such as CAGE to be performed on a greater variety of samples, including small samples.

The invention provides such an agent and methods of using the agent, as discussed below.

### BRIEF SUMMARY OF THE INVENTION

The inventors have surprisingly found that the use of carrier nucleic acid molecules that comprise at least one restriction site that is at least 15 nucleotides in length, is suitable for use in extracting, precipitating and/or purifying target nucleic acids. The carrier nucleic acid of the invention is designed so as to be degradable, yet is designed in such a way that the target nucleic acid is not degraded at all, or is degraded to only a minimal insignificant extent. The development of the nucleic acid of the invention has also allowed the inventors to develop a new method of CAGE, SLIC-CAGE or Super-Low Input Carrier-CAGE that allows for unbiased genome-wide promoterome analysis and generates complex, high quality libraries requiring 1000-fold less material than existing CAGE.

### DETAILED DESCRIPTION OF THE INVENTION

The present invention is as set out in the claims.

In a first aspect the invention provides a nucleic acid comprising at least a first sequence and a second sequence wherein at least one of the first or second sequence is an endonuclease recognition sequence or is capable of acting as an endonuclease recognition sequence when converted into a corresponding double-stranded DNA form, wherein the length of the endonuclease recognition sequence is at least 15 nucleotides.

The nucleic acid may be any kind of nucleic acid. For example it may be a single stranded DNA molecule, a single stranded RNA molecule, a double stranded DNA molecule or a double stranded RNA molecule, for instance.

In one embodiment the nucleic acid of the invention is an isolated nucleic acid.

In one embodiment the nucleic acid is a non-naturally occurring nucleic acid. By non-naturally occurring we include the meaning that the sequence of the nucleic acid of the invention is not found in nature. It will be appreciated that parts of the nucleic acid may be the same as naturally occurring nucleic acids, but the entire sequence of the nucleic acid of the invention is not found in nature, for example is not found in the genome of, or encoded by the genome of, any eukaryotic and/or prokaryotic organism. In some embodiments part of the sequence of the nucleic acid may be found in nature but the presence of the endonuclease recognition sequence as described herein means that the nucleic acid sequence in its entirety is not found in nature, for example is not found in the genome of any eukaryotic and/or prokaryotic organism. Indeed, the entire sequence of the nucleic acid of the invention may comprise naturally occurring sequences, but wherein the sequences are arranged in such a way so as the entire sequence is not found in nature, i.e. is not found as a continuous sequence in nature. The skilled person will understand that, through the use of the many available databases and search tools (for example the BLAST search tool at the National Center for Biotechnology Information website) it is possible to determine whether a particular nucleic acid sequence is found in the genome of a particular organism. The nucleic acids of the present invention are considered to be useful if they show less than about 90% homology or sequence identity to a particular genome, for example less than about 85% homology or sequence identity to a particular genome less than about 80% homology or sequence identity to a particular genome less than about 75% homology or sequence identity to a particular genome less than about 70% homology or sequence identity to a particular genome, for example to the genome of the organism that the nucleic acid of the invention is to be, for example, extracted from, precipitated from, or purified from.

It will be understood that preferably the carrier nucleic acid shows a sufficiently low degree of homology to the genome of the genome of the organism that the nucleic acid of the invention is to be, for example, extracted from, precipitated from, or purified from so that digestion of the carrier nucleic acid results in a low frequency of digestion of the genomic nucleic acid, for example less than 10 cleavage events, or less than 9 cleavage events, or less than 8 cleavage events, or less than 7 cleavage events, or less than 6 cleavage events, or less than 5 cleavage events, or less than 4 cleavage events, or less than 3 cleavage events, or less than 2 cleavage events, or less than 1 cleavage event occurring in the genomic nucleic acid. Preferably digestion results in no cleavage of the sample nucleic acid, for example the sample genomic nucleic acid.

Accordingly in another embodiment the nucleic acids of the present invention are considered to be useful if:
they show less than about 90% homology or sequence identity to a particular genome, for example less than about 85% homology or sequence identity to a particular genome less than about 80% homology or sequence identity to a particular genome less than about 75% homology or sequence identity to a particular genome less than about 70% homology or sequence identity to a particular genome, for example to the genome of the organism that the nucleic acid of the invention is to be, for example, extracted from, precipitated from, or purified from
across 100% of the length of the carrier nucleic acid sequence, or for example less than 100% of the length of the carrier nucleic acid sequence, for example less than 95% of the length of the carrier nucleic acid sequence, for example less than 90% of the length of the carrier nucleic acid sequence, for example less than 85% of the length of the carrier nucleic acid sequence, for example less than 80% of the length of the carrier nucleic acid sequence, for example less than 75% of the length of the carrier nucleic acid sequence, for example less than 70% of the length of the carrier nucleic acid sequence, for example less than 75% of the length of the carrier nucleic acid sequence, for example less than 70% of the length of the carrier nucleic acid sequence, for example less than 65% of the length of the carrier nucleic acid sequence, for example less than 60% of the length of the carrier nucleic acid sequence, for example less than 55% of the length of the carrier nucleic acid sequence, for example less than 50% of the length of the carrier nucleic acid sequence, for example less than 45% of the length of the carrier nucleic acid sequence, for example less than 40% of the length of the carrier nucleic acid sequence, for example less than 35% of the length of the carrier nucleic acid sequence, for example less than 30% of the length of the carrier nucleic acid sequence, for example less than 25% of the length of the carrier nucleic acid sequence, for example less than 20% of the length of the carrier nucleic acid sequence, for example less than 15% of the length of the carrier nucleic acid sequence, for example less than 10% of the length of the carrier nucleic acid sequence, for example less than 5% of the length of the carrier nucleic acid sequence, for example less than 2% of the length of the carrier nucleic acid sequence.

In one embodiment the degree of sequence homology between the carrier nucleic acid and the sample nucleic acid, for example the sample genomic nucleic acid, is lower than the minimum homology criteria used for mapping the sequences to the genome that the nucleic acid of the invention is to be used with, to, for example extract, precipitate or purify.

The skilled person will understand that when assessing the degree of % identity, the percentage of the query sequence covered (query cover) is also important. For example, the sequence identity may be found to be 90%, across only 20% of the carrier nucleic acid sequence, meaning that only 20% of the sequence is 90% identical to some part of the known organism/gene. In this instance, the carrier sequence would not be mapped to the target organism's genome.

In other embodiments the overall sequence of the nucleic acid of the invention is irrelevant, provided that the endonuclease site of the nucleic acid does not occur in the genome of the organism that the nucleic acid is to be used with, for example to extract, purify or precipitate, or does not occur with a high frequency. For example, the nucleic acid of the invention may be considered useful if the sequence of the endonuclease site of the nucleic acid, or endonuclease sites, occurs less than 10 times in the genome of the organism that the nucleic acid is to be used with, for example to extract, purify or precipitate, for example less than 9 times, for example less than 8 times, for example less than 7 times, for example less than 6 times, for example less than 5 times, for example less than 4 times, for examples less than 3 times, for example less than 2 times, for example less than 1 time, for example no times. As stated above, the skilled person will easily be able to determine whether a particular sequence occurs in the particular genome.

In one embodiment the nucleic acid of the invention consists or comprises a sequence according to any of SEQ ID NO: 143-153, or comprises or consists of a sequence that has at least 70% sequence identity or homology to any of SEQ ID NO: 143-153, for example at least 75% sequence identity or homology to any of SEQ ID NO: 143-153, for example at least 80% sequence identity or homology to any of SEQ ID NO: 143-153, for example at least 85% sequence identity or homology to any of SEQ ID NO: 143-153, for example at least 90% sequence identity or homology to any of SEQ ID NO: 143-153, for example at least 92% sequence identity or homology to any of SEQ ID NO: 143-153, for example at least 94% sequence identity or homology to any of SEQ ID NO: 143-153, for example at least 96% sequence identity or homology to any of SEQ ID NO: 143-153, for example at least 98% sequence identity or homology to any of SEQ ID NO: 143-153, for example 100% sequence identity or homology to any of SEQ ID NO: 143-153.

The nucleic acid of the invention is considered to be a degradable carrier nucleic acid, i.e. suitable for use in the extraction, purification, or precipitation of nucleic acid from a sample, for example from a crude cell lysate or other such sample and wherein the carrier nucleic acid can be specifically degraded prior to downstream applications, such as sequencing or library formation, wherein the degradation results in carrier nucleic acid fragments that do not interfere with the downstream applications or which are removable through, for example size selection. When used in this way the type of nucleic acid molecule, for instance single or double stranded, RNA or DNA, is typically selected so as to be comparable to the nucleic acid molecule that is being extracted, purified or precipitated (the target nucleic acid). Accordingly, when extracting, purifying or precipitating for example:

mRNA from a sample of cells, the nucleic acid of the invention should be a single stranded RNA;
Single stranded DNA from a sample, the nucleic acid of the invention should be single stranded DNA;
Double stranded RNA, the nucleic acid of the invention should be double stranded RNA;
Double stranded DNA, the nucleic acid of the invention should be double stranded DNA;
Double stranded plasmid DNA, the nucleic acid of the invention should be double stranded circular DNA.

It is preferable if the nucleic acid also comprises other structures that allow the nucleic acid of the invention to more closely mimic the target nucleic acid, for example 5′ caps, which will be discussed further later.

The skilled person will appreciate that endonucleases typically recognise specific sequences in double stranded DNA molecules. Accordingly, where the nucleic acid of the invention is a single stranded nucleic acid, for example a single stranded DNA or RNA, it is not strictly correct to state that these molecules comprise recognition sites for one or more endonucleases. Accordingly, in these instances it is more accurate to state that those single stranded nucleic acids of the invention comprise sequences that are capable of acting as an endonuclease recognition sequence when converted into a corresponding double-stranded DNA form, through for example reverse transcription of RNA into cDNA and second strand synthesis to make the double-stranded form. Accordingly therefore the invention provides a single stranded nucleic acid, for example a single stranded DNA or RNA, that comprises at least one sequence that although not capable being recognised and cleaved by an endonuclease when in the single stranded form is able to be recognised and cleaved once the single stranded nucleic acid is converted into double stranded DNA. The skilled person will understand the interconversion between RNA and DNA and will understand the sequence requirements of the RNA molecule such that once converted to double stranded DNA the site can be recognised and cleaved.

The nucleic acid molecules of the invention are capable of being specifically degraded through the use of endonucleases. Whilst in some applications degradation of the carrier nucleic acid to any degree may be sufficient, in other applications it is preferred that the resultants products from the carrier nucleic acid degradation are removable. The skilled person will be aware of such techniques to remove nucleic acid fragments from a sample, for example through the use of gel electrophoresis or various commercial size exclusion kits. In a preferred embodiment the nucleic acid fragments are removed by Solid Phase Reversible Immobilization, for example Solid Phase Reversible Immobilization-based paramagnetic beads, for example AMPure beads or RNAclean XP beads.

Such removal of the carrier nucleic acid fragments following endonuclease digestion is considered to be more efficient if the fragments are 200 nucleotides in length or less. The skilled person will readily be able to determine the optimum length of nucleic acid, frequency and position of the endonuclease sites which result in cleaved fragments of 200 nucleotides or less. In preferred embodiments the nucleic acid length and frequency and position of endonuclease sites are arranged so as to allow for less than 100% cleavage efficiency of a particular enzyme and so are, for example, arranged so as to result in fragments that are (based on assumed 100% cleavage efficiency) all less than 200 nucleotides, for example less than 180 nucleotides, for example less than 160 nucleotides, for example less than 140 nucleotides, for example less than 120 nucleotides, for example less than 100 nucleotides, for example less than 80 nucleotides, for example less than 60 nucleotides, for example less than 40 nucleotides, for example less than 20 nucleotides. In this way the skilled person can ensure that the fragments that require removal from the sample are all below the threshold size of whichever method is being used to remove the fragments, for example are all below 200 nucleotides when the fragments are to be removed by Solid Phase Reversible Immobilization, for example Solid Phase Reversible Immobilization-based paramagnetic beads, for example AMPure beads or RNAclean XP beads.

Accordingly in one embodiment the nucleic acid of the invention is such that cleavage of the second sequence results in the production of nucleic acid fragments that are all less than 200 nucleotides, for example less than 180 nucleotides, for example less than 160 nucleotides, for example less than 140 nucleotides, for example less than 120 nucleotides, for example less than 100 nucleotides, for example less than 80 nucleotides, for example less than 60 nucleotides, for example less than 40 nucleotides, for example less than 20 nucleotides.

Accordingly in another embodiment the nucleic acid of the invention is such that cleavage of the second sequence results in the production of nucleic acid fragments that are all less than 200 nucleotides, for example less than 180 nucleotides, for example less than 160 nucleotides, for example less than 140 nucleotides, for example less than 120 nucleotides, for example less than 100 nucleotides, for example less than 80 nucleotides, for example less than 60 nucleotides, for example less than 40 nucleotides, for example less than 20 nucleotides.

In preferred embodiments the nucleic acid of the invention comprises at least two sequences that are endonuclease recognition sequences or that are capable of acting as an endonuclease recognition sequences when converted into a corresponding double-stranded DNA form. In this way it is possible to either arrange both sequences such that cleavage of both sequences at 100% efficiency results in fragments that are all less than 200 nucleotides, for example less than 180 nucleotides, for example less than 160 nucleotides, for example less than 140 nucleotides, for example less than 120 nucleotides, for example less than 100 nucleotides, for example less than 80 nucleotides, for example less than 60 nucleotides, for example less than 40 nucleotides, for example less than 20 nucleotides. However, it is also possible to use both sequences in such a way as to account for less than 100% cleavage but still result in fragments that are less than 200 nucleotides as discussed above.

Accordingly in one embodiment the at least two sequences that are endonuclease recognition sequences or that are capable of acting as an endonuclease recognition sequences when converted into a corresponding double-stranded DNA form do not overlap with one another i.e. each sequence is capable of being cleaved by an endonuclease even if the other sequence has already been cleaved. In other words, the endonuclease digestion of one recognition sequence does not affect the integrity of a neighbouring recognition sequence.

In one embodiment the at least two sequences that are endonuclease recognition sequences or that are capable of acting as an endonuclease recognition sequences when converted into a corresponding double-stranded DNA form are not part of a multiple cloning site (MCS). The skilled person will understand what is meant by the term MCS. MCS's are also called polylinkers and are short segments of DNA that comprise several, for example up to around 20 endonuclease sites which are typically restriction sites, as will be clear to the skilled person and typically do not comprise endonuclease sites that are at least 15 nucleotides in length. For example a MCS may comprise at least 4 endonuclease sites, for example at least 6 endonuclease sites, for example at least 8 endonuclease sites, for example at least 10 endonuclease sites, for example at least 12 endonuclease sites, for example at least 14 endonuclease sites, for example at least 16 endonuclease sites, for example at least 18 endonuclease sites, for example at least 20 endonuclease sites, for example at least 22 endonuclease sites, for example at least 24 endonuclease sites, for example at least 26 endonuclease sites, for example at least 28 endonuclease sites, for example at least 30 endonuclease sites, within a region of nucleic acid sequence of less than 500 nucleotides, for example less than 400 nucleotides, for example less than 300 nucleotides, for example less than 250 nucleotides, for example less than 200 nucleotides, for example less than 175 nucleotides, for example less than 150 nucleotides, for example less than 125 nucleotides, for example less than 100 nucleotides, for example less than 75 nucleotides, for example less than 50 nucleotides, for example less than 25 nucleotides.

In one embodiment, if the nucleic acid comprises a multiple cloning site the multiple cloning site does not comprise the first and/or second sequence. For example, if the nucleic acid comprises a region of nucleic acid sequence of less than 500 nucleotides, for example less than 400 nucleotides, for example less than 300 nucleotides, for example less than 250 nucleotides, for example less than 200 nucleotides, for example less than 175 nucleotides, for example less than 150 nucleotides, for example less than 125 nucleotides, for example less than 100 nucleotides, for example less than 75 nucleotides, for example less than 50 nucleotides, for example less than 25 nucleotides, that comprises at least 4 endonuclease sites, for example at least 6 endonuclease sites, for example at least 8 endonuclease sites, for example at least 10 endonuclease sites, for example at least 12 endonuclease sites, for example at least 14 endonuclease sites, for example at least 16 endonuclease sites, for example at least 18 endonuclease sites, for example at least 20 endonuclease sites, for example at least 22 endonuclease sites, for example at least 24 endonuclease sites, for example at least 26 endonuclease sites, for example at least 28 endonuclease sites, for example at least 30 endonuclease sites, the region of nucleic acid does not comprise the first and/or sequence, i.e. does not comprise the first and/or second sequences that are endonuclease recognition sequences or that are capable of acting as an endonuclease recognition sequences when converted into a corresponding double-stranded DNA form wherein the first and second sequences are at least 15 nucleotides in length.

In another embodiment the at least two sequences that are endonuclease recognition sequences or that are capable of acting as an endonuclease recognition sequences when converted into a corresponding double-stranded DNA form are the only endonuclease recognition sequences found in the nucleic acid.

Accordingly in another embodiment the nucleic acid of the invention is such that cleavage of the first sequence and the second sequence results in the production of nucleic acid fragments that are all less than 200 nucleotides, for example less than 180 nucleotides, for example less than 160 nucleotides, for example less than 140 nucleotides, for example less than 120 nucleotides, for example less than 100 nucleotides, for example less than 80 nucleotides, for example less than 60 nucleotides, for example less than 40 nucleotides, for example less than 20 nucleotides. This also applies to embodiments where the nucleic acid comprises more than two sequences that are endonuclease recognition sequences or that are capable of acting as an endonuclease recognition sequences when converted into a corresponding double-stranded DNA form endonuclease sites.

In a preferred embodiment, cleavage of the first and/or second sequence (and or one or more of any additional endonuclease recognition sequences) results in the production of nucleic acid fragments that are all less than 80 nucleotides, for example less than 70 nucleotides.

In one embodiment the first sequence is a sequence that is capable of acting as an endonuclease recognition sequence when converted into a corresponding double-stranded DNA form and the second sequence is not a sequence that is capable of acting as an endonuclease recognition sequence when converted into a corresponding double-stranded DNA form. In this instance the nucleic acid of the invention comprises only one endonuclease site. Such a nucleic acid molecule is considered to be useful as a degradable carrier nucleic acid. However, preferably the nucleic acid of the invention comprises more than one endonuclease recognition sequence or more than one sequence that is capable of acting as an endonuclease recognition sequence when converted into a corresponding double-stranded DNA form. In this embodiment a single carrier nucleic acid molecule can be cleaved twice, preferably cleaved from a long fragment into 2 or 3 shorter fragments that are easier to remove from the sample as discussed above.

The nucleic acid of the invention may have any number of endonuclease recognition sequences or sequences that are capable of acting as an endonuclease recognition sequence when converted into a corresponding double-stranded DNA form. Accordingly, in one embodiment the nucleic acid comprises at least three sequences wherein each sequence is an endonuclease recognition sequence or is capable of acting as an endonuclease recognition sequence when converted into a corresponding double-stranded DNA form, wherein the length of each endonuclease recognition sequence is at least 15 nucleotides, for example wherein the nucleic acid comprises at least 4, for example at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 350, at least 400, at least 450, at least 500, at least 550, at least 600, at least 650, or at least 700 sequences wherein each sequence is an endonuclease recognition sequence or is capable of acting as an endonuclease recognition sequence when converted into a corresponding double-stranded DNA form, and wherein the length of each endonuclease recognition sequence is at least 15 nucleotides. Each of these sequences may be the same, or may be different.

The length of the nucleic acid may be any length. The skilled person can determine the optimal length of nucleic acid or nucleic acids of the invention required. Typically the length of the nucleic acid is 10 kb or less, for example less than 9.5 kb, for example less than 9.0 kb, for example less than 8.5 kb, for example less than 8.0 kb, for example less than 7.5 kb, for example less than 7.0 kb, for example less than 6.5 kb, for example less than 6.0 kb, for example less than 5.5 kb, for example less than 5.0 kb, for example less than 4.5 kb, for example less than 4.0 kb, for example less than 3.5 kb, for example less than 3.0 kb, for example less than 2.5 kb, for example less than 2.0 kb, for example less than 1.5 kb, for example less than 1.25 kb, for example less than 1.0 kb, for example less than 900 bp, for example less than 800 bp, for example less than 700 bp, for example less than 600 bp, for example less than 500 bp, for example less than 400 bp, for example less than 300 bp, for example less than 200 bp, for example less than 100 bp.

For example, the length of the nucleic acid according to the invention may be between 100 bp and 10 kb in length, for example between 200 bp and 9 kb, for example between 300 bp and 8 kb, for example between 400 bp and 7 kb, for example between 500 bp and 6 kb, for example between 600 bp and 5 kb, for example between 700 bp and 4 kb, for example between 800 bp and 3 kb, for example between 900 bp and 2 kb, for example 1 kb.

Since degradation of the target sample, i.e. the nucleic acid that is being precipitated, extracted or purified for example is undesirable, it is preferred if the nucleic acid of the invention comprises endonuclease recognition sequences or sequences that are capable of acting as an endonuclease recognition sequence when converted into a corresponding double-stranded DNA form that are not found in the target sample or that are only found to a low, insignificant frequency. If the nucleic acid of the invention comprised recognition sequences for standard restriction enzymes, which typically have a recognition sequence length of 4-8 nucleotides the predicted frequency of cutting in, for example, the human genome would be: 802978.5 times. Clearly, the use of such an enzyme to digest the carrier nucleic acid is undesirable since the target nucleic acid would likely be significantly degraded. The inventors have determined that the minimum endonuclease recognition sequence length that can be used to largely or entirely avoid cleavage of the target nucleic acid is 15 nucleotides. For example an 18 base pair recognition sequence will occur only once in every 7×10¹⁰ base pairs of random sequence. This is equivalent to only one site in 20 mammalian-sized genomes.

Accordingly the nucleic acids of the invention comprise one or more endonuclease recognition sequences or sequences that are capable of acting as an endonuclease recognition sequence when converted into a corresponding double-stranded DNA wherein the recognition sequence is at least 15 nucleotides in length. The recognition sequence length can be any length, provided that it is at least 15 nucleotides in length.

In one embodiment the nucleic acid of the invention comprises at least a first and second sequence, wherein at least the first sequence is an endonuclease recognition sequence or that is capable of acting as an endonuclease recognition sequence when converted into a corresponding double-stranded DNA form, and wherein the first sequence that is an endonuclease recognition sequence or that is capable of acting as an endonuclease recognition sequence when converted into a corresponding double-stranded DNA form is at least 16 nucleotides or greater in length, for example at least 17 nucleotides or greater in length, for example at least 18 nucleotides or greater in length, for example at least 19 nucleotides or greater in length, for example at least 20 nucleotides or greater in length, for example at least 21 nucleotides or greater in length, for example at least 22 nucleotides or greater in length, for example at least 23 nucleotides or greater in length, for example at least 24 nucleotides or greater in length, for example at least 25 nucleotides or greater in length, for example at least 26 nucleotides or greater in length, for example at least 27 nucleotides or greater in length, for example at least 28 nucleotides or greater in length, for example at least 29 nucleotides or greater in length, for example at least 30 nucleotides or greater in length, for example at least 31 nucleotides or greater in length, for example at least 32 nucleotides or greater in length, for example at least 33 nucleotides or greater in length, for example at least 34 nucleotides or greater in length, for example at least 35 nucleotides or greater in length,

for example wherein both of the first sequence and a second sequence that are endonuclease recognition sequences or that are capable of acting as endonuclease recognition sequences when converted into a corresponding double-stranded DNA form are at least 16 nucleotides or greater in length, for example at least 17 nucleotides or greater in length, for example at least 18 nucleotides or greater in length, for example at least 19 nucleotides or greater in length, for example at least 20 nucleotides or greater in length, for example at least 21 nucleotides or greater in length, for example at least 22 nucleotides or greater in length, for example at least 23 nucleotides or greater in length, for example at least 24 nucleotides or greater in length, for example at least 25 nucleotides or greater in length, for example at least 26 nucleotides or greater in length, for example at least 27 nucleotides or greater in length, for example at least 28 nucleotides or greater in length, for example at least 29 nucleotides or greater in length, for example at least 30 nucleotides or greater in length, for example at least 31 nucleotides or greater in length, for example at least 32 nucleotides or greater in length, for example at least 33 nucleotides or greater in length, for example at least 34 nucleotides or greater in length, for example at least 35 nucleotides or greater in length,
for example wherein any number of or all of the sequences that are endonuclease recognition sequences or that are capable of acting as endonuclease recognition sequences when converted into a corresponding double-stranded DNA form are at least 16 nucleotides or greater in length, for example at least 17 nucleotides or greater in length, for example at least 18 nucleotides or greater in length, for example at least 19 nucleotides or greater in length, for example at least 20 nucleotides or greater in length, for example at least 21 nucleotides or greater in length, for example at least 22 nucleotides or greater in length, for example at least 23 nucleotides or greater in length, for example at least 24 nucleotides or greater in length, for example at least 25 nucleotides or greater in length, for example at least 26 nucleotides or greater in length, for example at least 27 nucleotides or greater in length, for example at least 28 nucleotides or greater in length, for example at least 29 nucleotides or greater in length, for example at least 30 nucleotides or greater in length, for example at least 31 nucleotides or greater in length, for example at least 32 nucleotides or greater in length, for example at least 33 nucleotides or greater in length, for example at least 34 nucleotides or greater in length, for example at least 35 nucleotides or greater in length.

In some embodiments, one or more of the sequences that are endonuclease recognition sequences or that are capable of acting as endonuclease recognition sequences when converted into a corresponding double-stranded DNA forms are the same length.

In one embodiment then the nucleic acid of the invention comprises at least a first and second sequence that are endonuclease recognition sequences or that are capable of acting as endonuclease recognition sequences when converted into a corresponding double-stranded DNA form are the same length, for example wherein any number of or all of the sequences that are endonuclease recognition sequences or that are capable of acting as endonuclease recognition sequences when converted into a corresponding double-stranded DNA form are the same length.

In other embodiments, two or more of the sequences that are endonuclease recognition sequences or that are capable of acting as endonuclease recognition sequences when converted into a corresponding double-stranded DNA form are of different lengths, for example any number of or all of the sequences that are endonuclease recognition sequences or that are capable of acting as endonuclease recognition sequences when converted into a corresponding double-stranded DNA form are of different lengths.

In some embodiments the nucleic acid of the invention comprises sequences that are endonuclease recognition sequences or that are capable of acting as endonuclease recognition sequences when converted into a corresponding double-stranded DNA form of at least three different lengths, for example at least four different lengths, for example at least five different lengths, for example at least six different lengths, for example at least seven different lengths, for example at least eight different lengths, for example at least nine different lengths, for example at least 10 different lengths.

In some preferred embodiments, the nucleic acid of the invention comprises at least two sequences that are endonuclease recognition sequences or that are capable of acting as endonuclease recognition sequences when converted into a corresponding double-stranded DNA form wherein the sequences are identical. In other embodiments any number of or all of the sequences that are endonuclease recognition sequences or that are capable of acting as endonuclease recognition sequences when converted into a corresponding double-stranded DNA form are identical to one another.

In some embodiments, the nucleic acid of the invention comprises at least two sequences that are endonuclease recognition sequences or that are capable of acting as endonuclease recognition sequences when converted into a corresponding double-stranded DNA form that are different to one another, for example wherein any number of or all of the sequences that are endonuclease recognition sequences or that are capable of acting as endonuclease recognition sequences when converted into a corresponding double-stranded DNA form are different to one another.

As will be apparent to the skilled person, any of the embodiments described herein may be combined. Accordingly in one embodiment the nucleic acid of the invention comprises at least two sequences that are endonuclease recognition sequences or that are capable of acting as endonuclease recognition sequences when converted into a corresponding double-stranded DNA form that are different to one another, and at least two sequences that are endonuclease recognition sequences or that are capable of acting as endonuclease recognition sequences when converted into a corresponding double-stranded DNA form that are identical to one another.

As will be apparent, in one embodiment the nucleic acid of the invention comprises at least two sequences that are endonuclease recognition sequences or that are capable of acting as endonuclease recognition sequences when converted into a corresponding double-stranded DNA form are recognition sequences for the same endonuclease, for example any number of or all of the sequences that are endonuclease recognition sequences or that are capable of acting as endonuclease recognition sequences when converted into a corresponding double-stranded DNA form are recognition sequences for the same endonucleases.

In another or the same embodiment the nucleic acid of the invention comprises at least two sequences that are endonuclease recognition sequences or that are capable of acting as endonuclease recognition sequences when converted into a corresponding double-stranded DNA form are recognition sequences for different endonucleases, for example any number of or all of the sequences that are endonuclease recognition sequences or that are capable of acting as endonuclease recognition sequences when converted into a corresponding double-stranded DNA form are recognition sequences for different endonucleases.

It is considered preferable that the nucleic acid of the invention comprises sequences for at least two different endonucleases at least because it is considered to increase the efficiency of carrier degradation as discussed above, and also to reduce the repetitive nature of the carrier sequence. High repetitiveness complicates the carrier gene synthesis process and may reduce efficiency of PCR used to prepare templates for in vitro transcription, and also efficiency of in vitro transcription due to higher probability of secondary structure formation.

In one preferred embodiment the number of endonuclease recognition sequences (preferably relating to more than one endonuclease) spanning the carrier sequence is chosen to produce 77 nucleotides fragments (with accounted 100% cutting efficiency).

Fragments of that size can be easily removed through size-selection using for example AMPure beads. A 1 kb nucleic acid therefore requires 14 endonuclease recognitions sites. As discussed above, to reduce repetitiveness imposed by the recognitions site, it is preferred to combine 2 endonuclease restriction sites as they differ in sequence.

It will be clear then that the same endonuclease recognition sequence or sequence that is capable of acting as endonuclease recognition sequences when converted into a corresponding double-stranded DNA form can be repeated within the nucleic acid, for example the first and/or the second sequence occurs at least twice within the nucleic acid, for example at least three times, for example at least four times, for example at least five times, for example at least 6 times, for example at least 7 times, for example at least 8 times, for example at least 9 times, for example at least 10 times, for example at least 12 times, for example at least 14 times, for example at least 16 times, for example at least 18 times, for example at least 20 times, for example at least 25 times, for example at least 30 times, for example at least 35 times, for example at least 40 times, for example at least 45 times, for example at least 50 times, for example at least 55 times, for example at least 60 times, for example at least 65 times, for example at least 70 times, for example at least 75 times, for example at least 80 times, for example at least 85 times, for example at least 90 times, for example at least 95 times, for example at least 100 times, for example at least 110 times, for example at least 120 times, for example at least 130 times, for example at least 140 times, for example at least 150 times, for example at least 160 times, for example at least 170 times, for example at least 180 times, for example at least 190 times, for example at least 200 times.

In a further embodiment, the nucleic acid of the invention comprises sequences that are endonuclease recognition sequences or that are capable of acting as endonuclease recognition sequences when converted into a corresponding double-stranded DNA form are recognition sequences for at least three different endonucleases, for example at least four different endonucleases, for example at least five different endonucleases, for example at least six different endonucleases, for example at least seven different endonucleases, for example at least eight different endonucleases, for example at least nine different endonucleases, for example at least 10 different endonucleases.

It will be apparent that the nucleic acid of the invention can comprise at least two sequences that are endonuclease recognition sequences or that are capable of acting as endonuclease recognition sequences when converted into a corresponding double-stranded DNA form that are identical to one another, and can also comprise at least two sequences that are endonuclease recognition sequences or that are capable of acting as endonuclease recognition sequences when converted into a corresponding double-stranded DNA form that are different to one another. These sequences may be arranged in any manner. For example, the sequences may be repeated in the nucleic acid in alternating fashion, for example wherein where the nucleic acid comprises at least three sequences that are endonuclease recognition sequences or are capable of acting as endonuclease recognition sequences when converted into a corresponding double-stranded DNA form each of the sequences are repeated in an alternating fashion. Any arrangement of the sequences is contemplated within the present invention, and the most suitable arrangement for the particular application can be easily determined by the skilled person.

Any endonuclease enzyme and corresponding recognition sequence is considered to be useful in the present invention, provided that the recognition sequence is at least 15 nucleotides in length. Accordingly, the skilled person will be able to identify suitable enzymes, for example which include any suitable restriction enzymes and also the homing endonucleases, which are known to have longer recognition sequences than the restriction enzymes. Homing endonuclease recognition sites are extremely rare. For example, an 18 base pair recognition sequence will occur only once in every 7×10¹⁰ base pairs of random sequence. This is equivalent to only one site in 20 mammalian-sized genomes.

In one embodiment the only endonuclease recognition sequences or the sequences that are capable of acting as endonuclease recognition sequences when converted into a corresponding double-stranded DNA form found in the nucleic acid are at least 15 nucleotides in length.

Accordingly, in one embodiment the nucleic acid of the invention comprises at least one sequence that is an endonuclease recognition sequence or that is capable of acting as an endonuclease recognition sequence when converted into a corresponding double-stranded DNA form wherein the sequence is a recognition sequence for a homing endonuclease, for example may comprise two sequences that are endonuclease recognition sequences or that are capable of acting as endonuclease recognition sequences when converted into a corresponding double-stranded DNA form wherein the sequences are recognition sequences for homing endonuclease enzymes, for example wherein all of the sequences that are endonuclease recognition sequences or that are capable of acting as endonuclease recognition sequences when converted into a corresponding double-stranded DNA form are recognition sequences for homing endonuclease enzymes. The skilled person will understand what is meant by homing endonuclease enzymes, and some suitable examples are:

BneMS4ORFIP, F-CphI, F-EcoT3I, F-EcoT5I, F-EcoT5II, F-EcoT5IV, F-PhiU5I, F-SceI, F-SceII, F-TevI, F-TevII, F-TevIII, F-TevIV, H-DreI, H-DreI, I-AabMI, I-AchMI, I-AniI, I-ApeKI, I-BanI, I-BasI, I-BmoI, I-Bth0305I, I-BthII, 1-BthORFAP, I-CeuI, I-ChuI, I-CmoeI, I-CpaI, I-CpaII, I-CpaMI, I-CreI, I-CreII, I-CsmI, I-CvuI, I-DdiI, I-DmoI, I-GpeMI, I-GpiI, I-GzeI, I-GzeII, I-HjeMI, I-HmuI, I-HmuII, 1-LlaI, I-LtrI, I-LtrWI, I-MpeMI, I-MsoI, I-NanI, I-NfiI, I-NitI, I-NjaI, I-OmiII, I-OnuI, I-PakI, I-PanMI, I-PfoP3I, I-PnoMI, I-PogTE7I, I-PorI, I-PpoI, I-ScaI, I-SceI, I-SceII, I-SceIII, I-SceIV, I-SceV, I-SceVI, I-SceVII, I-SecIII, I-SmaMI, I-SpomI, I-SscMI, I-Ssp6803I, I-TevI, I-TevII. I-TevIII. I-TsII. I-TsIWI, I-Tsp061I, I-TwoI, I-Vdi141I, -AvaI, PI-BciPI, PI-HvoWI, PI-MtuI, PI-PabI, PI-PabII, PI-PfuI, PI-PfuII, PI-PkoI, PI-PkoII, PI-PspI, PI-PspI, PI-ScaI, PI-SceI, PI-TfuI, PI-TfuII, PI-ThyI, PI-TliI, PI-TliII, PI-TmaI, PI-TmaKI, PI-ZbaI.

Accordingly any one, two, more or all of the sequences that are endonuclease recognition sequences or that are capable of acting as endonuclease recognition sequences when converted into a corresponding double-stranded DNA form are homing endonuclease recognition sequences, for example are selected from the group consisting of BneMS4ORFIP, F-CphI, F-EcoT3I, F-EcoT5I, F-EcoT5II, F-EcoT5IV, F-PhiU5I, F-SceI, F-SceII, F-TevI, F-TevII, F-TevIII, F-TevIV, H-DreI, H-DreI, I-AabMI, I-AchMI, I-AniI, I-ApeKI, I-BanI, I-BasI, I-BmoI, I-Bth0305I, I-BthII, 1-BthORFAP, I-CeuI, I-ChuI, I-CmoeI, I-CpaI, I-CpaII, I-CpaMI, I-CreI, I-CreII, I-CsmI, I-CvuI, I-DdiI, I-DmoI, I-GpeMI, I-GpiI, I-GzeI, I-GzeII, I-HjeMI, I-HmuI, I-HmuII, 1-LlaI, I-LtrI, I-LtrWI, I-MpeMI, I-MsoI, I-NanI, I-NfiI, I-NitI, I-NjaI, I-OmiII, I-OnuI, I-PakI, I-PanMI, I-PfoP3I, I-PnoMI, I-PogTE7I, I-PorI, I-PpoI, I-ScaI, I-SceI, I-SceII, I-SceIII, I-SceIV, I-SceV, I-SceVI, I-SceVII, I-SecIII, I-SmaMI, I-SpomI, I-SscMI, I-Ssp6803I, I-TevI, I-TevII. I-TevIII. I-TsII. I-TsIWI, I-Tsp061I, I-TwoI, I-Vdi141I, -AvaI, PI-BciPI, PI-HvoWI, PI-MtuI, PI-PabI, PI-PabII, PI-PfuI, PI-PfuII, PI-PkoI, PI-PkoII, PI-PspI, PI-PspI, PI-ScaI, PI-SceI, PI-TfuI, PI-TfuII, PI-ThyI, PI-TliI, PI-TliII, PI-TmaI, PI-TmaKI, PI-ZbaI.

The recognition sequences for the above enzymes are described by SEQ ID Nos: 1-142, see for example FIG. 28. Accordingly, in one embodiment, the first and/or second, any of or all of the sequences that are endonuclease recognition sequences or that are capable of acting as endonuclease recognition sequences when converted into a corresponding double-stranded DNA form are recognition sequences is selected from the group consisting of: SEQ ID NO: 1-142, for example SEQ ID NO: 1 and/or SEQ ID NO: 2.

It will be appreciated that the nucleic acid of the invention may comprise one or more modifications. This may be to allow the nucleic acid to more closely mimic the target nucleic acid. For example, in some applications the target nucleic acid will have been modified, for example may have been biotinylated. In this case it is preferable if the nucleic acid of the invention comprises a similar modification. Accordingly, in one embodiment the nucleic acid of the invention is modified, for example is biotinylated.

As discussed above, the nucleic acid of the invention is suitable for use in the extraction, purification and/or precipitation of mRNA molecules, for example as a step in a method of rtPCR, CAGE or other application which requires isolation of the mRNA molecules. In these instances where for example the nucleic acid is an RNA the RNA may comprise a 5′ cap. The term 5′ cap is well known in the art. In other embodiments where the nucleic acid is an RNA and does not comprise a 5′ cap. Preferably, as discussed further below, in such instances a composition will be used that comprises both capped and uncapped nucleic acids of the invention, since this is considered to more closely mimic the target nucleic acid population.

The present invention also provides a vector comprising the nucleic acid according to the invention, or comprises a sequence that is capable of being transcribed into an RNA transcript, wherein the transcript comprises a nucleic acid according to the invention. For instance, where the nucleic acid of the invention is a double stranded DNA, the vector may directly comprise this nucleic acid, for instance as part of a plasmid sequence. Where the nucleic acid of the invention is an RNA, the RNA of the invention may be directly incorporated into a vector, such as an RNA vector, or, the DNA sequence that when transcribed results in the production of the RNA may be incorporated into a DNA vector, such as a plasmid.

Preferences for the nucleic acid are as discussed above.

The invention also provides a cell comprising a nucleic acid according to the invention or a vector according to the invention, for example wherein the cell is:

a) a prokaryotic cell, for example a bacterial cell, for example an E. coli cell, a Bacillus subtilis cell, a Bacillus megaterium cell, a Vibrio natriegens cell, or a Pseudomonas fluorescens cell; or
b) a eukaryotic cell, for example
a yeast cell, for example Pichia pastoris or Saccharomyces cerevisiae;
an insect cell, for example a baculovirus infected insect cell; or
a mammalian cell, for example a baculovirus infected mammalian cell, a HEK293 cell, a HeLa cell, or CHO cells.

Preferences for the nucleic acid and vector are as discussed above.

Other suitable cells include any cell capable of expressing the RNA of the invention or propagating the DNA of the invention. In vitro transcription systems may also be used, along with purely synthetic means of producing nucleic acids.

The culture of such cells and subsequent isolation of nucleic acid is at least one way in which the nucleic acids of the invention can be produced. It will become apparent that the degradable nucleic acid carrier of the present invention may be used to isolate and prepare further nucleic acids of the invention. In this case the endonuclease sites that are present in both the carrier nucleic acid and the target carrier nucleic acid that is to be extracted, purified or precipitated must be directed towards different endonucleases.

As discussed above, the invention also provides a composition comprising at least one nucleic acid according to the invention. Preferences for the nucleic acid are as discussed above.

In one embodiment the composition comprises at least two nucleic acids according to the invention, wherein the at least two nucleic acids are of different sequence to one another, for example the composition comprises at least 3 different nucleic acids wherein the at least 3 different nucleic acids are of different sequence to one another, for example comprising at least 4 different nucleic acids wherein the at least 4 different nucleic acids are of different sequence to one another, for example comprising at least 5 different nucleic acids wherein the at least 5 different nucleic acids are of different sequence to one another, for example comprising at least 6 different nucleic acids wherein the at least 6 different nucleic acids are of different sequence to one another, for example comprising at least 7 different nucleic acids wherein the at least 7 different nucleic acids are of different sequence to one another, for example comprising at least 8 different nucleic acids wherein the at least 8 different nucleic acids are of different sequence to one another, for example comprising at least 9 different nucleic acids wherein the at least 9 different nucleic acids are of different sequence to one another, for example comprising at least 10 different nucleic acids wherein the at least 10 different nucleic acids are of different sequence to one another.

The invention also provides a composition comprising at least two nucleic acids according to the invention wherein the at least 2 nucleic acids are of different length, for example at least 3 nucleic acids according to the invention wherein the at least 3 nucleic acids are of different lengths, for example at least 4 nucleic acids according to the invention wherein the at least 4 nucleic acids are of different lengths, for example at least 5 nucleic acids according to the invention wherein the at least 5 nucleic acids are of different lengths, for example at least 6 nucleic acids according to the invention wherein the at least 6 nucleic acids are of different lengths, for example at least 7 nucleic acids according to the invention wherein the at least 7 nucleic acids are of different lengths, for example at least 8 nucleic acids according to the invention wherein the at least 8 nucleic acids are of different lengths, for example at least 9 nucleic acids according to the invention wherein the at least 9 nucleic acids are of different lengths, for example at least 10 nucleic acids according to the invention wherein the at least 10 nucleic acids are of different lengths.

In one embodiment the composition comprises 10 different nucleic acids according to the invention wherein each of the 10 nucleic acids is of a different length, for example wherein the composition comprises a nucleic acid according to the invention of each of the following lengths:

i) between 1000 nucleotides and 1200 nucleotides, for example 1100 nucleotides, for example 1034 nucleotides;
ii) between 900 nucleotides and 1000 nucleotides, for example between 920 nucleotides and 980 nucleotides, for example between 960 nucleotides and 970 nucleotides, for example 966 nucleotides;
iii) between 850 nucleotides and 900 nucleotides, for example between 860 nucleotides and 890 nucleotides, for example 889 nucleotides;
iv) between 800 nucleotides and 850 nucleotides, for example between 810 nucleotides and 840 nucleotides, for example between 820 nucleotides and 830 nucleotides, for example 821 nucleotides;
v) between 700 nucleotides and 800 nucleotides, for example between 720 nucleotides and 780 nucleotides, for example between 740 nucleotides and 760 nucleotides, for example 744 nucleotides;
vi) between 650 nucleotides and 700 nucleotides, for example between 660 nucleotides and 690 nucleotides, for example between 670 nucleotides and 680 nucleotides, for example 676 nucleotides;
vii) between 550 nucleotides and 650 nucleotides, for example between 560 nucleotides and 640 nucleotides, for example between 570 nucleotides and 630 nucleotides, for example between 580 nucleotides and 620 nucleotides, for example between 590 nucleotides and 610 nucleotides, for example 599 nucleotides or 600 nucleotides;
viii) between 500 nucleotides and 550 nucleotides, for example between 510 nucleotides and 540 nucleotides, for example between 520 nucleotides and 530 nucleotides, for example 531 nucleotides;
ix) between 400 nucleotides and 500 nucleotides, for example between 410 nucleotides and 490 nucleotides, for example between 420 nucleotides and 480 nucleotides, for example between 430 nucleotides and 470 nucleotides, for example between 440 nucleotides and 460 nucleotides, for example 450 nucleotides or 454 nucleotides; and
x) between 300 nucleotides and 400 nucleotides, for example between 310 nucleotides and 390 nucleotides, for example between 320 nucleotides and 380 nucleotides, for example between 330 nucleotides and 370 nucleotides, for example between 340 nucleotides and 360 nucleotides, for example 350 nucleotides or 386 nucleotides;
for example wherein the composition comprises at least 10 different nucleic acids according to the invention wherein the nucleic acids are 1034 nucleotides in length, 966 nucleotides in length, 889 nucleotides in length, 821 nucleotides in length, 744 nucleotides in length, 676 nucleotides in length, 599 nucleotides in length, 531 nucleotides in length, 454 nucleotides in length, and 386 nucleotides in length.

As discussed above, it is preferable that in certain applications the features of the carrier nucleic acid mimic, at least to some extent, the features of the target nucleic acid population. This is at least since if, for example, a target population comprises both biotinylated and non-biotinylated RNA, a bias in the amount of biotinylated versus non-biotinylated RNA that is lost during the extraction procedure, for example, for example through preferential adherence to plasticware, may occur. In addition, if targeted purification of biotinylated molecules is performed (as in CAGE using Streptavidin coated magnetic beads), the carrier would be lost in that purification step and would not act as the carrier throughout the protocol if it was not biotinylated and purified along with the sample molecules. If the carrier nucleic acid only comprised biotinylated RNA, then the resultant target nucleic acid preparation may be skewed. The same applies the percentage of capped versus uncapped RNAs. In one embodiment the composition comprises capped RNA nucleic acids according to the invention. In one embodiment the composition comprises uncapped RNA nucleic acids according to the invention. However in another embodiment the composition comprises both capped RNA nucleic acids according to the invention and uncapped RNA nucleic acids according to the invention.

Accordingly, one embodiment provides a composition wherein the percentage of RNA nucleic acids that comprise a 5′ cap is similar to the percentage of capped RNA nucleic acids in a sample. The skilled person will be aware of suitable methods to arrive at the desired percentage capping, some of which are detailed in the Examples.

For example, the composition may be such that at least 5% of the RNA nucleic acids comprises a 5′ cap, for example at least 10% of the RNA nucleic acids comprises a 5′ cap, for example at least 20% of the RNA nucleic acids comprises a 5′ cap, for example at least 30% of the RNA nucleic acids comprises a 5′ cap, for example at least 40% of the RNA nucleic acids comprises a 5′ cap, for example at least 50% of the RNA nucleic acids comprises a 5′ cap, for example at least 60% of the RNA nucleic acids comprises a 5′ cap, for example at least 70% of the RNA nucleic acids comprises a 5′ cap, for example at least 80% of the RNA nucleic acids comprises a 5′ cap, for example at least 90% of the RNA nucleic acids comprises a 5′ cap, for example 100% of the RNA nucleic acids comprises a 5′ cap.

In one embodiment the composition of the invention comprises a range of different sized nucleic acids wherein the range of sizes of the nucleic acids according to the invention are similar to the range of sizes of RNA or DNA nucleic acids in a sample. This is again considered to reduce bias in loss of particular target nucleic acids.

The skilled person will appreciate that the present invention is ideally suited to being provided as a kit. Preferences discussed above apply equally to this aspect.

For example, in one embodiment the kit comprises at least one nucleic acid according to the invention, for example comprising at least two nucleic acids according to the invention, for example comprising at least 3 nucleic acids according to the invention, for example comprising at least 4 nucleic acids according to the invention, for example comprising at least 5 nucleic acids according to the invention, for example comprising at least 6 nucleic acids according to the invention, for example comprising at least 7 nucleic acids according to the invention, for example comprising at least 8 nucleic acids according to the invention, for example comprising at least 9 nucleic acids according to the invention, for example comprising at least 10 nucleic acids according to the invention; and/or

at least one vector according to the invention; and/or
at least one cell according to the invention; and/or
at least one composition according to the invention.

In one embodiment the kit comprises at least one nucleic acid according to the invention that is a capped RNA and at least one nucleic acid according to the invention that is an uncapped RNA. The kit may comprise 2 different RNA nucleic acids according to the invention, each in both a capped and uncapped form,
for example wherein the kit comprises 3 different RNA nucleic acids according to the invention, each in both a capped and uncapped form,
for example wherein the kit comprises 4 different RNA nucleic acids according to the invention, each in both a capped and uncapped form,
for example wherein the kit comprises 5 different RNA nucleic acids according to the invention, each in both a capped and uncapped form,
for example wherein the kit comprises 6 different RNA nucleic acids according to the invention, each in both a capped and uncapped form,
for example wherein the kit comprises 7 different RNA nucleic acids according to the invention, each in both a capped and uncapped form,
for example wherein the kit comprises 8 different RNA nucleic acids according to the invention, each in both a capped and uncapped form,
for example wherein the kit comprises 9 different RNA nucleic acids according to the invention, each in both a capped and uncapped form,
for example wherein the kit comprises 10 different RNA nucleic acids according to the invention, each in both a capped and uncapped form.

The kit may comprise a at least two nucleic acids according to the invention wherein the at least two nucleic acids are of different lengths, for example at least 3 nucleic acids according to the invention wherein the at least 3 nucleic acids are of different lengths, for example at least 4 nucleic acids according to the invention wherein the at least 4 nucleic acids are of different lengths, for example at least 5 nucleic acids according to the invention wherein the at least 5 nucleic acids are of different lengths, for example at least 6 nucleic acids according to the invention wherein the at least 6 nucleic acids are of different lengths, for example at least 7 nucleic acids according to the invention wherein the at least 7 nucleic acids are of different lengths, for example at least 8 nucleic acids according the invention wherein the at least 8 nucleic acids are of different lengths, for example at least 9 nucleic acids according to the invention wherein the at least 9 nucleic acids are of different lengths, for example at least 10 nucleic acids according to the invention wherein the at least 10 nucleic acids are of different lengths.

The kit according to the invention may also comprise one or more endonuclease enzymes, for example wherein at least one of the endonuclease enzymes is a homing endonuclease enzyme, preferably wherein the at least one endonuclease enzyme recognises at least one of the sequences that are endonuclease recognition sequences or that are capable of acting as endonuclease recognition sequences when converted into a corresponding double-stranded DNA form.

The skilled person will understand that the kit may also comprise any one or more agents that are useful in the applications which the nucleic acid of the invention will be used in, for examples agents that are required for CAGE, ChIP or library preparation. For example, the kit may comprise any one or more of:
SPRI-paramagnetic beads (solid-phase reversible immobilization)
dNTP mix
Reverse Transcriptase
RNase H
Exonuclease I
USER Enzyme
A polymerase, for example Taq polymerase, DeepVent (exo-) DNA polymerase and/or Phusion High Fidelity DNA polymerase
A phosphatase, for example Shrimp alkaline phosphatase (SAP)
a ribonuclease, for example RNaseONE™ Ribonuclease
a DNA ligase
paramagnetic beads, for example Streptavidin paramagnetic beads
Dimethyl sulfoxide (DMSO)
2-Propanol
EDTA (Ethylenediaminetetraacetic acid)
NaCl (sodium chloride)
Polyoxyethylene(20) Sorbitan Monolaurate (Tween20).
NaOH (sodium hydroxide)
Tris (Trizma base)
NaCAc (sodium acetate)
Glycerol
tRNA (transfer RNA)
DNasel (Deoxiribonuclease, RNase free)
Ethanol
Proteinase
Sorbitol
Trehalose dehydrate
NalO4 (sodium periodate)
Biotin hydrazide
wash buffers for streptavidin paramagnetic beads
reverse transcription primer
5′ linker
3′linker
second strand synthesis primer
reagents to determine the quantity of single stranded DNA/cDNA, for example Qμant-iT™ Oligreen [symbol] ssDNA Reagent and Kit
reagents to determine the quantity of double stranded DNA, for example Qμant-iT™ PicoGreen [symbol] dsDNA Reagent and Kit
agents for the isolation of genomic DNA or RNA, for example TRIzol, glass beads, RNeasy kit (Qiagen)
a forward and/or reverse primers designed to amplify the carrier nucleic acid from a nucleic acid according to any of claims 1-28 or a vector according to any of claims 29 and 30, for example amplification by PCR DNA gel extraction and PCR purification kit (example Qiagen)
agents for in vitro transcription, for example a T7 based promoter RNA synthesis kit, for example HiScribe T7 High Yield RNA Synthesis Kit SP6, T7 and T3
Agents for in vitro RNA capping, for example Vaccinia Capping System from NEB
Agents for purification of RNA, for example an RNeasy kit

As will be apparent, the nucleic acids of the present invention can be advantageously used in a number of different methods. The preferences discussed above apply equally to any of the methods described herein.

Accordingly the invention provides a method of isolating nucleic acid from a sample wherein the method comprises the use of any one or more of: the nucleic acids according to the invention; the vectors according to the invention; the cell according to the invention; the compositions according the invention; and/or the kits according to the invention. The method may further comprise contacting the nucleic acid or the composition with at least one endonuclease, for example at least one homing endonuclease.

The invention also provides a method for improving the yield of nucleic acid obtained from a sample, wherein the method comprises the use of any one or more of: the nucleic acids according to the invention; the vectors according to the invention; the cell according to the invention; the compositions according the invention; and/or the kits according to the invention. The sample may be considered to be a small sample, for example a sample that comprises:

0.1 ng to 500 ng of nucleic acids; and/or
less than 5000 cells, for example less than 4000 cells, for example less than 2000 cells, for example less than 1000 cells, for example less than 800 cells, for example less than 600 cells, for example less than 400 cells, for example less than 200 cells, for example around 100 cells or less;
a sample from an embryo; a sample of oocytes, FACS sorted cells, rare cell types, small biopsies, primordial germ cells, and samples of an embryo in the early embryonic developmental stages.

The method may further comprise contacting the nucleic acid or the composition with at least one endonuclease, for example at least one homing endonuclease.

The invention also provides a method for isolating nucleic acid that will be sequenced wherein the method comprises the use of any one or more of: the nucleic acids according to the invention; the vectors according to the invention; the cell according to the invention; the compositions according the invention; and/or the kits according to the invention. The method may further comprise contacting the nucleic acid or the composition with at least one endonuclease, for example at least one homing endonuclease.

The invention also provides a method for sequencing a nucleic acid wherein the method comprises the use of any one or more of: the nucleic acids according to the invention; the vectors according to the invention; the cell according to the invention; the compositions according the invention; and/or the kits according to the invention. The method may further comprise contacting the nucleic acid or the composition with at least one endonuclease, for example at least one homing endonuclease.

The invention also provides a method for cap analysis of gene expression (CAGE) wherein the method comprises the use of any one or more of: the nucleic acids according to the invention; the vectors according to the invention; the cell according to the invention; the compositions according the invention; and/or the kits according to the invention. The method may further comprise contacting the nucleic acid or the composition with at least one endonuclease, for example at least one homing endonuclease. In one embodiment said contacting occurs following reverse transcription of the RNA to DNA.

The invention also provides a new method for cap analysis of gene expression (CAGE). The new method for CAGE is intended to improve sequencing efficiency and shorten the protocol. Fewer protocol steps should lead to higher complexity libraries achieved with lower amount of total cellular RNA (currently the protocol is optimised to work with 5-10 ng, and optimisations may allow use of 1-2 ng). The average fragment length in the final SLIC-CAGE libraries is typically around 800 nucleotides. Clustering of fragments on Illumina sequencers is more efficient for shorter fragments (standard Illumina sequencing libraries tend to have fragment size 200-500 nucleotides), therefore larger fragments typically lower sequencing efficiency. To improve sequencing quality of the SLIC-CAGE libraries, a tagmentation step (Illumina Nextera XT kit) is incorporated. Tagmentation relies on transposition of barcode sequences randomly into DNA in a “cut and paste” reaction. The use of the Illumina system is not considered to be essential and any equivalent tagmentation step is considered to be appropriate. This random insertion efficiently fragments the DNA and at the same time adds the sequences required for PCR amplification and sequencing. We have tested incorporation of the tagmentation step after the SLIC-CAGE protocol is performed. Analysis of the resultant libraries is underway.

Accordingly, in one embodiment the method comprises cleavage of the cDNA by a transposon and tagging of the double-stranded cDNA, for example wherein the method comprises tagmentation. In a preferred embodiment the method comprises the use of any one or more of:

the nucleic acids according to the invention; the vectors according to the invention; the cell according to the invention; the compositions according the invention; and/or the kits according to the invention. The method may further comprise contacting the nucleic acid or the composition with at least one endonuclease, for example at least one homing endonuclease. In one embodiment said contacting occurs following reverse transcription of the RNA to DNA.

In one embodiment of this method, unlike some known methods of CAGE (nAnT-iCAGE protocol), the method does not comprise a 3′ linker ligation reaction and/or does not comprise uracil specific excision reagent (USER) treatment. The skilled person will understand that USER is used to cut the nucleic acid at uracil positions and is typically used in the CAGE protocol to remove the upper strand of the long 3′ adapter region—thereby it is cut in smaller pieces and easier to remove by heat denaturation.

Accordingly, in one embodiment, the method of CAGE comprises the following steps in the following order:

### A)

1. cleavage of the cDNA by a transposon and tagging of the double-stranded cDNA, for example tagmentation;
2. PCR amplification
3. Degradation of the carrier nucleic acid according to the invention or the nucleic acid of the compositions according to the invention;
4. Purification of the DNA fragments, for example by Solid Phase Reversible Immobilization-based paramagnetic beads, for example AMPure beads or RNAclean XP; or

### B)

1. Degradation of the nucleic acid according to the invention or the nucleic acid of the compositions according to the invention;
2. Purification of the DNA fragments, for example by Solid Phase Reversible Immobilization-based paramagnetic beads, for example AMPure beads or RNAclean XP; or
3. cleavage of the cDNA by a transposon and tagging of the double-stranded cDNA, for example tagmentation;
4. PCR amplification; or

### C)

1. Degradation of the carrier nucleic acid according to the invention or the nucleic acid of the compositions according to the invention;
2. Purification of the DNA fragments, for example by Solid Phase Reversible Immobilization-based paramagnetic beads, for example AMPure beads or RNAclean XP;
3. PCR amplification
4. (optional 2nd round of degradation of the carrier nucleic acid according to the invention or the nucleic acid of the compositions according to the invention)
5. Cleavage of the cDNA by a transposon and tagging of the double-stranded cDNA, for example tagmentation.

In method C the original CAGE/SLIC-CAGE method is not shortened, but the tagmentation step is added to optimise sequencing.

The invention also provides a method for assessing gene promoters and/or transcription start sites, the method comprising:
a) providing a sample of target nucleic acid; and
b) mixing the sample of nucleic acid with one or more nucleic acids according to the invention, or a composition according to the invention.

The invention also provides a method of generating a nucleic acid library, for example a cDNA library, wherein the method comprises the use of any one or more of: the nucleic acids according to the invention; the vectors according to the invention; the cell according to the invention; the compositions according the invention; and/or the kits according to the invention. The method may further comprise contacting the nucleic acid or the composition with at least one endonuclease, for example at least one homing endonuclease.

The invention also provides a method of diagnosis wherein the method comprises the use of any one or more of: the nucleic acids according to the invention; the vectors according to the invention; the cell according to the invention; the compositions according the invention; and/or the kits according to the invention. The method may further comprise contacting the nucleic acid or the composition with at least one endonuclease, for example at least one homing endonuclease.

The invention also provides a method of chromatin immunoprecipitation (ChIP), ChIP-seq, or FARP-ChIP-seq wherein the method comprises the use of any one or more of: the nucleic acids according to the invention; the vectors according to the invention; the cell according to the invention; the compositions according the invention; and/or the kits according to the invention. The method may further comprise contacting the nucleic acid or the composition with at least one endonuclease, for example at least one homing endonuclease.

The sample used in any of the above methods may be considered to be a small sample, for example a sample that comprises:

0.1 ng to 500 ng of nucleic acids; and/or
less than 5000 cells, for example less than 4000 cells, for example less than 2000 cells, for example less than 1000 cells, for example less than 800 cells, for example less than 600 cells, for example less than 400 cells, for example less than 200 cells, for example around 100 cells or less;
a sample from an embryo; a sample of oocytes, FACS sorted cells, rare cell types, small biopsies, primordial germ cells, and samples of an embryo in the early embryonic developmental stages.

The listing or discussion of an apparently prior-published document in this specification should not necessarily be taken as an acknowledgement that the document is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention. For example, the nucleic acid of the invention may comprise any number of sequences that are endonuclease recognition sequences or that are capable of acting as an endonuclease recognition sequences when converted into a corresponding double-stranded DNA form, and so the nucleic acid of the invention may comprise, for example 50 recognition sequences wherein 10 of them have a length of 20 nucleotides, 5 have a length of 23 nucleotides, and 25 have a length of 30 nucleotides.

The nucleic acid of the invention may also be a single stranded RNA molecule comprising two alternating sequences that are capable of acting as an endonuclease recognition sequences when converted into a corresponding double-stranded DNA form, wherein each sequence is a recognition site for a different homing endonuclease, and wherein the RNA comprises a 5′ cap and is biotinylated.

### FIGURE LEGENDS

FIG. 1. SLIC-CAGE development and assessment. (a) Schematics of the SLIC-CAGE approach. Target RNA of limited quantity is mixed with the carrier mix to get 5 μg of total RNA material. cDNA is synthesised through reverse transcription and the cap oxidized using sodium periodate. Oxidation allows attachment of biotin using biotin hydrazide. In addition to the cap structure, biotin gets attached to the mRNA's 3′ end, as it is also oxidized using sodium periodate. To remove biotin from mRNA:cDNA hybrids with incompletely synthesized cDNA, and from mRNA's 3′ ends, the samples are treated with RNase I and RNase H. Complete cDNAs (cDNA that reached the 5′ end of mRNA), are selected by affinity purification on streptavidin magnetic beads (cap-trapping). cDNA is released from cap-trapped cDNA:mRNA hybrids and 5′- and 3′-linkers are ligated. The library molecules that originate from the carrier are degraded using I-Sce-I and I-Ceu-I homing endonucleases and the fragments removed using AMPure beads. The leftover library molecules are then PCR amplified to increase the amount of material for sequencing. (b-c) Pearson correlation of nAnT-iCAGE and SLIC-CAGE libraries prepared from (b) 5 ng or (c) 10 ng of S. cerevisiae total RNA. (d) Pearson correlation of SLIC-CAGE technical replicates prepared from 10 ng of S. cerevisiae total RNA. (e) CTSS signal in example locus on chromosome 12 in SLIC-CAGE libraries prepared from 5 or 10 ng of S. cerevisiae total RNA, and in nAnT-iCAGE library prepared from standard 5 □g of total RNA. The inset grey boxes show a magnification of a tag cluster. (f-h) Pearson correlation of nAnT-iCAGE and SLIC-CAGE libraries prepared from (f) 5 ng, (g) 10 ng or (h) 25 ng of M. musculus total RNA from. (i) CTSS signal in example locus on chromosome 8 in SLIC-CAGE libraries prepared from 5, 10 or 25 ng of M. musculus total RNA, and in the reference nAnT-iCAGE library prepared from standard 5 μg of total RNA. The inset grey boxes show a magnification of a tag cluster.

FIG. 2. Identifying the lower limits of SLIC-CAGE libraries. (a) Genomic locations of tag clusters identified in SLIC-CAGE libraries prepared from 1, 5 or 10 ng of S. cerevisiae total RNA versus the reference nAnT-iCAGE library. (b) Distribution of tag cluster interquantile widths in SLIC-CAGE libraries prepared from 1, 5 or 10 ng of S. cerevisiae total RNA and in the nAnT-iCAGE library. (c) Nucleotide composition of all CTSSs identified in SLIC-CAGE libraries prepared from 5 or 10 ng of S. cerevisiae total RNA and in the reference nAnT-iCAGE library. (d) Dinucleotide composition of all CTSSs (left panel) or dominant CTSSs (right panel) identified in SLIC-CAGE libraries prepared from 5 or 10 ng of S. cerevisiae total RNA and in the nAnT-iCAGE library. Both panels are ordered from the most to the least used dinucleotide in nAnT-iCAGE. (e) Genomic locations of tag clusters in SLIC-CAGE libraries prepared from 5, 10 or 25 ng of M. musculus total RNA and in the nAnT-iCAGE library. (f) Distribution of tag cluster interquantile widths in SLIC-CAGE libraries prepared from 5, 10 or 25 ng of M. musculus total RNA and the nAnT-iCAGE library. (g) Nucleotide composition of all CTSSs identified in SLIC-CAGE libraries prepared from 5, 10 or 25 ng of M. musculus total RNA or identified in the nAnT-iCAGE library. (h) Dinucleotide composition of all CTSSs (left panel) or dominant CTSSs (right panel) identified in SLIC-CAGE libraries prepared from 5, 10 or 25 ng of M. musculus total RNA or identified in the reference nAnT-iCAGE library. Both panels are ordered from the most to the least used dinucleotide in the reference nAnT-iCAGE.

FIG. 3. Comparison of nanoCAGE and the reference nAnT-iCAGE. (a-e) Pearson correlation of nAnT-iCAGE and nanoCAGE libraries prepared from (a) 5 ng, (b, c) 10 ng, (d) 50 ng or (e) 500 ng of S. cerevisiae total RNA. (f) Pearson correlation of nanoCAGE technical replicates prepared from 10 ng of S. cerevisiae total RNA. (g) CTSS signal in example locus on chromosome 12 in nanoCAGE libraries prepared from 5, 10, 50 or 500 ng, SLIC-CAGE library prepared from 5 ng, and the nAnT-iCAGE library prepared from 5 μg of S. cerevisiae total RNA (the same locus is shown in FIG. 1e). Insets in first two nanoCAGE libraries have a different scale, as signal is skewed with PCR amplification. The inset grey boxes show a magnification of a tag cluster. Different tag cluster is magnified compared to FIG. 1e, as nanoCAGE did not detect the upstream tag cluster on the minus strand (h) Genomic locations of tag clusters identified in nanoCAGE libraries prepared from 5-500 ng of S. cerevisiae total RNA and in the nAnT-iCAGE library (i) Distribution of tag cluster interquantile widths in nanoCAGE libraries prepared from 5-500 ng of S. cerevisiae total RNA versus the reference nAnT-iCAGE library. (j) Nucleotide composition of all CTSSs identified in nanoCAGE libraries prepared from 5-500 ng of S. cerevisiae total RNA or identified in the reference nAnT-iCAGE library. (k) Dinucleotide composition of all CTSSs (left panel) or dominant CTSSs (right panel) identified in nanoCAGE libraries prepared from 5-500 ng of S. cerevisiae total RNA or in the reference nAnT-iCAGE library. Both panels are ordered from the most to the least used dinucleotide in the reference nAnT-iCAGE.

FIG. 4. SLIC-CAGE is equivalent to nAnT-iCAGE for pattern discovery. Comparison of SLIC-CAGE derived from 10 ng and nAnT-iCAGE derived from 5 μg of M. musculus total RNA. In all heatmaps, promoters are centred at the dominant CTSS (dashed vertical line at 0) and ordered by tag cluster interquantile width with sharpest promoters on top and broadest on the bottom of each heatmap. The horizontal line separates sharp and broad promoters (empirical boundary for sharp promoters is set at interquantile width <=3). (a) Comparison of TA dinucleotide density in the SLIC-CAGE (left) and nAnT-iCAGE library (right). (b) Comparison of TATA-box density in SLIC-CAGE (left) vs nAnT-iCAGE library (right). Promoter regions are scanned using a minimum of 80th percentile match to the TATA-box pwm. (c) Comparison of GC dinucleotide density in the SLIC-CAGE (left) and nAnT-iCAGE library (right). (d) Average VWV (AA/AT/TA/TT) dinucleotide frequency in sharp and broad promoters identified in SLIC-CAGE (left) or nAnT-iCAGE library (right). Inset shows a closer view on VVVV dinucleotide frequency (blue) overlain with the signal obtained when the sequences are aligned to a randomly chosen identified CTSS within broad promoters (yellow). (e) CTSS coverage heatmap of SLIC-CAGE (left) or nAnT-iCAGE library (right). (f) H3K4me3 relative coverage in sharp versus broad promoters identified in SLIC-CAGE (left) or nAnT-iCAGE (right). (g) H3K4me3 signal density across promoter regions centred on SLIC-CAGE or nAnT-iCAGE identified dominant CTSS. (h) Relative coverage of CpG islands across sharp and broad promoters, centred on dominant CTSS identified in SLIC-CAGE (left) or nAnT-iCAGE (right). (i) CpG islands coverage signal across promoter regions centred on dominant CTSS identified in SLIC-CAGE (left) or nAnT-iCAGE (right).

FIG. 5

Sequence of the carrier synthetic gene. I-SceI recognition sites are underlined, while I-CeuI recognitions sites are highlighted in bold.

[SEQ ID NO: 143]
CAGCGTTCGCTATAACTATAACGGTCCTAAGGTAGCGAAATGCAAGAGCA
ATACCGCCCGGAAGAGATAGAATCCAAAGTACAGCTTCATAGGGATAACA
GGGTAATTTGGGATGAGAAGCGCACATTTGAAGTAACCGAAGACGAGAGC
AAAGAGATAACTATAACGGTCCTAAGGTAGCGAAAGTATTACTGCCTGTC
TATGCTTCCCTATCCTTCTGGTCGACTACACATGTAGGGATAACAGGGTA
ATGGCCACGTACGTAACTACACCATCGGTGACGTGATCGCCCGCTACCAG
CGTAACTATAACGGTCCTAAGGTAGCGAATATGCTGGGCAAAAACGTCCT
GCAGCCGATCGGCTGGGACGCGTTTGGTCTAGGGATAACAGGGTAATTGC
CTGCGGAAGGCGCGGCGGTGAAAAACAACACCGCTCCGGCACCGTGGTAA
CTATAACGGTCCTAAGGTAGCGAAACGTACGACAACATCGCGTATATGAA
AAACCAGCTCAAAATGCTGGGCTTTAGGGATAACAGGGTAATTGGTTATG
ACTGGAGCCGCGAGCTGGCAACCTGTACGCCGGAATACTACCTAACTATA
ACGGTCCTAAGGTAGCGAAGTTGGGAACAGAAATTCTTCACCGAGCTGTA
TAAAAAAGGCCTGGTATATTAGGGATAACAGGGTAATAAGAAGACTTCTG
CGGTCAACTGGTGCCCGAACGACCAGACCGTACTGGCTAACTATAACGGT
CCTAAGGTAGCGAAGAACGAACAAGTTATCGACGGCTGCTGCTGGCGCTG
CGATACCAAAGTTGTAGGGATAACAGGGTAATAACGTAAAGAGATCCCGC
AGTGGTTTATCAAAATCACTGCTTACGCTGACTAACTATAACGGTCCTAA
GGTAGCGAATTGCAGCTCAACGATCTGGATAAACTGGATCACTGGCCAGA
CACCGTTAATAGGGATAACAGGGTAATCGAATTCGTCTGCGACACGTAG
Sequence of corresponding RNA transcript [SEQ ID
NO: 144]:
RNA_1:
GNNNNNCAGCGUUCGCUACAGCGUUCGCUAUAACUAUAACGGUCCUAAGG
UAGCGAAAUGCAAGAGCAAUACCGCCCGGAAGAGAUAGAAUCCAAAGUAC
AGCUUCAUAGGGAUAACAGGGUAAUUUGGGAUGAGAAGCGCACAUUUGAA
GUAACCGAAGACGAGAGCAAAGAGAUAACUAUAACGGUCCUAAGGUAGCG
AAAGUAUUACUGCCUGUCUAUGCUUCCCUAUCCUUCUGGUCGACUACACA
UGUAGGGAUAACAGGGUAAUGGCCACGUACGUAACUACACCAUCGGUGAC
GUGAUCGCCCGCUACCAGCGUAACUAUAACGGUCCUAAGGUAGCGAAUAU
GCUGGGCAAAAACGUCCUGCAGCCGAUCGGCUGGGACGCGUUUGGUCUAG
GGAUAACAGGGUAAUUGCCUGCGGAAGGCGCGGCGGUGAAAAACAACACC
GCUCCGGCACCGUGGUAACUAUAACGGUCCUAAGGUAGCGAAACGUACGA
CAACAUCGCGUAUAUGAAAAACCAGCUCAAAAUGCUGGGCUUUAGGGAUA
ACAGGGUAAUUGGUUAUGACUGGAGCCGCGAGCUGGCAACCUGUACGCCG
GAAUACUACCUAACUAUAACGGUCCUAAGGUAGCGAAGUUGGGAACAGAA
AUUCUUCACCGAGCUGUAUAAAAAAGGCCUGGUAUAUUAGGGAUAACAGG
GUAAUAAGAAGACUUCUGCGGUCAACUGGUGCCCGAACGACCAGACCGUA
CUGGCUAACUAUAACGGUCCUAAGGUAGCGAAGAACGAACAAGUUAUCGA
CGGCUGCUGCUGGCGCUGCGAUACCAAAGUUGUAGGGAUAACAGGGUAAU
AACGUAAAGAGAUCCCGCAGUGGUUUAUCAAAAUCACUGCUUACGCUGAC
UAACUAUAACGGUCCUAAGGUAGCGAAUUGCAGCUCAACGAUCUGGAUAA
ACUGGAUCACUGGCCAGACACCGUUAAUAGGGAUAACAGGGUAAUCGAAU
UCGUCUGCGACACGUAGNNNNNN
RNA_2:
[SEQ ID NO: 145]
GNNNNNCAGCGUUCGCUACAGCGUUCGCUAUAACUAUAACGGUCCUAAGG
UAGCGAAAUGCAAGAGCAAUACCGCCCGGAAGAGAUAGAAUCCAAAGUAC
AGCUUCAUAGGGAUAACAGGGUAAUUUGGGAUGAGAAGCGCACAUUUGAA
GUAACCGAAGACGAGAGCAAAGAGAUAACUAUAACGGUCCUAAGGUAGCG
AAAGUAUUACUGCCUGUCUAUGCUUCCCUAUCCUUCUGGUCGACUACACA
UGUAGGGAUAACAGGGUAAUGGCCACGUACGUAACUACACCAUCGGUGAC
GUGAUCGCCCGCUACCAGCGUAACUAUAACGGUCCUAAGGUAGCGAAUAU
GCUGGGCAAAAACGUCCUGCAGCCGAUCGGCUGGGACGCGUUUGGUCUAG
GGAUAACAGGGUAAUUGCCUGCGGAAGGCGCGGCGGUGAAAAACAACACC
GCUCCGGCACCGUGGUAACUAUAACGGUCCUAAGGUAGCGAAACGUACGA
CAACAUCGCGUAUAUGAAAAACCAGCUCAAAAUGCUGGGCUUUAGGGAUA
ACAGGGUAAUUGGUUAUGACUGGAGCCGCGAGCUGGCAACCUGUACGCCG
GAAUACUACCUAACUAUAACGGUCCUAAGGUAGCGAAGUUGGGAACAGAA
AUUCUUCACCGAGCUGUAUAAAAAAGGCCUGGUAUAUUAGGGAUAACAGG
GUAAUAAGAAGACUUCUGCGGUCAACUGGUGCCCGAACGACCAGACCGUA
CUGGCUAACUAUAACGGUCCUAAGGUAGCGAAGAACGAACAAGUUAUCGA
CGGCUGCUGCUGGCGCUGCGAUACCAAAGUUGUAGGGAUAACAGGGUAAU
AACGUAAAGAGAUCCCGCAGUGGUUUAUCAAAAUCACUGCUUACGCUGAC
UAACUAUAACGGUCCUAAGGUAGCGAAUUGCAGCUCAACGAUCUGGAUAN
NNNNN
RNA_3:
[SEQ ID NO: 146]
GNNNNNCAGCGUUCGCUACAGCGUUCGCUAUAACUAUAACGGUCCUAAGG
UAGCGAAAUGCAAGAGCAAUACCGCCCGGAAGAGAUAGAAUCCAAAGUAC
AGCUUCAUAGGGAUAACAGGGUAAUUUGGGAUGAGAAGCGCACAUUUGAA
GUAACCGAAGACGAGAGCAAAGAGAUAACUAUAACGGUCCUAAGGUAGCG
AAAGUAUUACUGCCUGUCUAUGCUUCCCUAUCCUUCUGGUCGACUACACA
UGUAGGGAUAACAGGGUAAUGGCCACGUACGUAACUACACCAUCGGUGAC
GUGAUCGCCCGCUACCAGCGUAACUAUAACGGUCCUAAGGUAGCGAAUAU
GCUGGGCAAAAACGUCCUGCAGCCGAUCGGCUGGGACGCGUUUGGUCUAG
GGAUAACAGGGUAAUUGCCUGCGGAAGGCGCGGCGGUGAAAAACAACACC
GCUCCGGCACCGUGGUAACUAUAACGGUCCUAAGGUAGCGAAACGUACGA
CAACAUCGCGUAUAUGAAAAACCAGCUCAAAAUGCUGGGCUUUAGGGAUA
ACAGGGUAAUUGGUUAUGACUGGAGCCGCGAGCUGGCAACCUGUACGCCG
GAAUACUACCUAACUAUAACGGUCCUAAGGUAGCGAAGUUGGGAACAGAA
AUUCUUCACCGAGCUGUAUAAAAAAGGCCUGGUAUAUUAGGGAUAACAGG
GUAAUAAGAAGACUUCUGCGGUCAACUGGUGCCCGAACGACCAGACCGUA
CUGGCUAACUAUAACGGUCCUAAGGUAGCGAAGAACGAACAAGUUAUCGA
CGGCUGCUGCUGGCGCUGCGAUACCAAAGUUGUAGGGAUAACAGGGUAAU
AACGUAAAGAGAUCCCGCAGUGNNNNNN
RNA_4:
[SEQ ID NO: 147]
GNNNNNCAGCGUUCGCUACAGCGUUCGCUAUAACUAUAACGGUCCUAAGG
UAGCGAAAUGCAAGAGCAAUACCGCCCGGAAGAGAUAGAAUCCAAAGUAC
AGCUUCAUAGGGAUAACAGGGUAAUUUGGGAUGAGAAGCGCACAUUUGAA
GUAACCGAAGACGAGAGCAAAGAGAUAACUAUAACGGUCCUAAGGUAGCG
AAAGUAUUACUGCCUGUCUAUGCUUCCCUAUCCUUCUGGUCGACUACACA
UGUAGGGAUAACAGGGUAAUGGCCACGUACGUAACUACACCAUCGGUGAC
GUGAUCGCCCGCUACCAGCGUAACUAUAACGGUCCUAAGGUAGCGAAUAU
GCUGGGCAAAAACGUCCUGCAGCCGAUCGGCUGGGACGCGUUUGGUCUAG
GGAUAACAGGGUAAUUGCCUGCGGAAGGCGCGGCGGUGAAAAACAACACC
GCUCCGGCACCGUGGUAACUAUAACGGUCCUAAGGUAGCGAAACGUACGA
CAACAUCGCGUAUAUGAAAAACCAGCUCAAAAUGCUGGGCUUUAGGGAUA
ACAGGGUAAUUGGUUAUGACUGGAGCCGCGAGCUGGCAACCUGUACGCCG
GAAUACUACCUAACUAUAACGGUCCUAAGGUAGCGAAGUUGGGAACAGAA
AUUCUUCACCGAGCUGUAUAAAAAAGGCCUGGUAUAUUAGGGAUAACAGG
GUAAUAAGAAGACUUCUGCGGUCAACUGGUGCCCGAACGACCAGACCGUA
CUGGCUAACUAUAACGGUCCUAAGGUAGCGAAGAACGAACAAGUUAUCGA
CGGCNNNNNN
RNA_5:
[SEQ ID NO: 148]
GNNNNNCAGCGUUCGCUACAGCGUUCGCUAUAACUAUAACGGUCCUAAGG
UAGCGAAAUGCAAGAGCAAUACCGCCCGGAAGAGAUAGAAUCCAAAGUAC
AGCUUCAUAGGGAUAACAGGGUAAUUUGGGAUGAGAAGCGCACAUUUGAA
GUAACCGAAGACGAGAGCAAAGAGAUAACUAUAACGGUCCUAAGGUAGCG
AAAGUAUUACUGCCUGUCUAUGCUUCCCUAUCCUUCUGGUCGACUACACA
UGUAGGGAUAACAGGGUAAUGGCCACGUACGUAACUACACCAUCGGUGAC
GUGAUCGCCCGCUACCAGCGUAACUAUAACGGUCCUAAGGUAGCGAAUAU
GCUGGGCAAAAACGUCCUGCAGCCGAUCGGCUGGGACGCGUUUGGUCUAG
GGAUAACAGGGUAAUUGCCUGCGGAAGGCGCGGCGGUGAAAAACAACACC
GCUCCGGCACCGUGGUAACUAUAACGGUCCUAAGGUAGCGAAACGUACGA
CAACAUCGCGUAUAUGAAAAACCAGCUCAAAAUGCUGGGCUUUAGGGAUA
ACAGGGUAAUUGGUUAUGACUGGAGCCGCGAGCUGGCAACCUGUACGCCG
GAAUACUACCUAACUAUAACGGUCCUAAGGUAGCGAAGUUGGGAACAGAA
AUUCUUCACCGAGCUGUAUAAAAAAGGCCUGGUAUAUUAGGGAUAACAGG
GUAAUAAGAAGACUUCUGCGGUCAACUNNNNNN
RNA_6:
[SEQ ID NO: 149]
GNNNNNCAGCGUUCGCUACAGCGUUCGCUAUAACUAUAACGGUCCUAAGG
UAGCGAAAUGCAAGAGCAAUACCGCCCGGAAGAGAUAGAAUCCAAAGUAC
AGCUUCAUAGGGAUAACAGGGUAAUUUGGGAUGAGAAGCGCACAUUUGAA
GUAACCGAAGACGAGAGCAAAGAGAUAACUAUAACGGUCCUAAGGUAGCG
AAAGUAUUACUGCCUGUCUAUGCUUCCCUAUCCUUCUGGUCGACUACACA
UGUAGGGAUAACAGGGUAAUGGCCACGUACGUAACUACACCAUCGGUGAC
GUGAUCGCCCGCUACCAGCGUAACUAUAACGGUCCUAAGGUAGCGAAUAU
GCUGGGCAAAAACGUCCUGCAGCCGAUCGGCUGGGACGCGUUUGGUCUAG
GGAUAACAGGGUAAUUGCCUGCGGAAGGCGCGGCGGUGAAAAACAACACC
GCUCCGGCACCGUGGUAACUAUAACGGUCCUAAGGUAGCGAAACGUACGA
CAACAUCGCGUAUAUGAAAAACCAGCUCAAAAUGCUGGGCUUUAGGGAUA
ACAGGGUAAUUGGUUAUGACUGGAGCCGCGAGCUGGCAACCUGUACGCCG
GAAUACUACCUAACUAUAACGGUCCUAAGGUAGCGAAGUUGGGAACAGAA
AUUCUUCACNNNNNN
RNA_7:
[SEQ ID NO: 150]
GNNNNNCAGCGUUCGCUACAGCGUUCGCUAUAACUAUAACGGUCCUAAGG
UAGCGAAAUGCAAGAGCAAUACCGCCCGGAAGAGAUAGAAUCCAAAGUAC
AGCUUCAUAGGGAUAACAGGGUAAUUUGGGAUGAGAAGCGCACAUUUGAA
GUAACCGAAGACGAGAGCAAAGAGAUAACUAUAACGGUCCUAAGGUAGCG
AAAGUAUUACUGCCUGUCUAUGCUUCCCUAUCCUUCUGGUCGACUACACA
UGUAGGGAUAACAGGGUAAUGGCCACGUACGUAACUACACCAUCGGUGAC
GUGAUCGCCCGCUACCAGCGUAACUAUAACGGUCCUAAGGUAGCGAAUAU
GCUGGGCAAAAACGUCCUGCAGCCGAUCGGCUGGGACGCGUUUGGUCUAG
GGAUAACAGGGUAAUUGCCUGCGGAAGGCGCGGCGGUGAAAAACAACACC
GCUCCGGCACCGUGGUAACUAUAACGGUCCUAAGGUAGCGAAACGUACGA
CAACAUCGCGUAUAUGAAAAACCAGCUCAAAAUGCUGGGCUUUAGGGAUA
ACAGGGUAAUUGGUUAUGACUGGAGCCGCGAGNNNNNN
RNA_8:
[SEQ ID NO: 151]
GNNNNNCAGCGUUCGCUACAGCGUUCGCUAUAACUAUAACGGUCCUAAGG
UAGCGAAAUGCAAGAGCAAUACCGCCCGGAAGAGAUAGAAUCCAAAGUAC
AGCUUCAUAGGGAUAACAGGGUAAUUUGGGAUGAGAAGCGCACAUUUGAA
GUAACCGAAGACGAGAGCAAAGAGAUAACUAUAACGGUCCUAAGGUAGCG
AAAGUAUUACUGCCUGUCUAUGCUUCCCUAUCCUUCUGGUCGACUACACA
UGUAGGGAUAACAGGGUAAUGGCCACGUACGUAACUACACCAUCGGUGAC
GUGAUCGCCCGCUACCAGCGUAACUAUAACGGUCCUAAGGUAGCGAAUAU
GCUGGGCAAAAACGUCCUGCAGCCGAUCGGCUGGGACGCGUUUGGUCUAG
GGAUAACAGGGUAAUUGCCUGCGGAAGGCGCGGCGGUGAAAAACAACACC
GCUCCGGCACCGUGGUAACUAUAACGGUCCUAAGGUAGCGAAACGUACGA
CAACAUCGCGUAUANNNNNN
RNA_9:
[SEQ ID NO: 152]
GNNNNNCAGCGUUCGCUACAGCGUUCGCUAUAACUAUAACGGUCCUAAGG
UAGCGAAAUGCAAGAGCAAUACCGCCCGGAAGAGAUAGAAUCCAAAGUAC
AGCUUCAUAGGGAUAACAGGGUAAUUUGGGAUGAGAAGCGCACAUUUGAA
GUAACCGAAGACGAGAGCAAAGAGAUAACUAUAACGGUCCUAAGGUAGCG
AAAGUAUUACUGCCUGUCUAUGCUUCCCUAUCCUUCUGGUCGACUACACA
UGUAGGGAUAACAGGGUAAUGGCCACGUACGUAACUACACCAUCGGUGAC
GUGAUCGCCCGCUACCAGCGUAACUAUAACGGUCCUAAGGUAGCGAAUAU
GCUGGGCAAAAACGUCCUGCAGCCGAUCGGCUGGGACGCGUUUGGUCUAG
GGAUAACAGGGUAAUUGCCUGCGGAAGGCGCGGCGGUNNNNNN
RNA_10:
[SEQ ID NO: 153]
GNNNNNCAGCGUUCGCUACAGCGUUCGCUAUAACUAUAACGGUCCUAAGG
UAGCGAAAUGCAAGAGCAAUACCGCCCGGAAGAGAUAGAAUCCAAAGUAC
AGCUUCAUAGGGAUAACAGGGUAAUUUGGGAUGAGAAGCGCACAUUUGAA
GUAACCGAAGACGAGAGCAAAGAGAUAACUAUAACGGUCCUAAGGUAGCG
AAAGUAUUACUGCCUGUCUAUGCUUCCCUAUCCUUCUGGUCGACUACACA
UGUAGGGAUAACAGGGUAAUGGCCACGUACGUAACUACACCAUCGGUGAC
GUGAUCGCCCGCUACCAGCGUAACUAUAACGGUCCUAAGGUAGCGAAUAU
GCUGGGCAAAAACGUCCUGNNNNNN

FIG. 6

Primers used to amplify carrier molecules. The same forward primer is used to create PCR templates for all carrier molecules. T7 promoter sequence is underlined: PCR_GN5_f1:

[SEQ ID NO: 154]
TAATACGACTCACTATAGNNNNNCAGCGTTCGCTA

FIG. 7

A) PCR conditions used to create carrier templates. B) Carrier combinations tested in SLIC-CAGE.^{a} Proportions of each carrier used are given in FIG. 8.

FIG. 8

A) Carrier molecule quantities used in SLIC-CAGE. Provides approximately 50 μg of the carrier mix 0.3-1 kb (44 μg of uncapped and 5 μg of capped). B) Primer sequences for qPCR used to estimate the ratio of target library and the leftover carrier. C) Real-time qPCR cycling conditions. D) PCR amplification conditions. ^{a}x corresponds to Ct value obtained in qPCR with adapter_f1 and adapter_r1 primers.

FIG. 9

Number of PCR cycles used to amplify SLIC-CAGE and nanoCAGE libraries. ^{a}reference nAnT-iCAGE sample diluted 100-fold and PCR amplified 13 cycles using adapter_f1 and adapter_r1 primers.

FIG. 10

SLIC-CAGE, nAnT-iCAGE and nanoCAGE mapping efficiency.

FIG. 11

SLIC-CAGE carrier leftover.

FIG. 12

CTSS and tag cluster in SLIC-CAGE and nAnTi-CAGE.

FIG. 13

A) CTSS and tag cluster identification in nanocage. B) CTSS and tag cluster identification in nanocage. ^{a}Number of alignment mismatches at the 1st and 2nd nucleotide position in nanoCAGE tags. ^{b}Number of GG dinucleotides at 1st and 2nd position in nanoCAGE tags, flagged as mismatches in the alignment.

FIG. 14

Template switching oligonucleotides used in nanoCAGE. ^{a}TSO sequences are from Poulain, S. et al. NanoCAGE: A Method for the Analysis of Coding and Noncoding 5′-Capped Transcriptomes. Methods Mol Biol 1543, 57-109 (2017).

FIG. 15

Design and test of carrier molecules. (a) Schematics of the recombinant plasmid with the synthetic carrier gene. (b) Workflow for preparation of the carrier molecules with embedded I-CeuI and I-SceI recognition sites. First, the DNA template for in vitro transcription is produced using PCR amplification with a common forward primer (PCR_GN5_f1) and a variety of reverse primers (PCR_N6_r1-r10), to synthesise PCR templates of different lengths (931-351 nucleotides, Supplementary Table 2). The forward primer contains the T7-promoter sequence, and a GN₅ sequence (N—random nucleotide). The reverse primer dictates the length of the final carrier and introduces random nucleotides at the 3′end of carrier molecules (N₆). After PCR-amplification, the templates are gel-purified and the carrier molecules synthesised using run-off in vitro transcription. Carriers are then purified and a portion of it capped, followed by purification. (c-h) Test of various carrier mixes added to 100 ng of S. cerevisiae total RNA. Pearson correlation of the libraries constructed using 100 ng of S. cerevisiae total RNA and (c) no carrier added, (d) mix 1: mix of 931 nucleotides capped (0.5 μg) and 931 nucleotides (4.4 μg) uncapped carrier, (e) mix 2: mix of 351-931 nucleotides capped (0.5 μg) and 351-931 nucleotides (4.4 μg) uncapped carrier, replicate 1, (f) mix 2: same as in (e), replicate 2, (g) mix 3: 931 nucleotides capped (0.5 μg) carrier, (h) mix 4: 351-931 nucleotides capped (0.5 μg) carrier. All carrier mixes are presented in detail in the Supplementary Table 4 and 5. (i) Pearson correlation of two nAnT-iCAGE technical replicates constructed using 5 μg of total S. cerevisiae RNA. (j) Genomic locations of tag clusters identified in carrier test SLIC-CAGE libraries and the reference nAnT-iCAGE library. (k) Distribution of tag cluster interquantile widths in carrier test SLIC-CAGE libraries and the reference nAnT-iCAGE library. (l) Nucleotide composition of all CTSSs identified in carrier test SLIC-CAGE libraries and in the reference nAnT-iCAGE library. (m) Dinucleotide composition of all CTSSs (left panel) or dominant CTSSs (right panel) identified in carrier test SLIC-CAGE libraries and in the reference nAnT-iCAGE library. Both panels are ordered from the most to least used dinucleotide in the reference nAnT-iCAGE.

FIG. 16

Performance comparison of SLIC-CAGE and nanoCAGE libraries. Pearson correlation coefficients of (a) SLIC-CAGE libraries constructed from 1-100 ng of S. cerevisiae total RNA and corresponding nAnT-iCAGE libraries (b) nanoCAGE libraries constructed from 5-500 ng of S. cerevisiae total RNA and the nAnT-iCAGE libraries (c) SLIC-CAGE libraries constructed from 5-100 ng of M. musculus total RNA and the reference nAnT-iCAGE library. (d-f) Genomic locations of tag clusters identified in (d) S. cerevisiae SLIC-CAGE libraries and the reference nAnT-iCAGE library, (e) S. cerevisiae nanoCAGE libraries and the reference nAnT-iCAGE library, (f) M. musculus SLIC-CAGE libraries and the reference nAnT-iCAGE library. (g-i) Nucleotide composition of all CTSSs identified in (g) S. cerevisiae SLIC-CAGE libraries, (h) S. cerevisiae nanoCAGE libraries, (i) M. musculus SLIC-CAGE libraries. (j-l) Dinucleotide composition of all CTSSs identified in (j) S. cerevisiae SLIC-CAGE libraries, (k) S. cerevisiae nanoCAGE libraries, (l) M. musculus SLIC-CAGE libraries. All panels are ordered from the most to the least used dinucleotide in the reference nAnT-iCAGE. (m-o) Dinucleotide composition of dominant CTSSs identified in (m) S. cerevisiae SLIC-CAGE libraries, (n) S. cerevisiae nanoCAGE libraries, (o) M. musculus SLIC-CAGE libraries. All panels are ordered from the most to the least used dominant CTSS dinucleotide in the reference nAnT-iCAGE.

FIG. 17

Distribution of tag cluster interquantile widths in (a) SLIC-CAGE libraries prepared from 1-100 ng of S. cerevisiae total RNA in comparison with the nAnT-iCAGE and PCR amplified nAnT-iCAGE library (diluted in water 1:100 and PCR amplified—13 cycles). (b) SLIC-CAGE libraries prepared from 5-100 ng of M. musculus total RNA in comparison with nAnT-iCAGE. (c) nanoCAGE libraries prepared from 5-500 ng of S. cerevisiae total RNA in comparison nAnT-iCAGE.

FIG. 18

Assessment of positional accuracy in S. cerevisiae SLIC-CAGE libraries prepared from various amounts of total RNA (a) 1 ng, (b) 2 ng, (c) 5 ng, (d) 10 ng, replicate 1, (e) 10 ng, replicate 2, (f) 25 ng, (g) 50 ng, (h) 100 ng, or (i) nAnT-iCAGE library prepared from 5 μg of total RNA, diluted 1:100 and PCR amplified. Left panels: heatmaps represent log 10(TPM ratio), where the ratio is defined as nAnT-iCAGE TPM value divided with the corresponding SLIC-CAGE TPM value for each CTSS identified in both libraries. The horizontal lines separate four expression level (TPM) quantiles, with the lowest expression quantile on top, and the highest at the bottom of the heatmap. Within each quantile, the sequences are ordered from the highest to the lowest overall TPM ratio values per tag cluster in each SLIC-CAGE library. Middle panels: heatmaps represent the log 10 TPM value of the CTSS present in the nAnT-iCAGE and absent from the SLIC-CAGE library, or the −log 10 TPM value of the CTSS present in the SLIC-CAGE library and absent from the nAnT-iCAGE library. Ordering is the same as explained for left panels. Right panels: coverage of CTSSs present in the reference nAnT-iCAGE library, centred on the dominant CTSS identified in the SLIC-CAGE library with ordering as in the left panels.

FIG. 19

Assessment of positional accuracy in M. musculus SLIC-CAGE libraries prepared from various amounts of total RNA (a) 5 ng, (b) 10 ng, (c) 25 ng, (d) 50 ng or (e) 100 ng. Left panels: heatmaps represent log 10(TPM ratio), where the ratio is defined as nAnT-iCAGE TPM value divided with the corresponding SLIC-CAGE TPM value for each CTSS identified in both libraries. The horizontal lines separate four expression level (TPM) quantiles, with the lowest expression quantile on top, and the highest at the bottom of the heatmap. Within each quantile, the sequences are ordered from the highest to the lowest overall TPM ratio values per tag cluster in each SLIC-CAGE library. Middle panels: heatmaps represent the log 10 TPM value of the CTSS present in the nAnT-iCAGE and absent from the SLIC-CAGE library, or the −log 10 TPM value of the CTSS present in the SLIC-CAGE library and absent from the nAnT-iCAGE library. Ordering is the same as explained for left panels. Right panels: coverage of CTSSs present in the reference nAnT-iCAGE library, centred on the dominant CTSS identified in the SLIC-CAGE library with ordering as in the left panels.

FIG. 20

Dinucleotide composition of dominant CTSSs identified in nanoCAGE libraries derived from 5-500 ng of S. cerevisiae total RNA and compared with the nAnT-iCAGE library (derived from 5 μg of total RNA). Dominant CTSSs are split according to genomic locations.

FIG. 21

Dinucleotide composition of dominant CTSSs identified in nanoCAGE libraries derived from 5-500 ng of S. cerevisiae total RNA and compared with the nAnT-iCAGE library (derived from 5 μg of total RNA). Dominant CTSSs are split according their expression (TPM) values into quartiles (Q1—the lowest 25%, Q4—the highest 25%).

FIG. 22

Assessment of positional accuracy in S. cerevisiae nanoCAGE libraries prepared from various amounts of total RNA (a) 5 ng, (b) 10 ng, replicate 1 (c) 10 ng, replicate 2 (d) 25 ng, replicate 1 (e) 25 ng, replicate 2 (f) 50 ng, (g) 500 ng, replicate 1 (h) 500 ng, replicate 2 or (i) nAnT-iCAGE library prepared from 5 μg of total RNA, replicate 1. Left panels: heatmaps represent log 10(TPM ratio), where the ratio is defined as nAnT-iCAGE TPM value divided with the corresponding SLIC-CAGE TPM value for each CTSS identified in both libraries. The horizontal lines separate four expression level (TPM) quantiles, with the lowest expression quantile on top, and the highest at the bottom of the heatmap. Within each quantile, the sequences are ordered from the highest to the lowest overall TPM ratio values per tag cluster in each nanoCAGE library. Middle panels: heatmaps represent the log 10 TPM value of the CTSS present in the nAnT-iCAGE and absent from the nanoCAGE library, or the −log 10 TPM value of the CTSS present in the nanoCAGE library and absent from the nAnT-iCAGE library. Ordering is the same as explained for left panels. Right panels: coverage of CTSSs present in the reference nAnT-iCAGE library, centred on dominant CTSS identified in the nanoCAGE library with ordering as in the left panels.

FIG. 23

Separation of sharp and broad promoters/tag clusters in M. musculus SLIC-CAGE libraries. (a) Number of sharp or broad tag clusters (y-axis) in dependence of the interquantile width threshold (x-axis). The white dashed vertical line marks the chosen empirical threshold for separating sharp and broad tag clusters/promoters (sharp have interquantile width <=3 and broad >3). (b) Average AA/AT/TA/TT dinucleotide relative frequency in sharp or broad promoters identified in SLIC-CAGE or nAnT-iCAGE libraries. (c) Comparison of TATA-box density in SLIC-CAGE and nAnT-iCAGE libraries. Promoter regions are scanned using a minimum of 80^{th} percentile match to the TATA-box pwm, centred on the dominant TSS and ordered by interquantile width with the sharpest promoters on top of the heatmap, and broadest at the bottom. The horizontal black line separates sharp and broad promoters, defined in (a). (d) TATA-box relative frequency in sharp or broad promoters.

FIG. 24

Pattern discovery in M. musculus SLIC-CAGE libraries. Comparison of CTSS coverage, TA dinucleotide density, GC dinucleotide density, H3K4me3 coverage, CpG islands coverage in SLIC-CAGE libraries prepared from (a) 5 ng, (b) 10 ng, (c) 25 ng, (d) 50 ng or (e) 100 ng of total RNA and nAnTi-CAGE library prepared from (f) 5 μg of total RNA. Windows are centred on the dominant CTSSs identified in SLIC-CAGE or nAnT-iCAGE libraries. Promoter regions are all ordered from sharpest to broadest tag cluster interquantile width. The horizontal line separates sharp and broad promoters (defined by an empirical threshold where interquantile width <=3 defines sharp, and interquantile width >3 defines broad promoters).

FIG. 25

Detailed workflow of SLIC-CAGE protocol steps following carrier degradation.

FIG. 26

Representative HS DNA bioanalyzer traces of libraries prepared form 5 or 10 of M. musculus total RNA after carrier degradation and PCR amplification steps: (a, b) prior to 2^{nd} round of AMPure XP size selection; (c, d) final SLIC-CAGE library after 2^{nd} round of AMPure XP size selection.

FIG. 27

Predicted frequency of cutting by homing endonuclease enzymes in various genomes.

^{a}Saccharomyces cerevisiae genome size: 12,100,000 nucleotides; GC content: 38%
^{b}Drosophila melanogaster genome size: 175,000,000 nucleotides; GC content: 43%
^{c}Mus musculus genome size: 2,700,000,000 nucleotides; GC content: 42%
^{d}Homo sapiens genome size: 3,289,000,000 nucleotides, GC content: 41%
*Perfect sequence matching is taken into account.
**First number in the table is calculated using equal probability of each nucleotide. The second number takes into account the GC content of each organism (therefore it is more accurate).

FIG. 28

Exemplary endonucleases that are considered to be suitable for use in the invention.

### REFERENCES

Poulain, S. et al. NanoCAGE: A Method for the Analysis of Coding and Noncoding 5′-Capped Transcriptomes. Methods Mol Biol 1543, 57-109 (2017).
1. Shiraki, T. et al. Cap analysis gene expression for high-throughput analysis of transcriptional starting point and identification of promoter usage. Proceedings of the National Academy of Sciences of the United States of America 100, 15776-15781 (2003).
2. Smale, S. T. & Kadonaga, J. T. The RNA polymerase II core promoter. Annu Rev Biochem 72, 449-479 (2003).
3. Consortium, F. et al. A promoter-level mammalian expression atlas. Nature 507, 462-470 (2014).
4. Andersson, R. et al. An atlas of active enhancers across human cell types and tissues. Nature 507, 455-461 (2014).
5. Haberle, V. et al. Two independent transcription initiation codes overlap on vertebrate core promoters. Nature 507, 381-385 (2014).
6. Lenhard, B., Sandelin, A. & Carninci, P. Metazoan promoters: emerging characteristics and insights into transcriptional regulation. Nat Rev Genet 13, 233-245 (2012).
7. Haberle, V. & Lenhard, B. Promoter architectures and developmental gene regulation. Semin Cell Dev Biol 57, 11-23 (2016).
8. Consortium, E. P. An integrated encyclopedia of DNA elements in the human genome. Nature 489, 57-74 (2012).
9. Celniker, S. E. et al. Unlocking the secrets of the genome. Nature 459, 927-930 (2009).
10. Kawaji, H., Kasukawa, T., Forrest, A., Carninci, P. & Hayashizaki, Y. The FANTOM5 collection, a data series underpinning mammalian transcriptome atlases in diverse cell types. Sci Data 4, 170113 (2017).
11. Carninci, P. et al. High-efficiency full-length cDNA cloning by biotinylated CAP trapper. Genomics 37, 327-336 (1996).
12. Kodzius, R. et al. CAGE: cap analysis of gene expression. Nat Methods 3, 211-222 (2006).
13. Takahashi, H., Lassmann, T., Murata, M. & Carninci, P. 5′ end-centered expression profiling using cap-analysis gene expression and next-generation sequencing. Nat Protoc 7, 542-561 (2012).
14. Murata, M. et al. Detecting expressed genes using CAGE. Methods Mol Biol 1164, 67-85 (2014).
15. Plessy, C. et al. Linking promoters to functional transcripts in small samples with nanoCAGE and CAGEscan. Nat Methods 7, 528-534 (2010).
16. Poulain, S. et al. NanoCAGE: A Method for the Analysis of Coding and Noncoding 5′-Capped Transcriptomes. Methods Mol Biol 1543, 57-109 (2017).
17. Zhu, Y. Y., Chenchik, A., Li, R., Hsieh, F. Y. & Siebert, P. D. in Genetic Library Construction and Screening: Advanced Techniques and Applications. (eds. R. C. Bird & B. F. Smith) 69-93 (Springer Berlin Heidelberg, Berlin, Heidelberg; 2002).
18. Tang, D. T. et al. Suppression of artifacts and barcode bias in high-throughput transcriptome analyses utilizing template switching. Nucleic acids research 41, e44 (2013).
19. Kivioja, T. et al. Counting absolute numbers of molecules using unique molecular identifiers. Nat Methods 9, 72-74 (2011).
20. Smith, T., Heger, A. & Sudbery, I. UMI-tools: modeling sequencing errors in Unique Molecular Identifiers to improve quantification accuracy. Genome Res 27, 491-499 (2017).
21. Gimble, F. S. & Wang, J. Substrate recognition and induced DNA distortion by the PI-SceI endonuclease, an enzyme generated by protein splicing. J Mol Biol 263, 163-180 (1996).
22. Argast, G. M., Stephens, K. M., Emond, M. J. & Monnat, R. J., Jr. I-PpoI and I-CreI homing site sequence degeneracy determined by random mutagenesis and sequential in vitro enrichment. J Mol Biol 280, 345-353 (1998).
23. Palazzo, A. F. & Lee, E. S. Non-coding RNA: what is functional and what is junk? Front Genet 6, 2 (2015).
24. Carninci, P. et al. Genome-wide analysis of mammalian promoter architecture and evolution. Nat Genet 38, 626-635 (2006).
25. Haberle, V., Forrest, A. R., Hayashizaki, Y., Carninci, P. & Lenhard, B. CAGEr: precise TSS data retrieval and high-resolution promoterome mining for integrative analyses. Nucleic acids research 43, e51 (2015).
26. Burke, T. W. & Kadonaga, J. T. The downstream core promoter element, DPE, is conserved from Drosophila to humans and is recognized by TAFII60 of Drosophila. Genes Dev 11, 3020-3031 (1997).
27. Zajac, P., Islam, S., Hochgerner, H., Lonnerberg, P. & Linnarsson, S. Base preferences in non-templated nucleotide incorporation by MMLV-derived reverse transcriptases. PLoS One 8, e85270 (2013).
28. Ponjavic, J. et al. Transcriptional and structural impact of TATA-initiation site spacing in mammalian core promoters. Genome Biol 7, R78 (2006).
29. Kutach, A. K. & Kadonaga, J. T. The downstream promoter element DPE appears to be as widely used as the TATA box in Drosophila core promoters. Mol Cell Biol 20, 4754-4764 (2000).
30. Segal, E. et al. A genomic code for nucleosome positioning. Nature 442, 772-778 (2006).
31. Zheng, X. et al. Low-Cell-Number Epigenome Profiling Aids the Study of Lens Aging and Hematopoiesis. Cell Rep 13, 1505-1518 (2015).
32. Langmead, B. & Salzberg, S. L. Fast gapped-read alignment with Bowtie 2. Nature Methods 9, 357 (2012).
33. Balwierz, P. J. et al. Methods for analyzing deep sequencing expression data: constructing the human and mouse promoterome with deepCAGE data. Genome Biol 10, R79 (2009).
34. Yu, G., Wang, L. G. & He, Q. Y. ChlPseeker: an R/Bioconductor package for ChIP peak annotation, comparison and visualization. Bioinformatics 31, 2382-2383 (2015).
35. Aken, B. L. et al. The Ensembl gene annotation system. Database (Oxford) 2016 (2016).
36. Wickham, H. Ggplot2: elegant graphics for data analysis. (Springer, New York; 2009).
37. Li, H. et al. The Sequence Alignment/Map format and SAMtools. Bioinformatics 25, 2078-2079 (2009).
38. Lawrence, M., Gentleman, R. & Carey, V. rtracklayer: an R package for interfacing with genome browsers. Bioinformatics 25, 1841-1842 (2009).

### EXAMPLES

### Example 1—Introduction and Development of SLIC-CAGE

The inventors have developed SLIC-CAGE, a Super-Low Input Carrier-CAGE approach that is based on cap-trapper technology and can generate unbiased high-complexity libraries from 5-10 ng of total RNA. Thus far the cap-trapper step has been the limiting factor in the reduction of the amount of required starting material. To facilitate the cap-trapper technology on the nanogram scale, representing capped RNA from as low as hundreds of eukaryotic cells, samples of the total RNA of interest are supplemented with novel pre-designed carrier RNAs. Prior to sequencing, the carrier is efficiently removed from the final library using homing endonucleases that target recognition sites embedded within the sequences of the carrier molecules, leaving only the target mRNA library to be amplified and sequenced. The specificity and the long recognition motifs of homing endonucleases ensure that no sample RNA is degraded in the process.

The inventors have tested and validated SLIC-CAGE on a wide-range of starting material amounts (1-100 ng of total RNA) from Saccharomyces cerevisiae and Mus musculus using the current nAnT-iCAGE protocol, as a gold standard. Additional direct comparison between SLIC-CAGE and the latest nanoCAGE protocol¹⁶ showed that SLIC-CAGE strongly outperforms nanoCAGE in sensitivity, resolution and reproducibility. SLIC-CAGE produced unbiased libraries of higher complexity and quality than nanoCAGE, even when constructed using low total RNA input (5-10 ng compared to 500 ng). Taken together, the inventors demonstrate that SLIC-CAGE enables reliable genome-wide promoter-centric biological discovery and promoter classification using as little as 5-10 ng of total RNA material.

In typical CAGE protocols, the cap-trapper step needs at least 5 μg of total RNA¹⁴ and is therefore the limiting factor. This step has been difficult to scale down as it involves the pull-down of biotinylated capped RNA using streptavidin beads. In such situations, a common biochemical approach to prevent sample loss is the use of carriers; i.e. inert non-interfering molecules to minimize sample loss caused by nonspecific adsorption and to improve specificity in affinity purification steps. However, unless there is a way to selectively remove the carrier afterwards, the carrier signal will dominate the sequenced sample and therefore lead to orders of magnitude of reduced sequencing depth of the sample itself.

To solve this problem and enable profiling of minute amounts of RNA, the inventors have designed carrier RNA that will be similar in size distribution and the percentage of capped RNA to the cellular RNA, but whose cDNA will be possible to selectively degrade without affecting the cDNA originating from the sample.

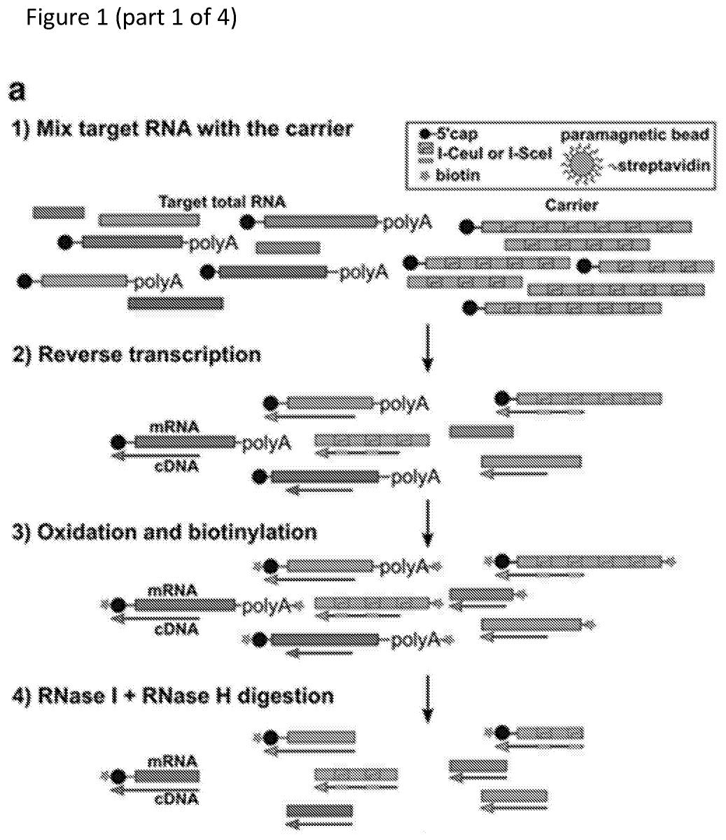

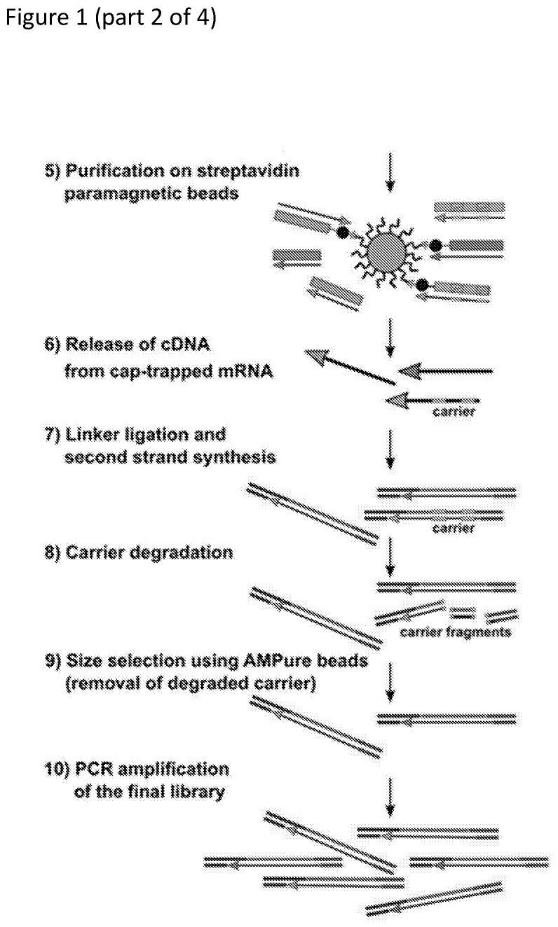

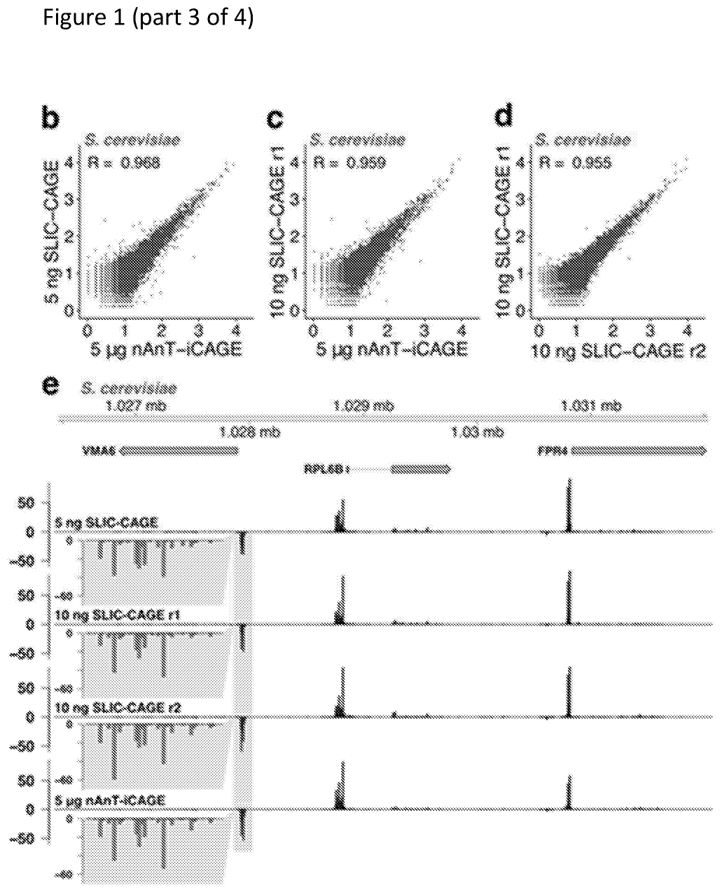

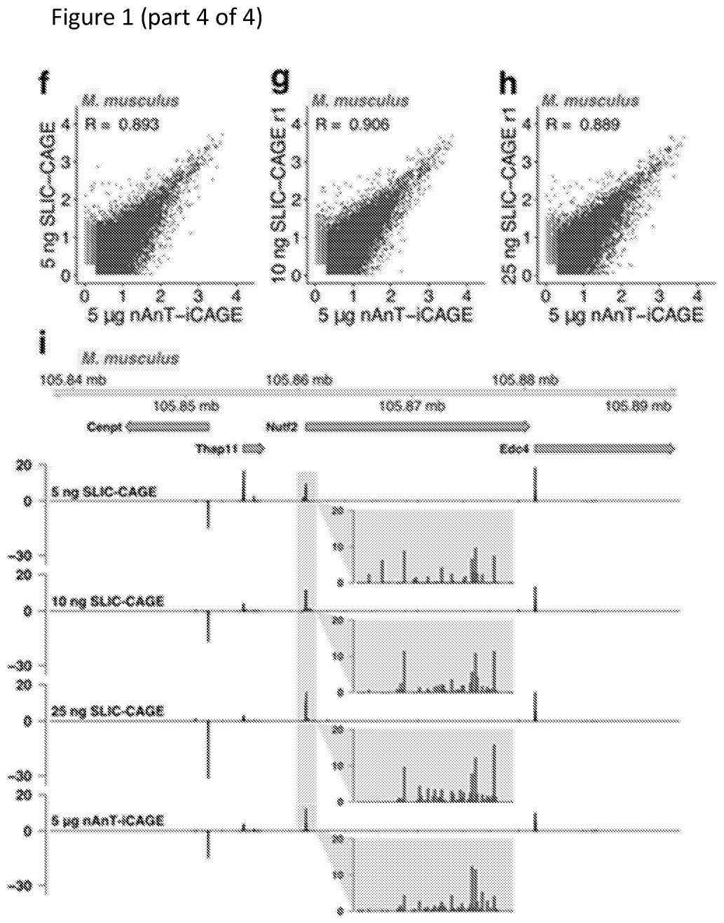

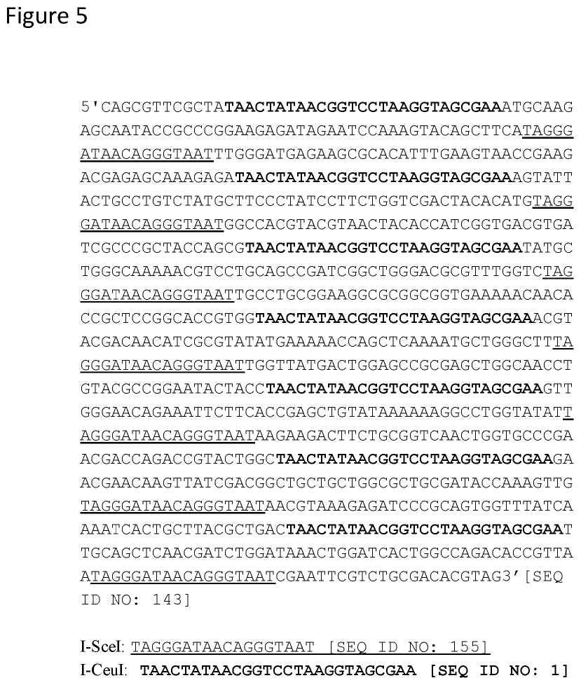

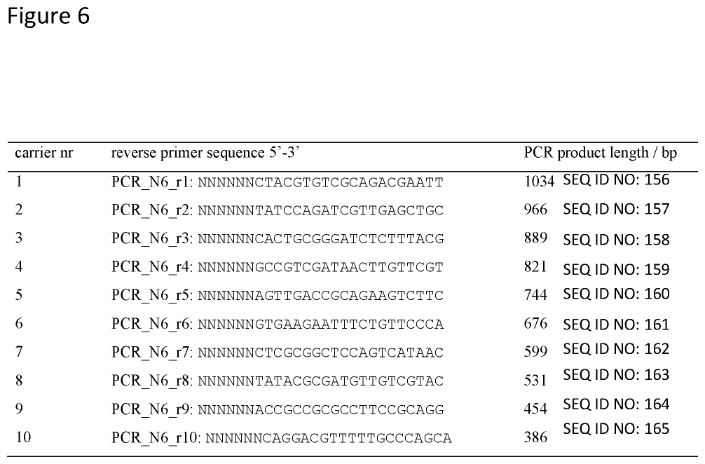

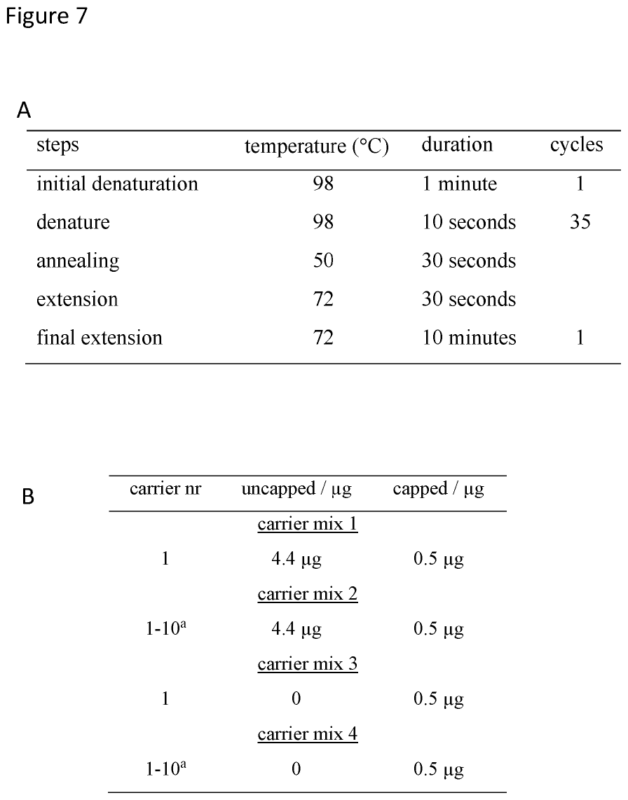

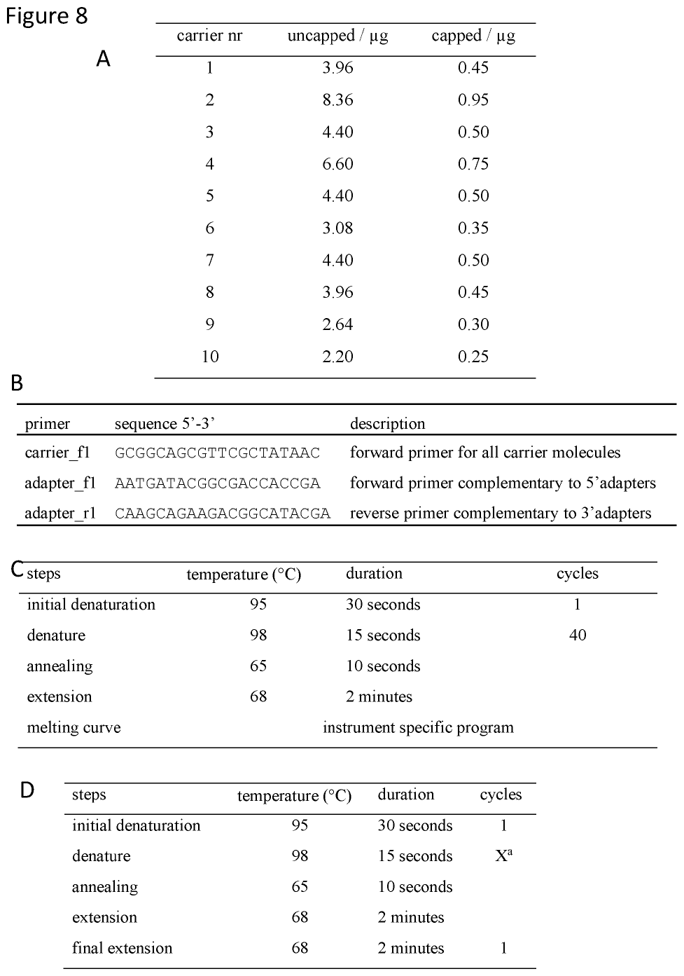

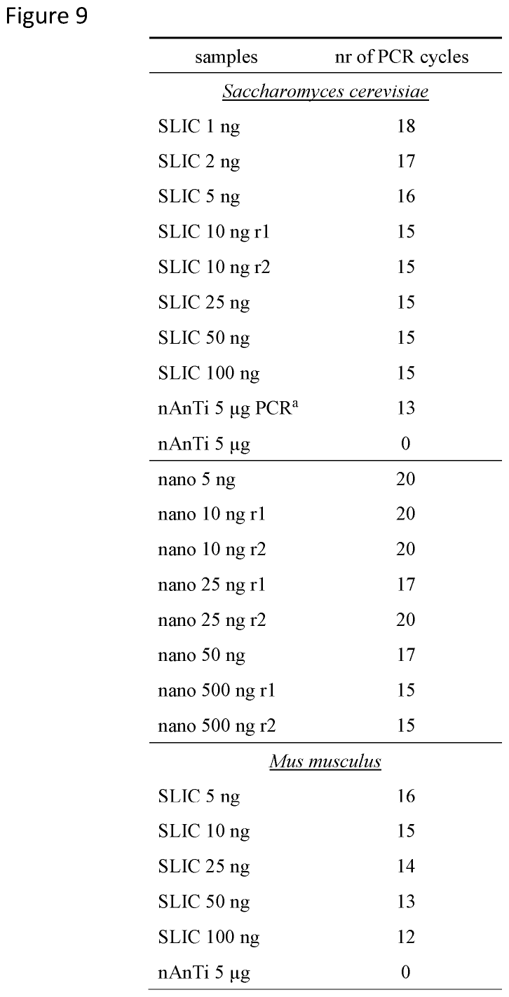

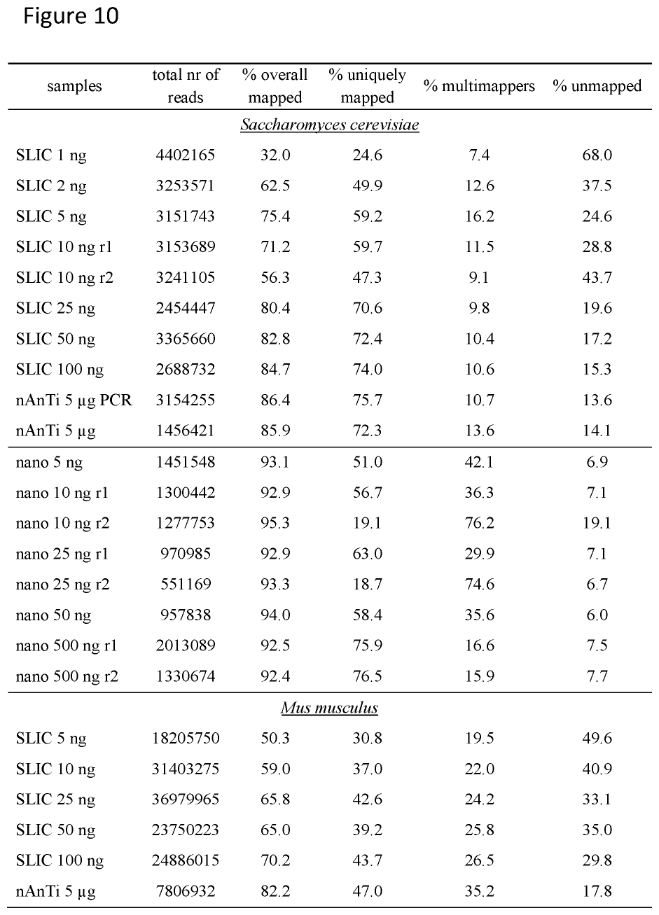

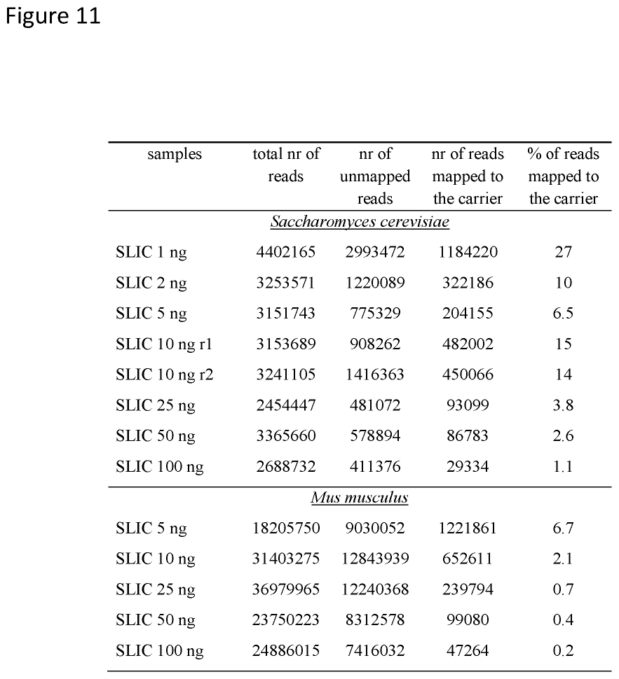

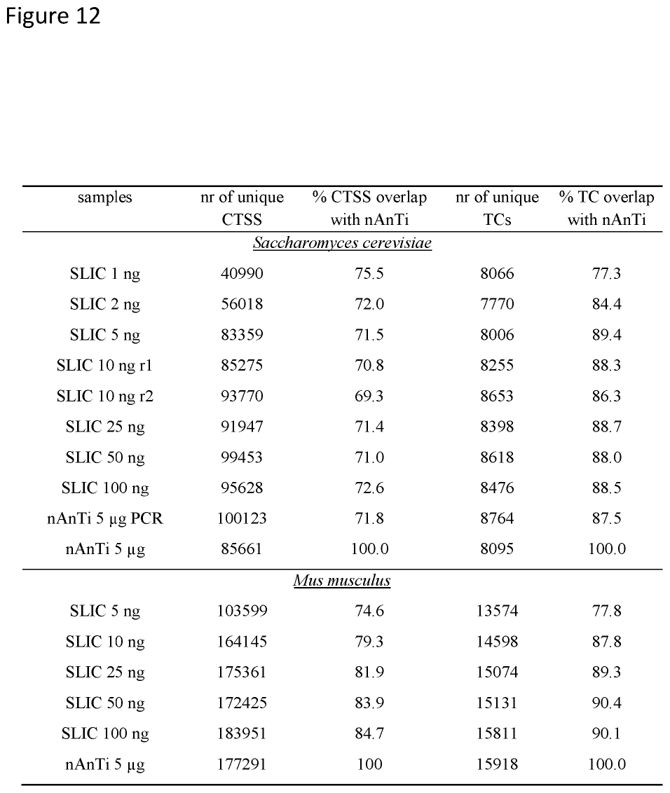

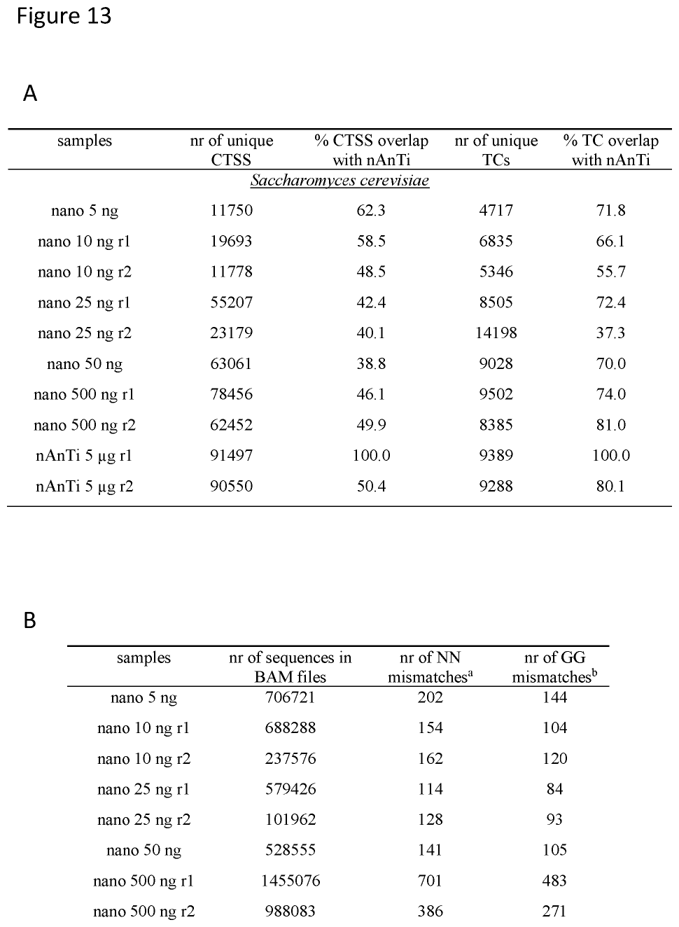

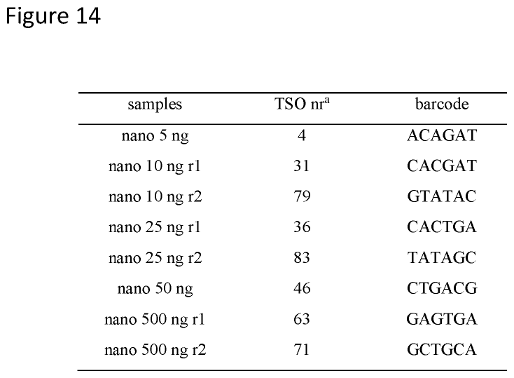

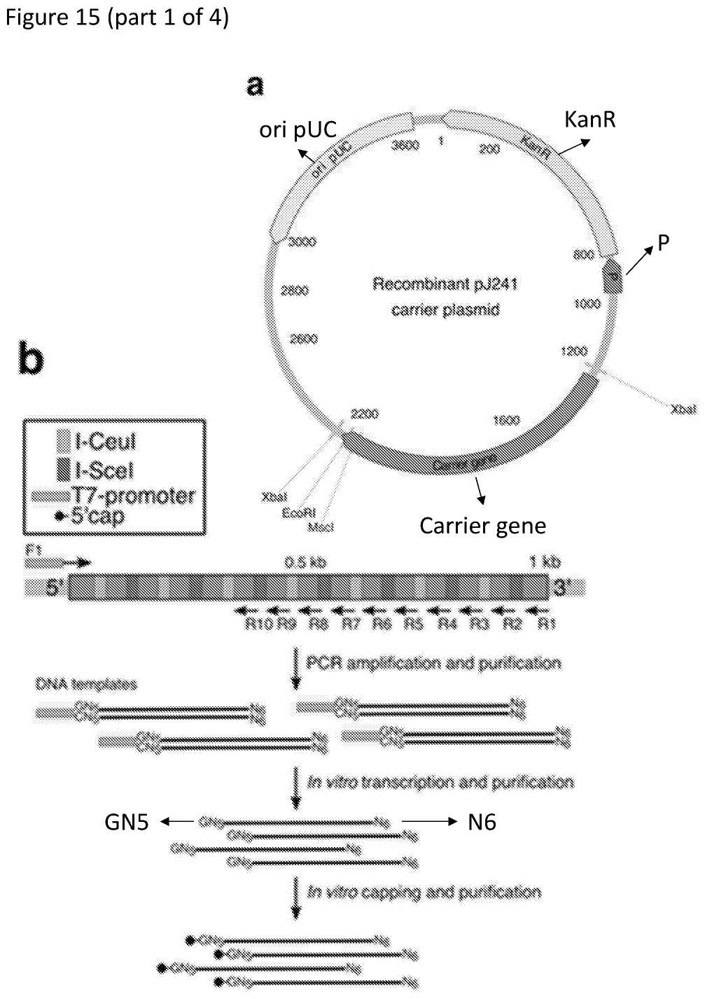

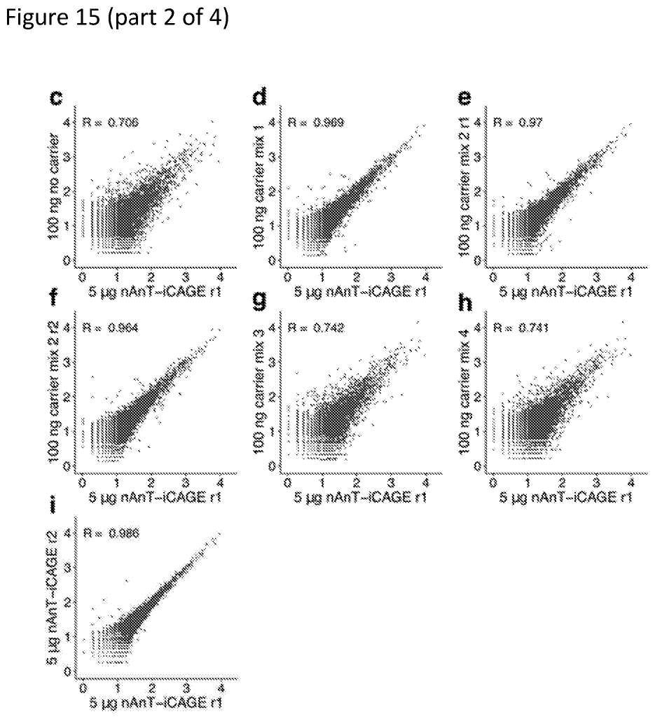

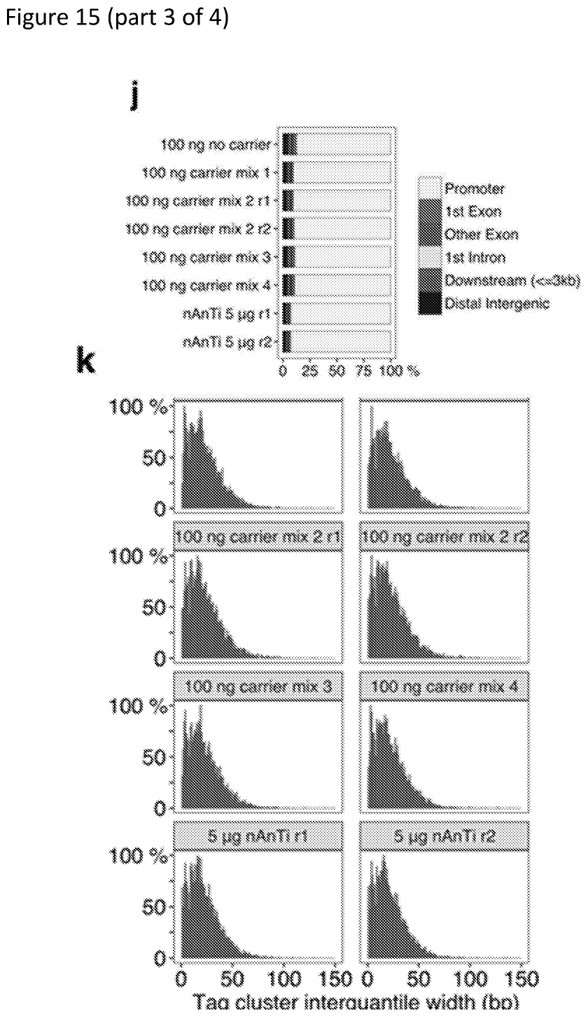

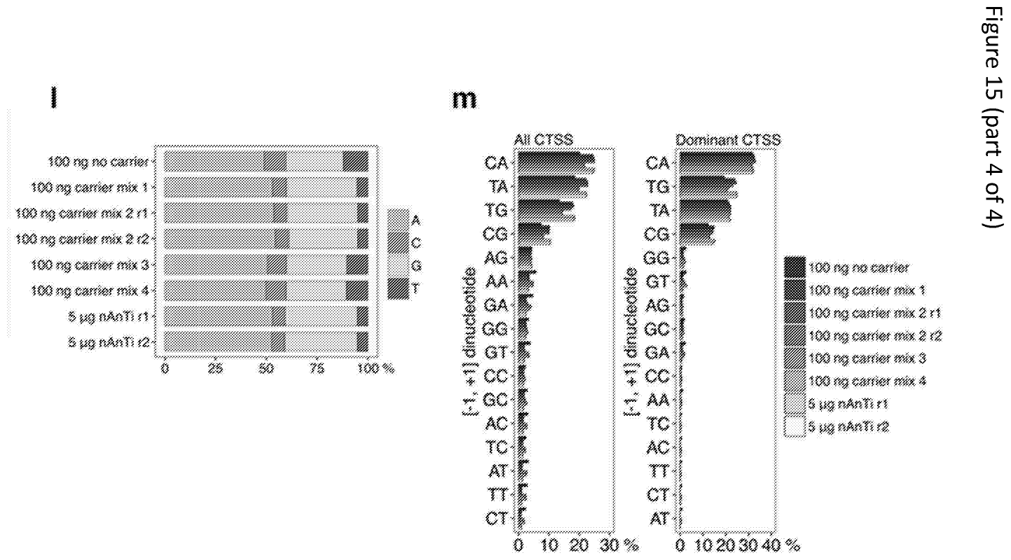

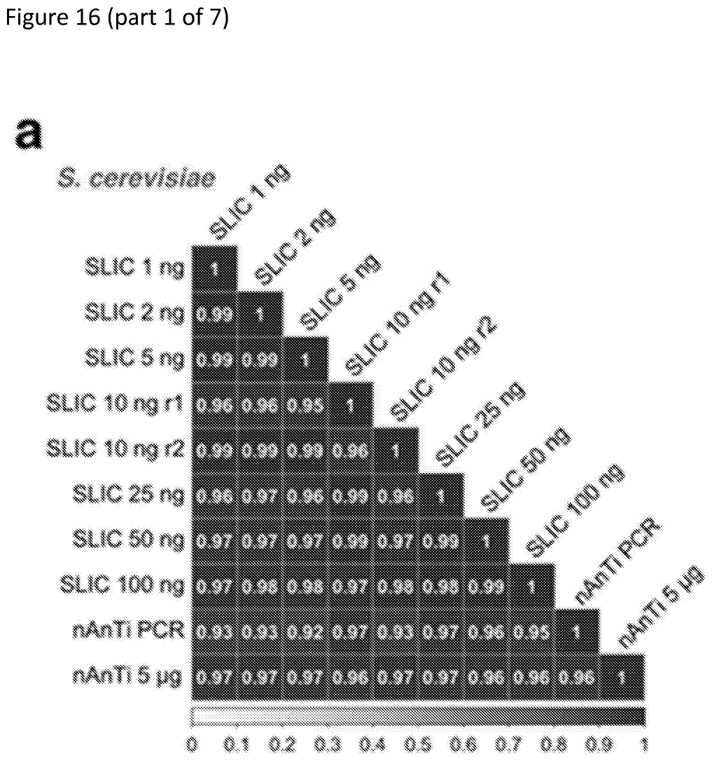

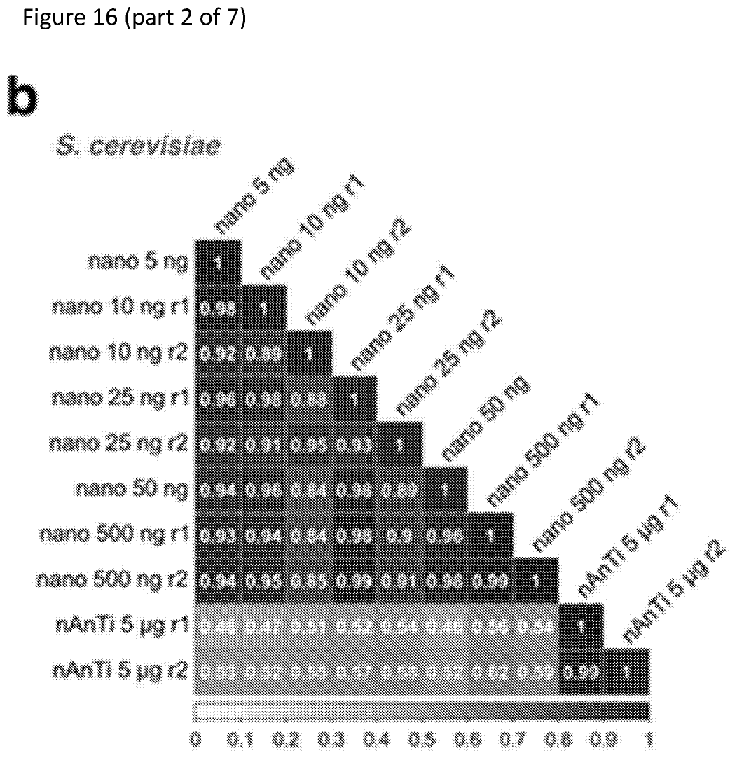

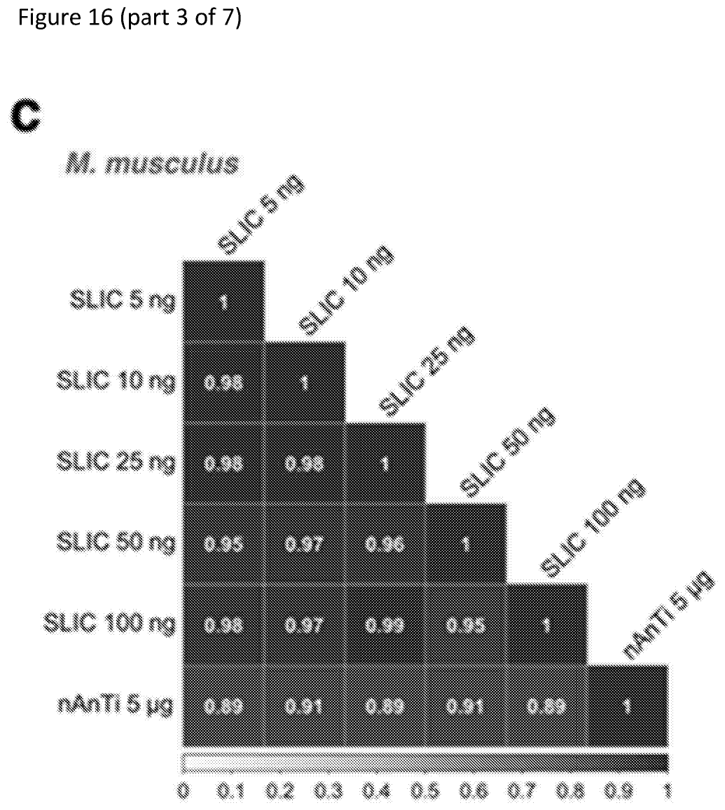

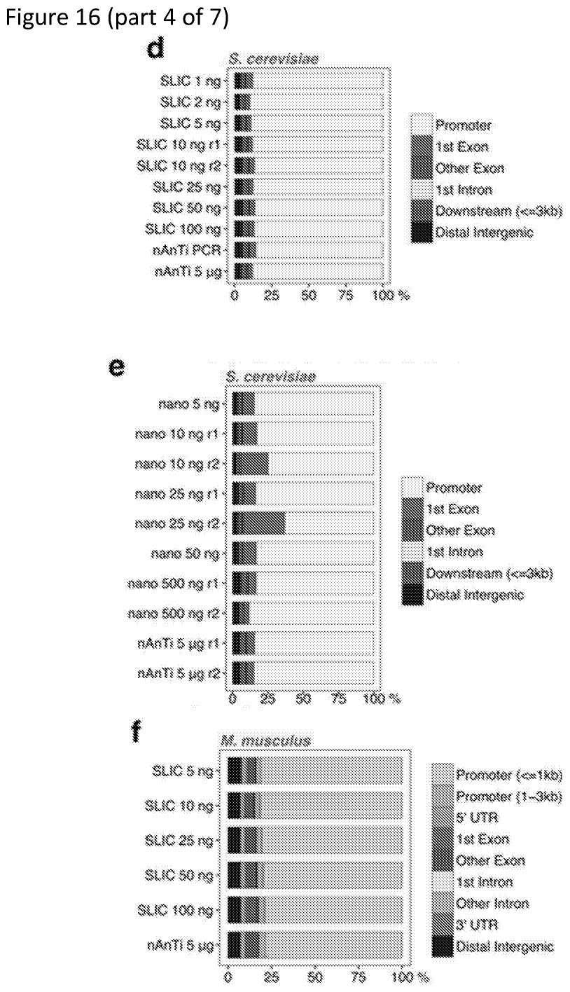

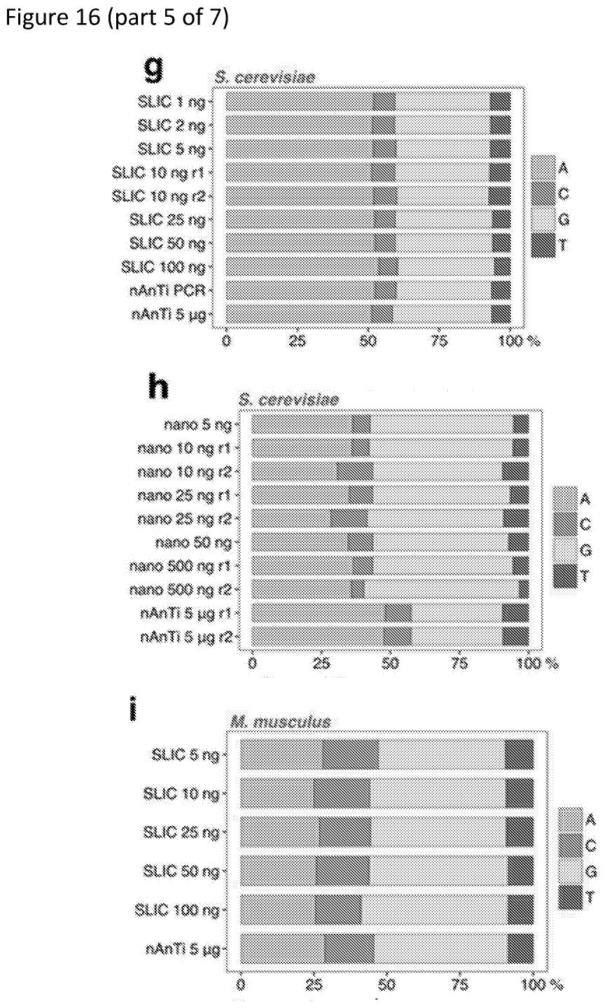

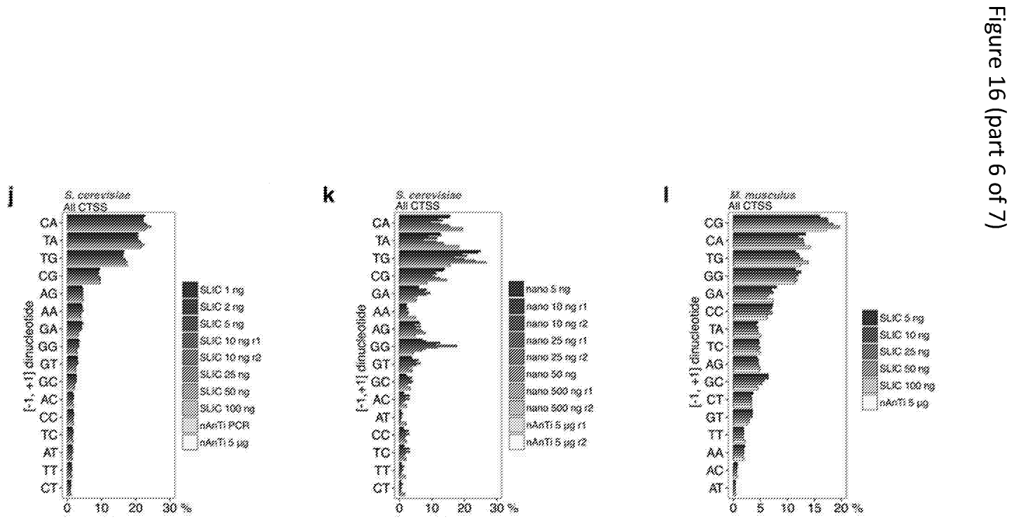

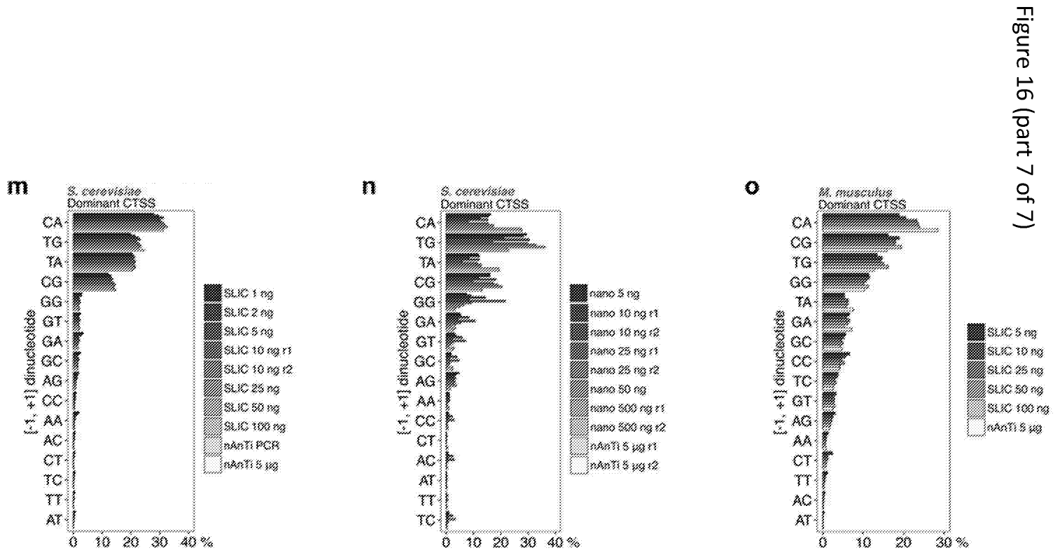

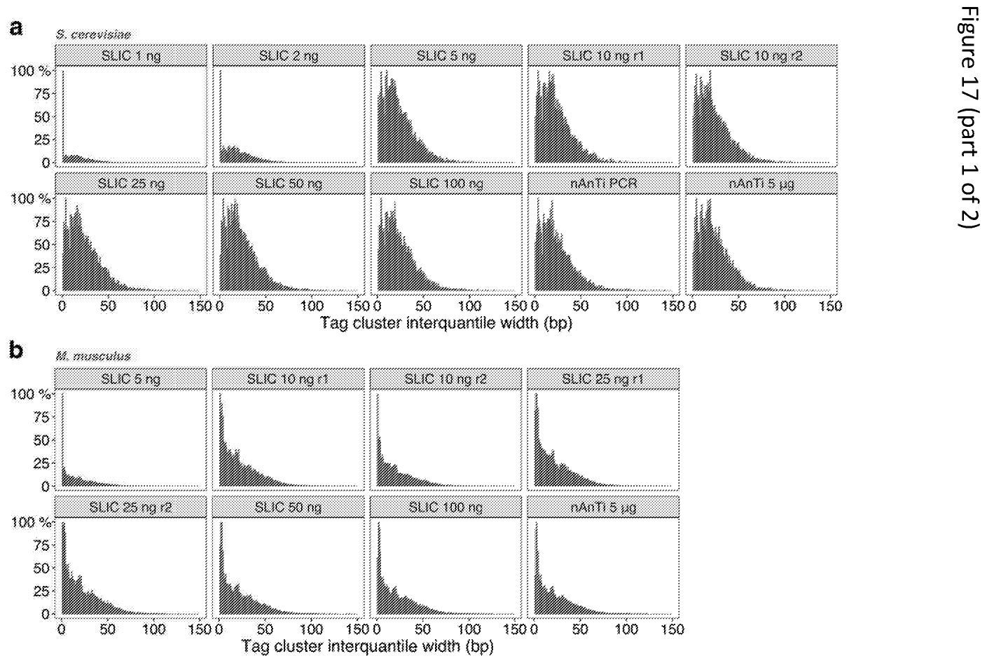

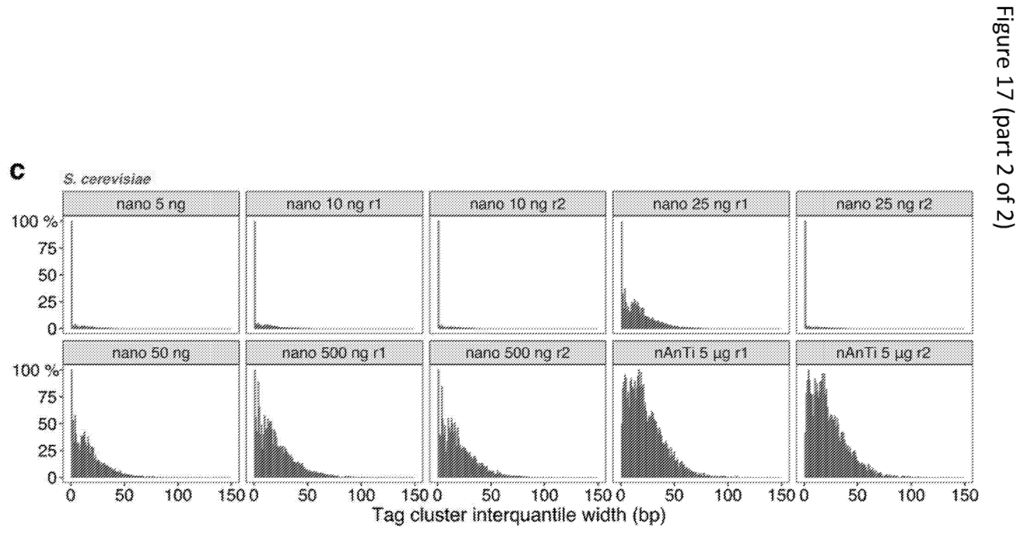

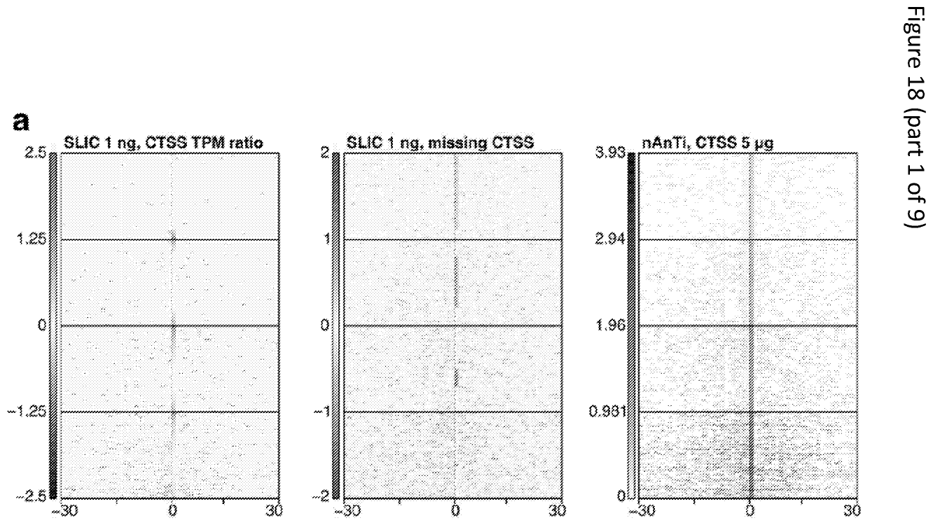

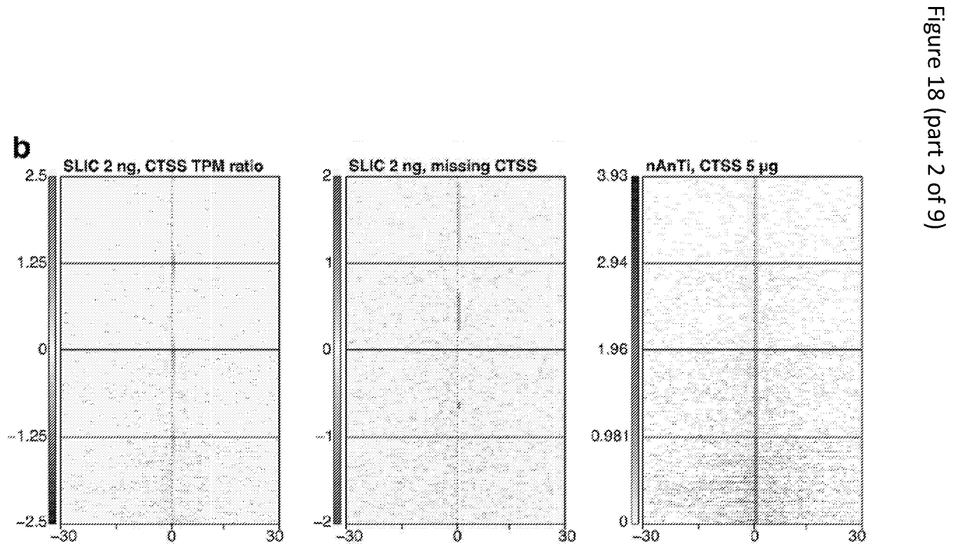

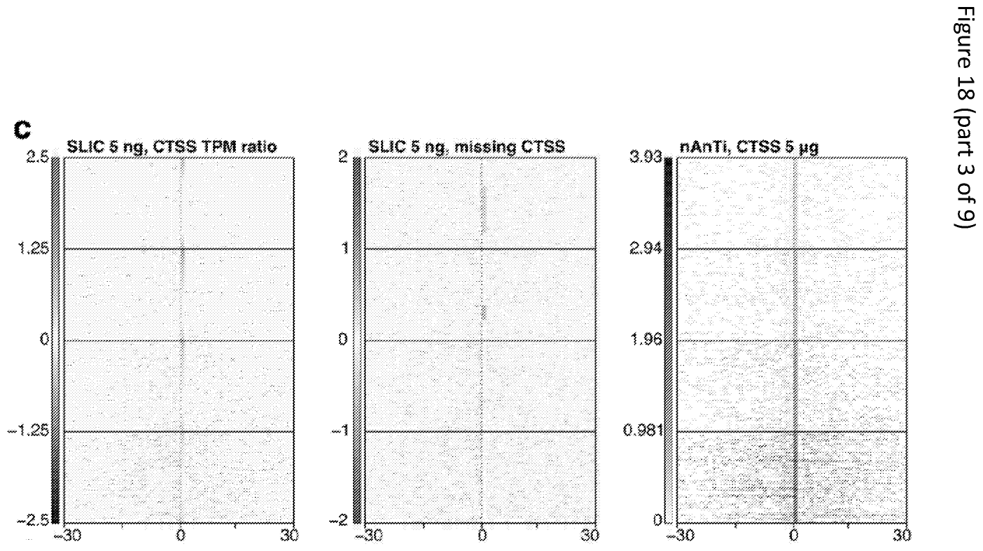

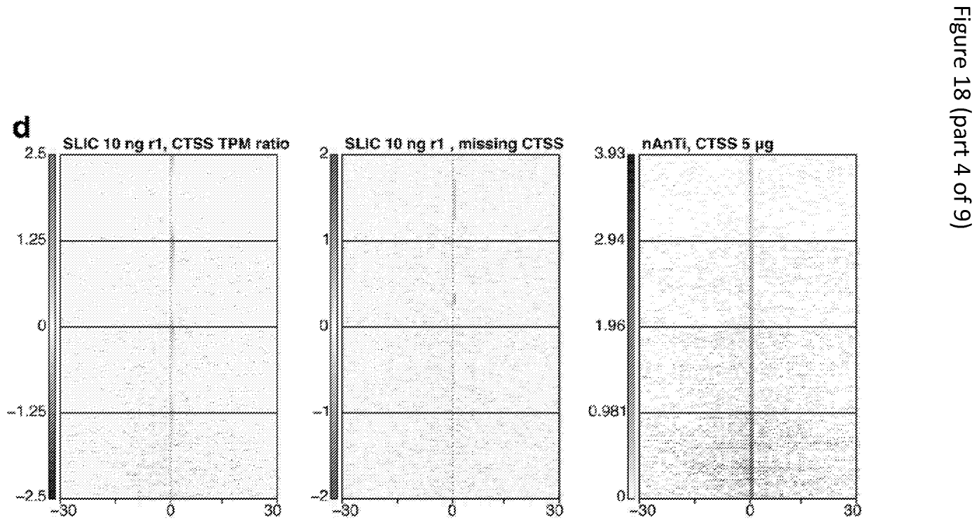

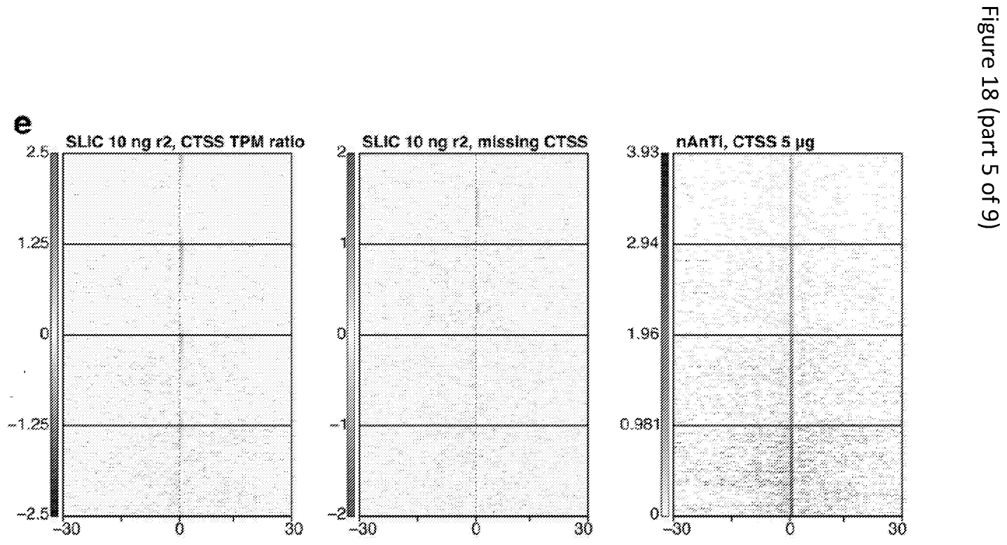

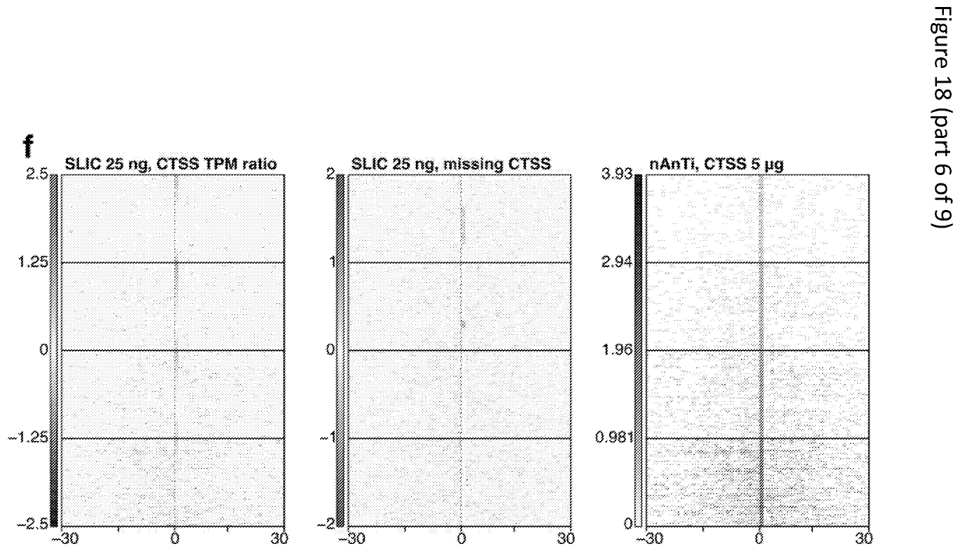

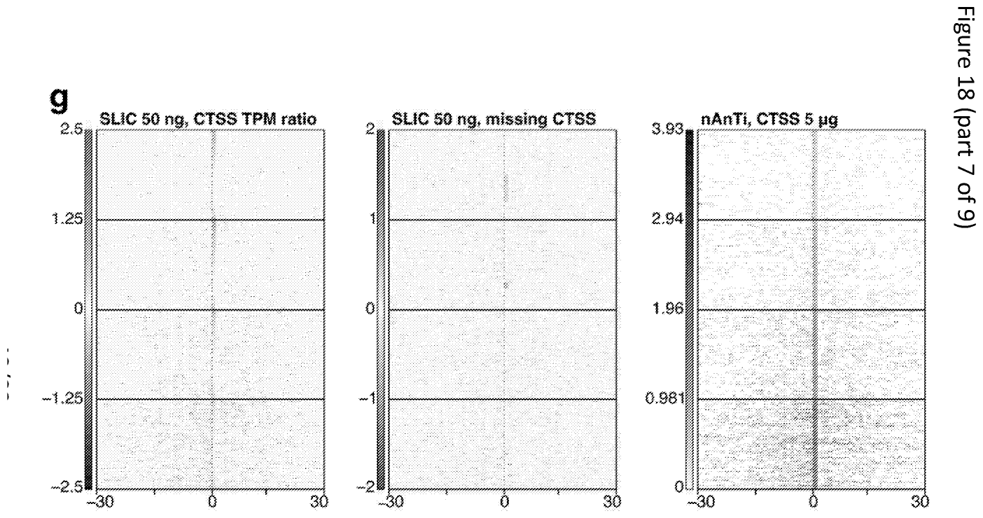

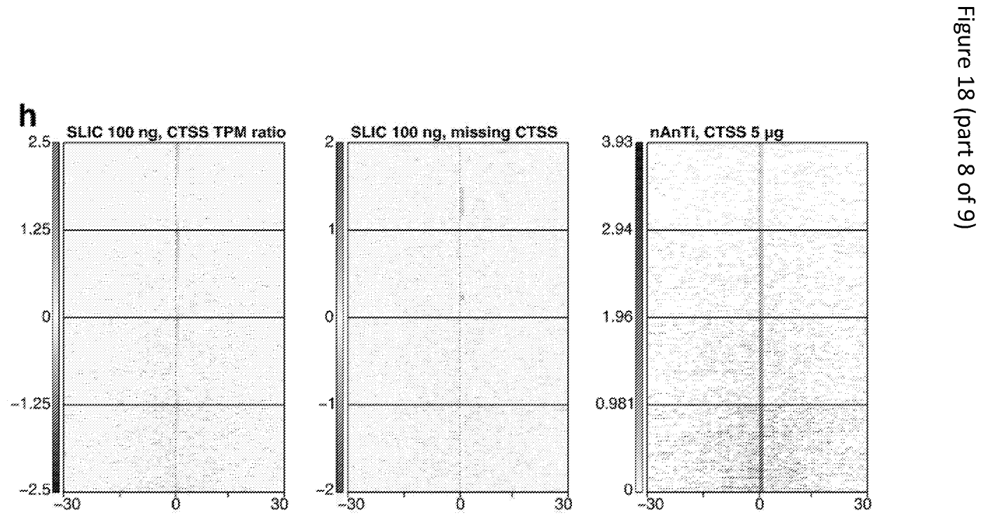

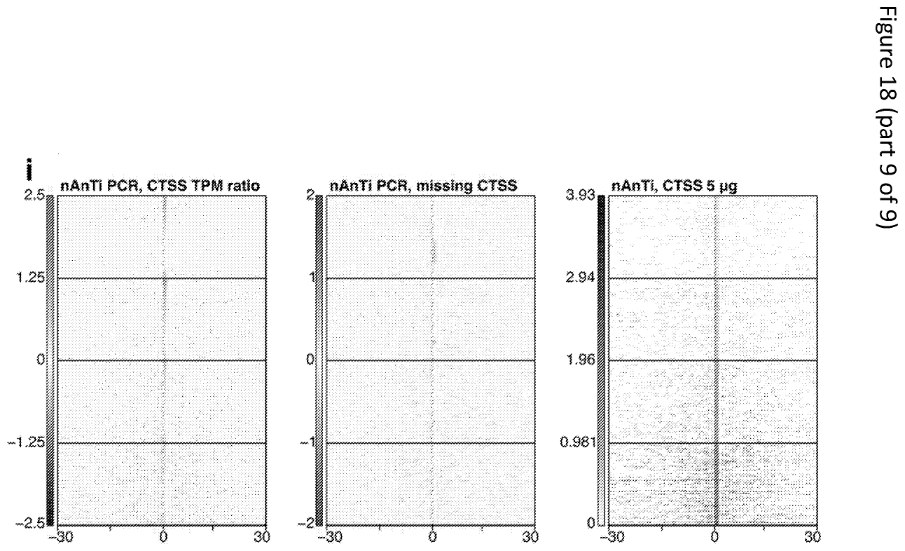

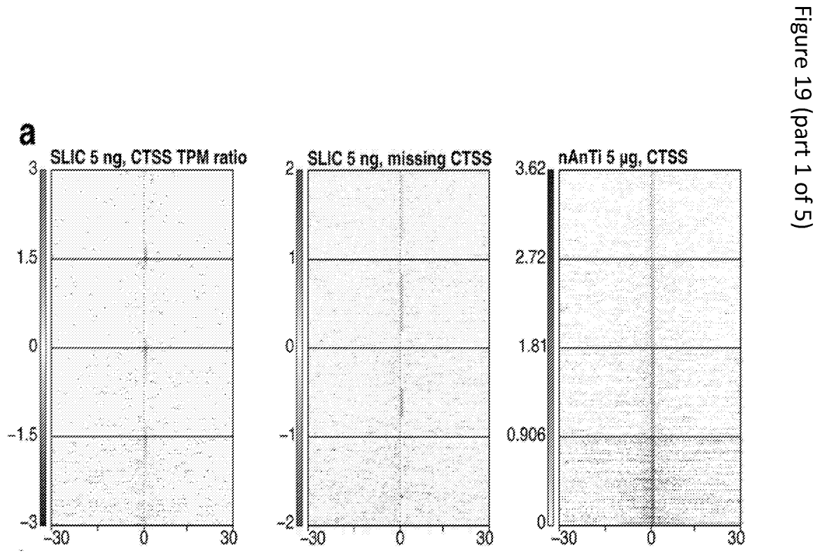

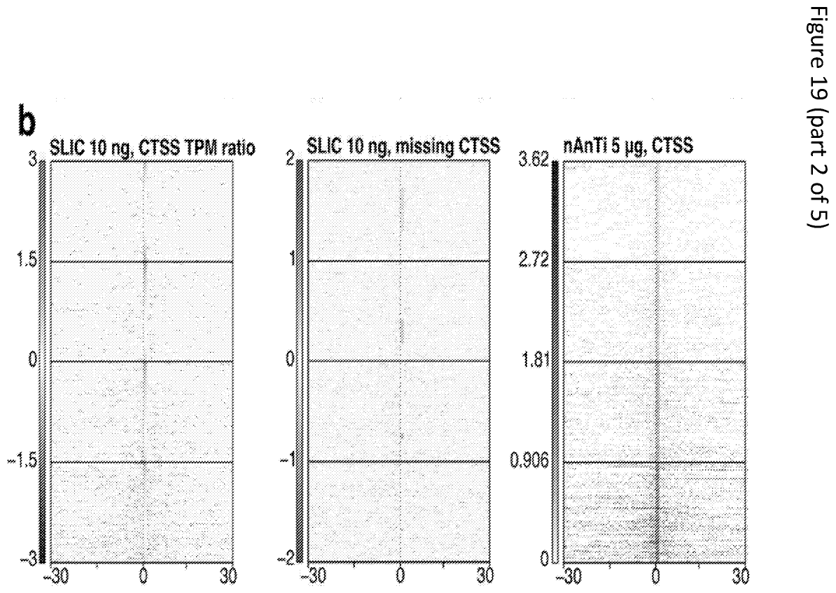

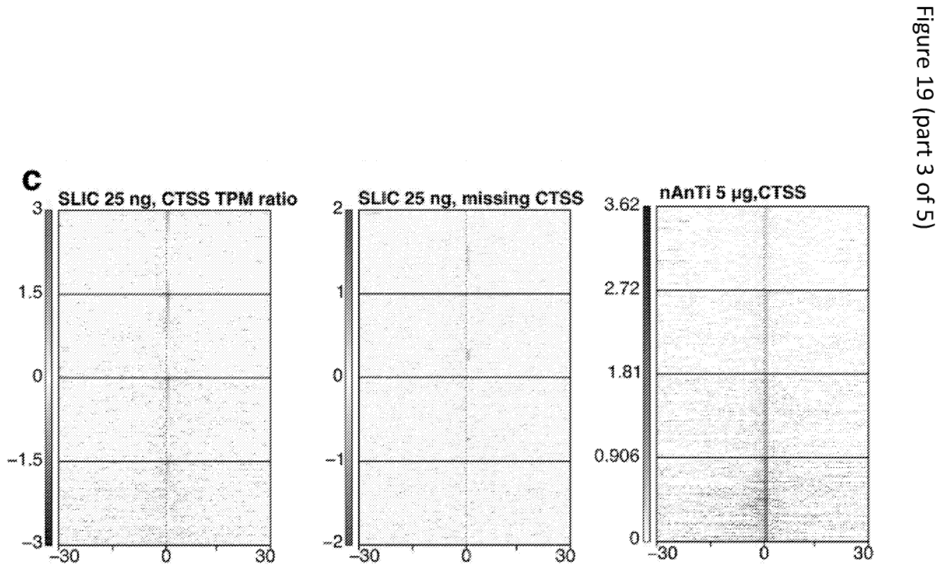

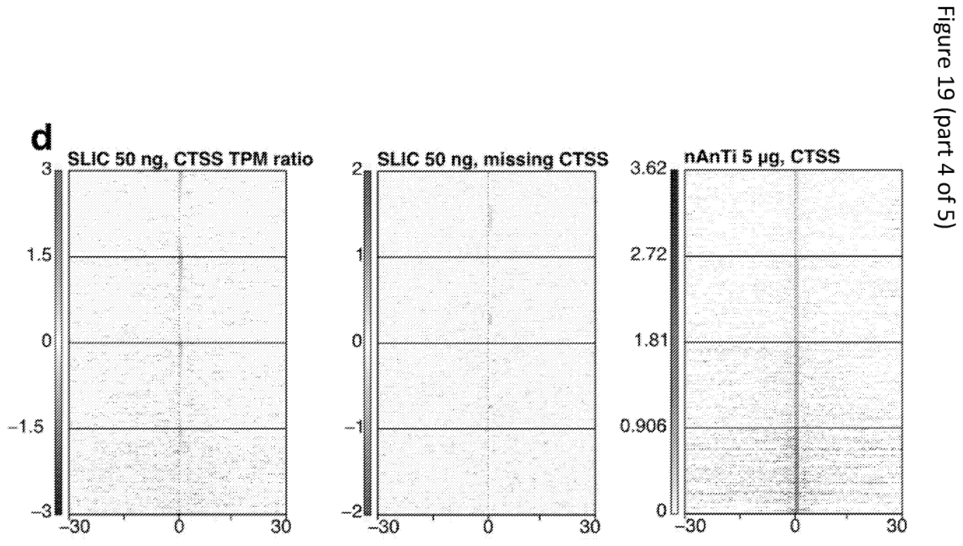

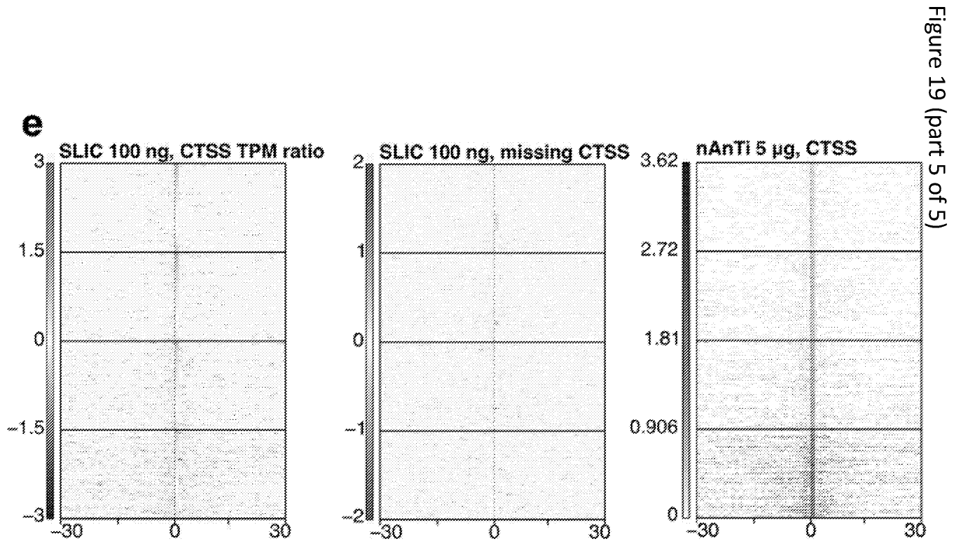

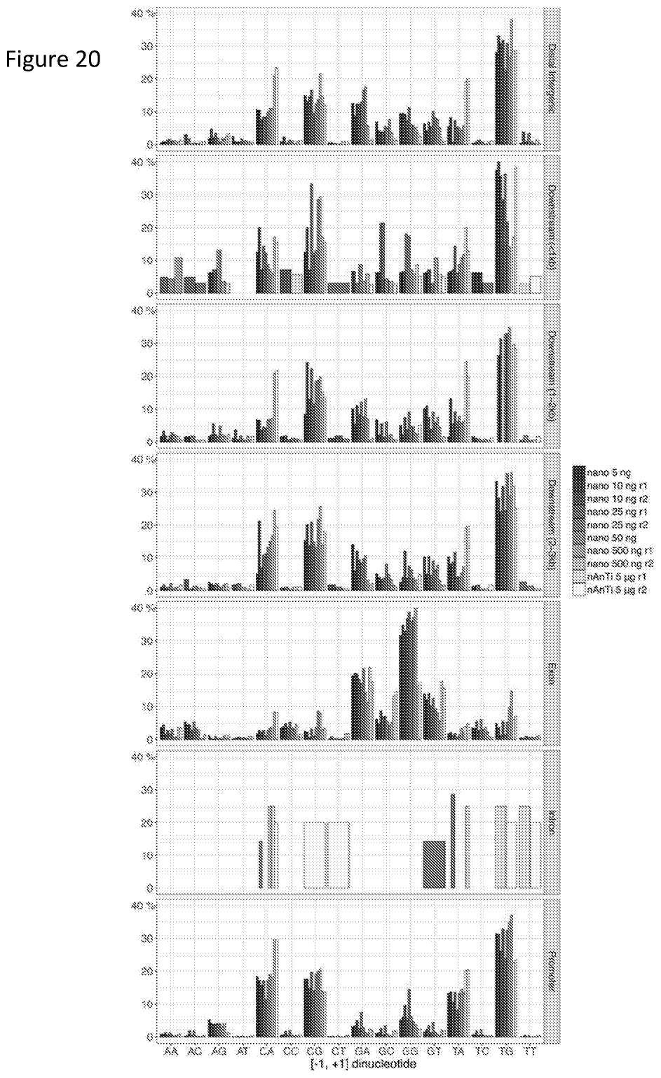

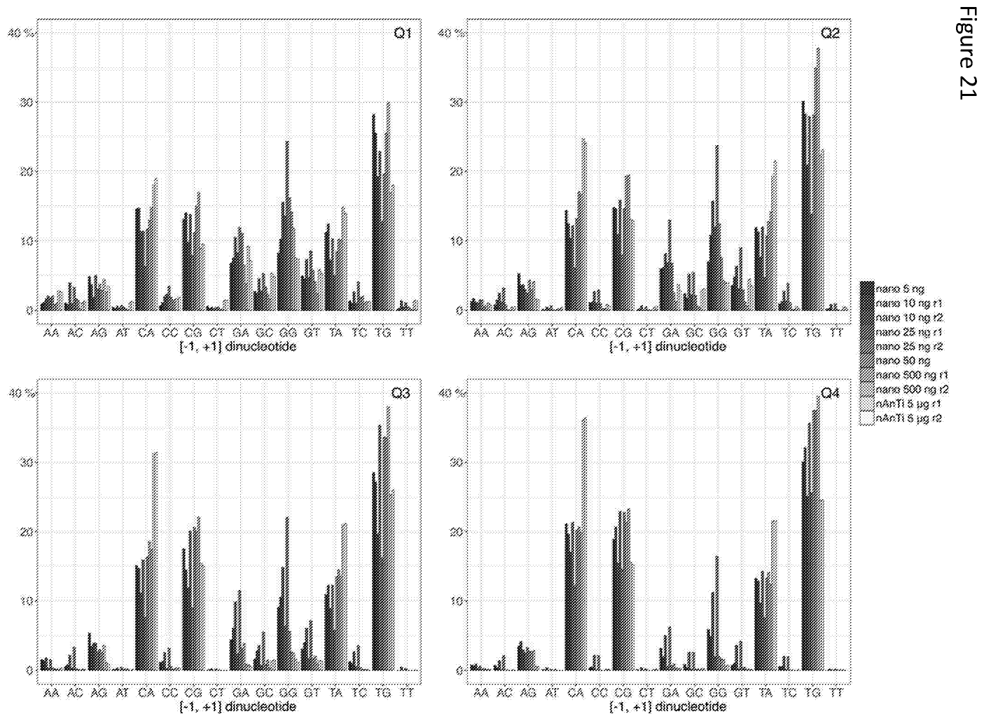

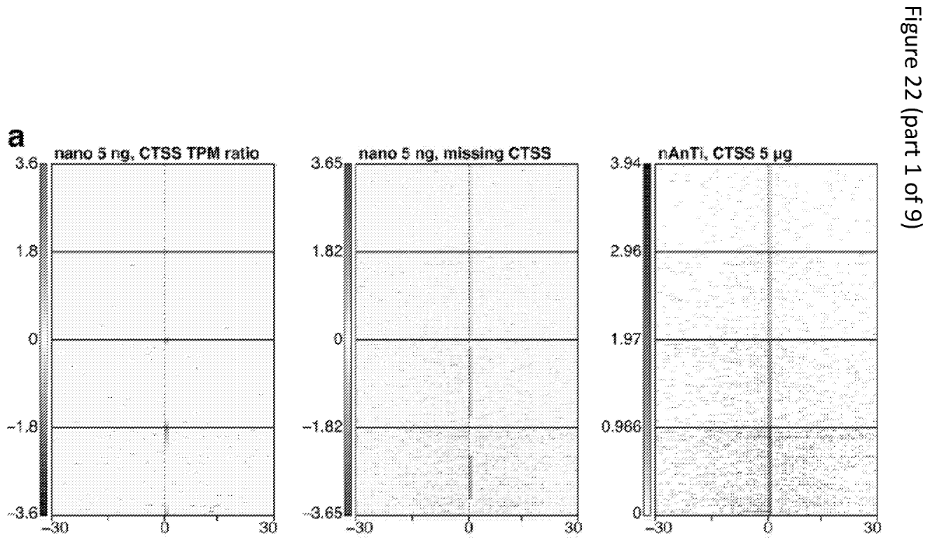

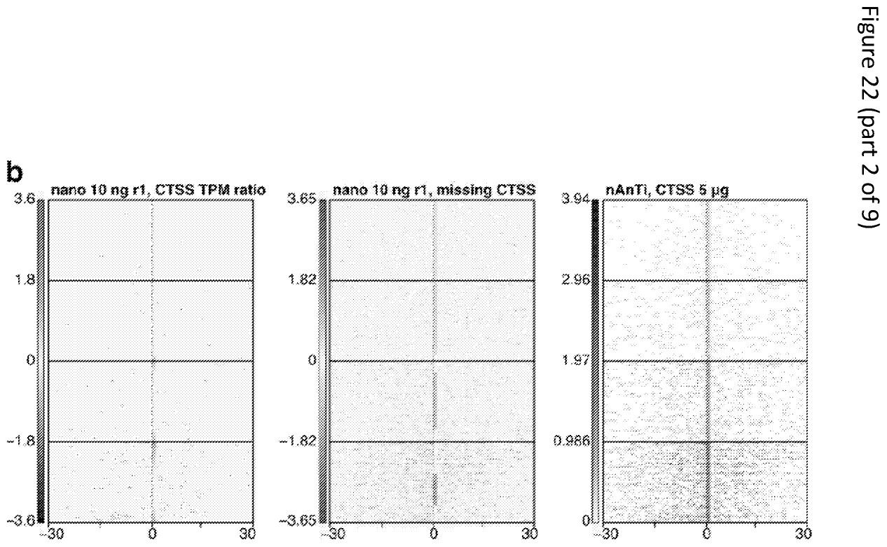

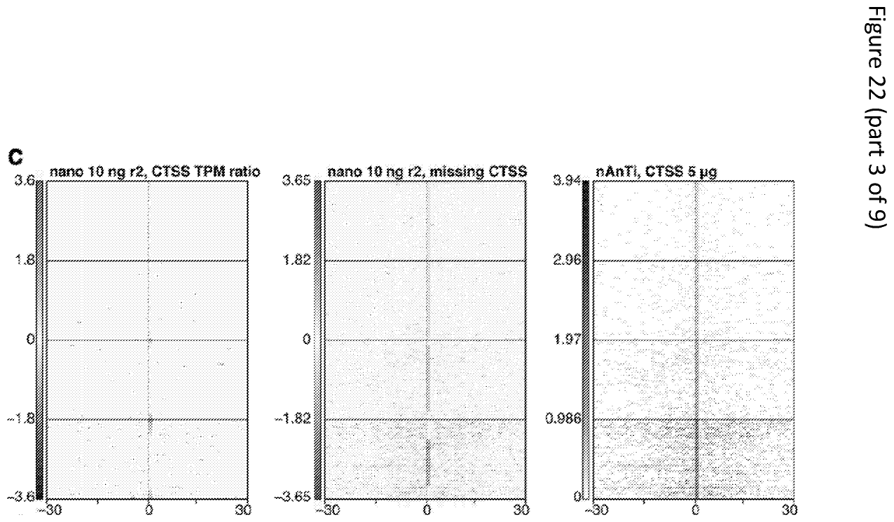

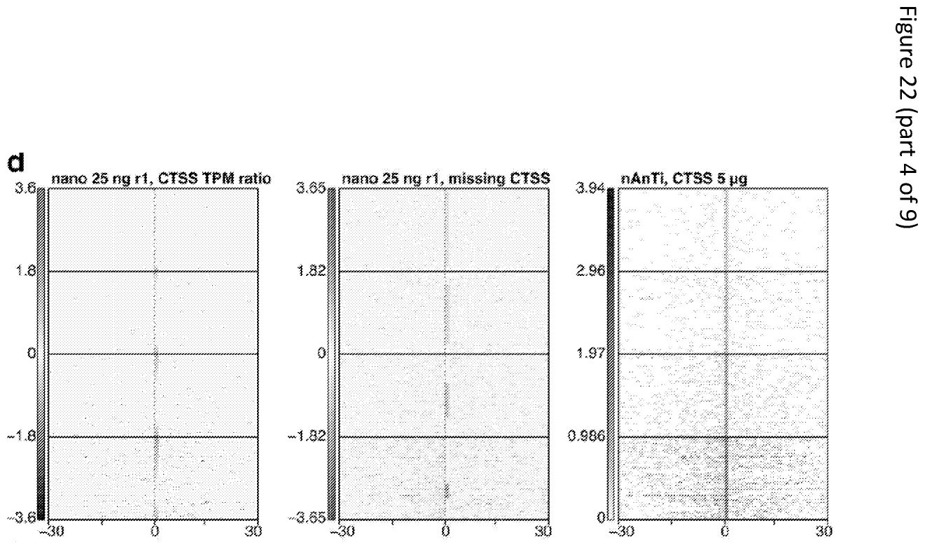

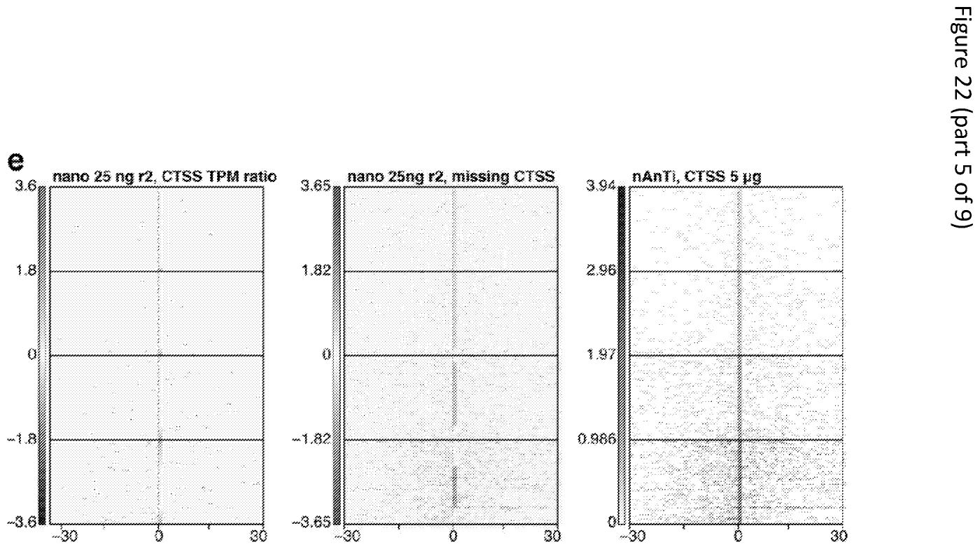

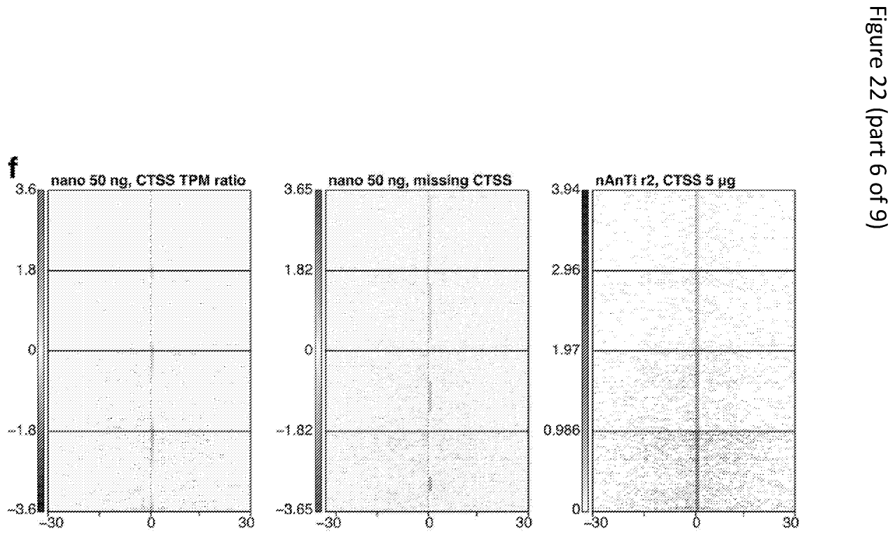

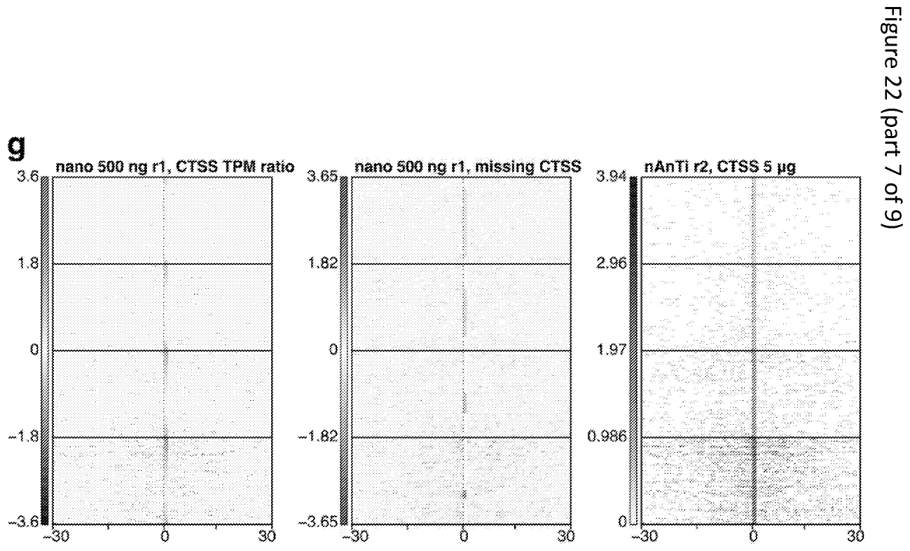

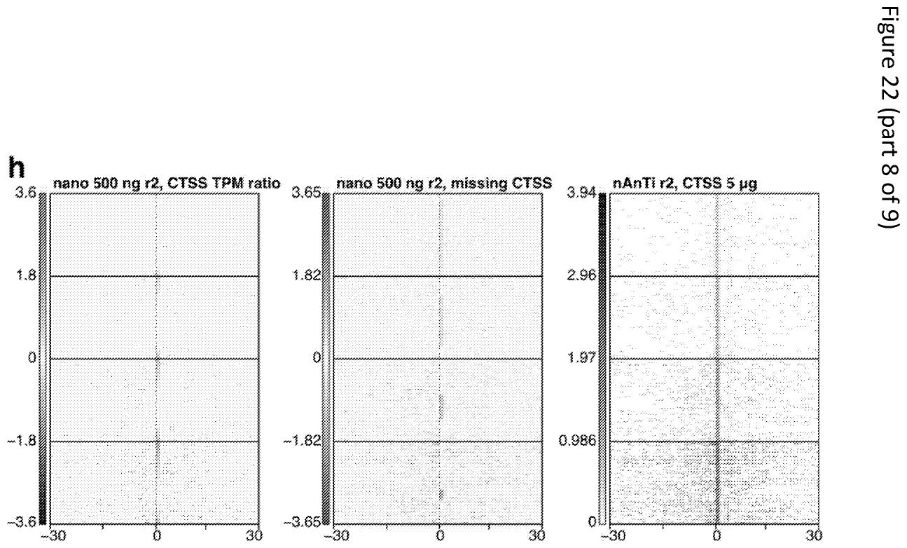

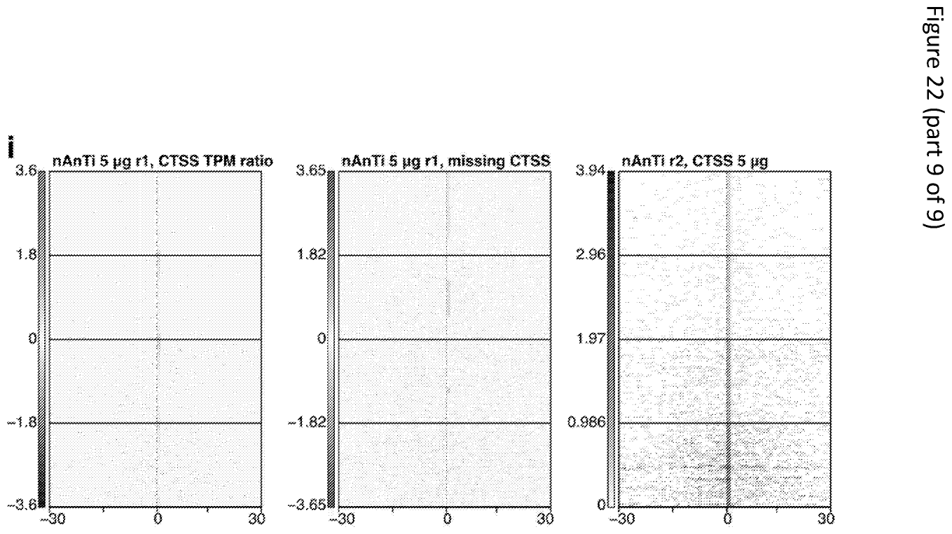

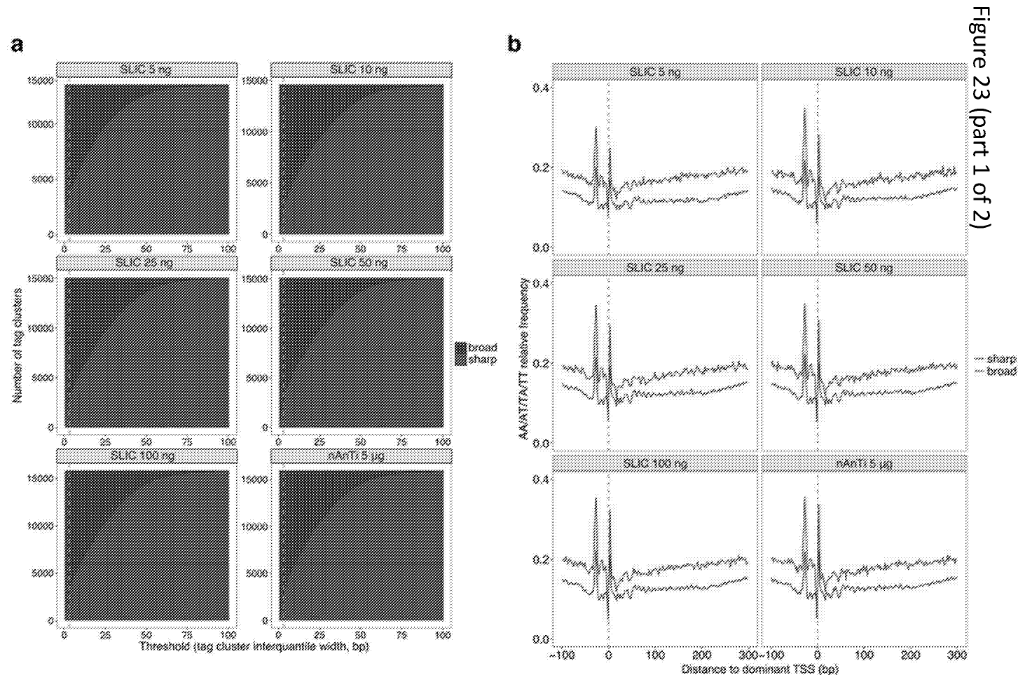

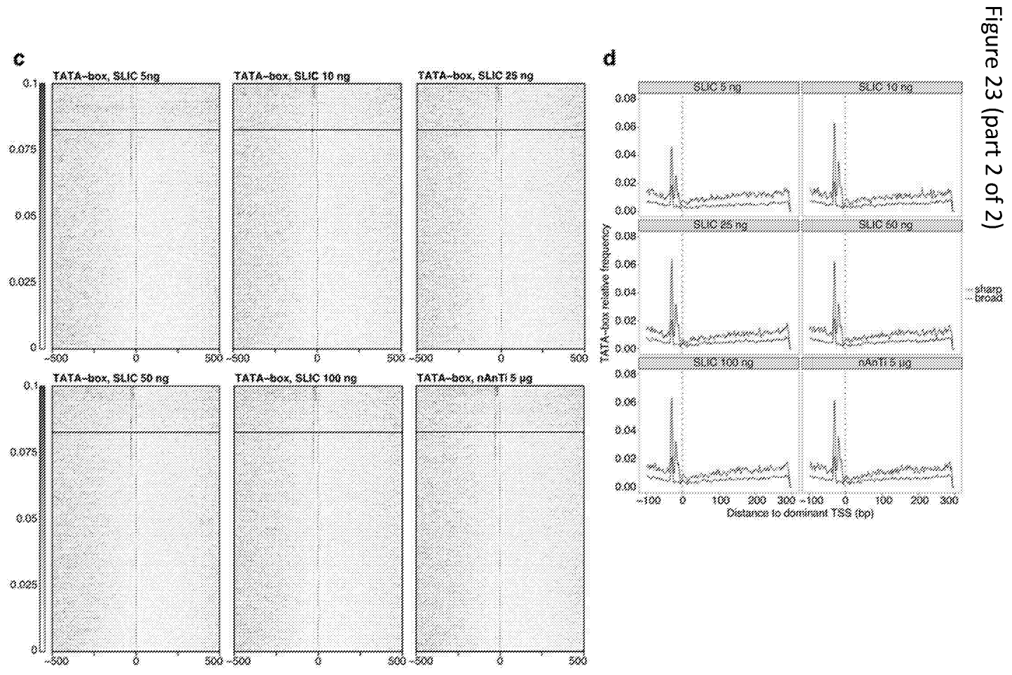

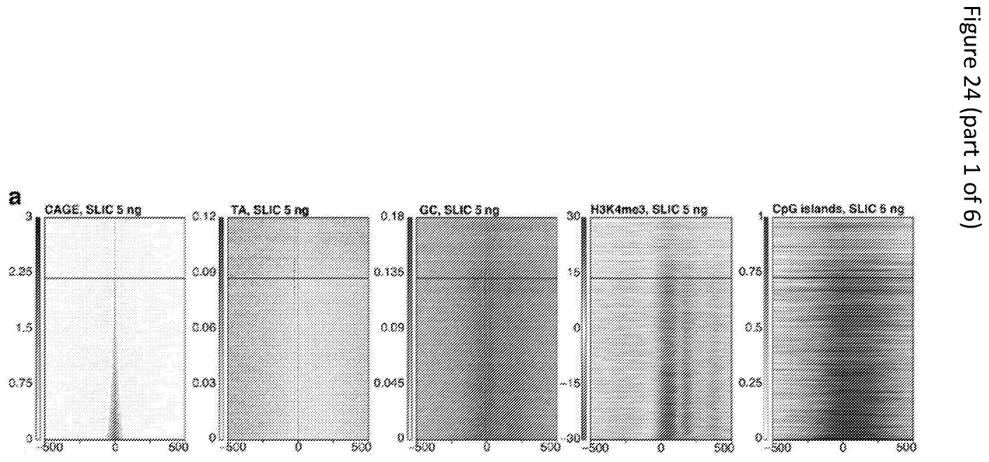

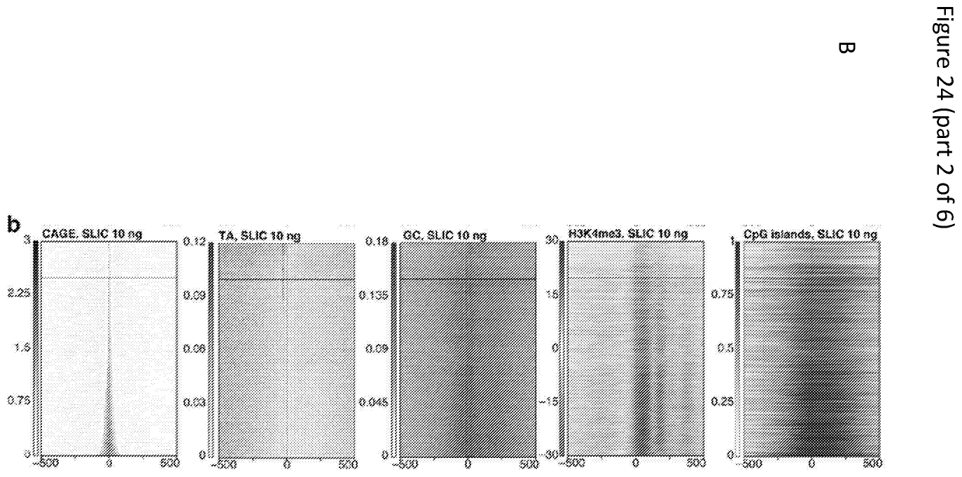

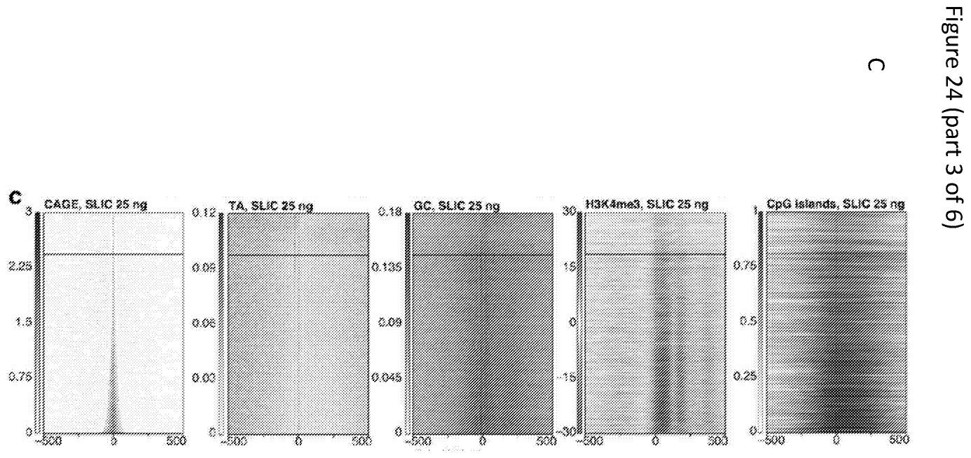

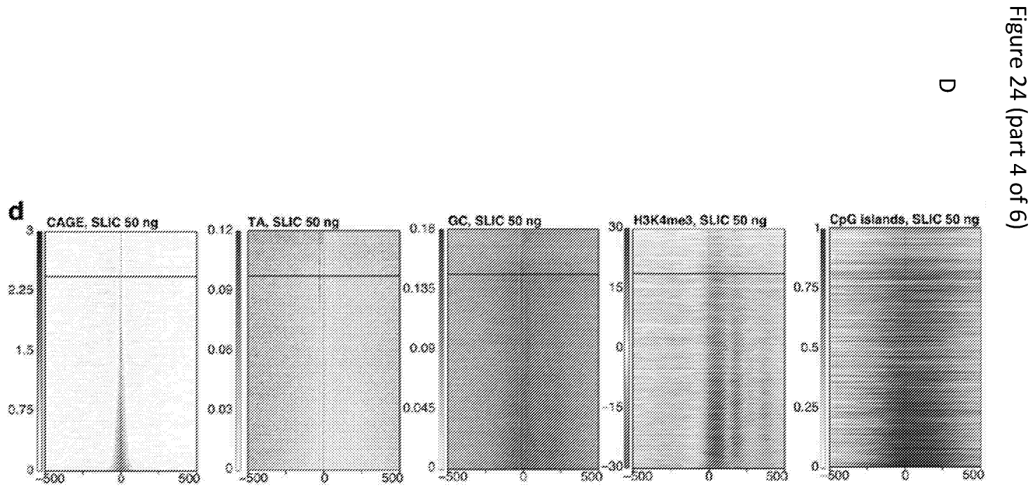

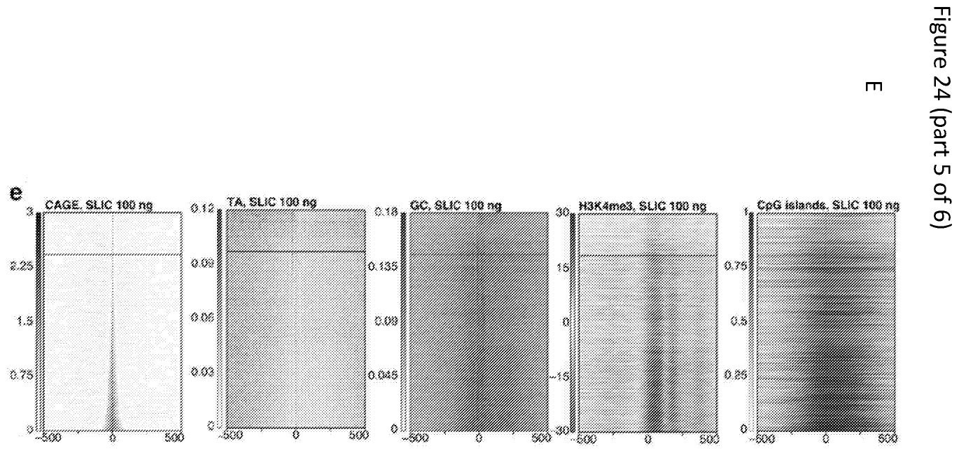

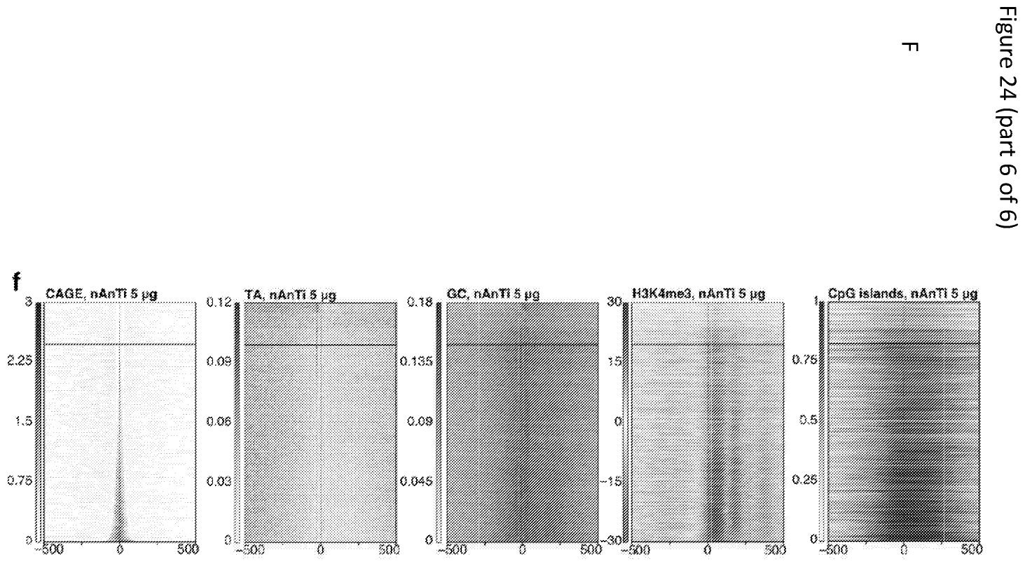

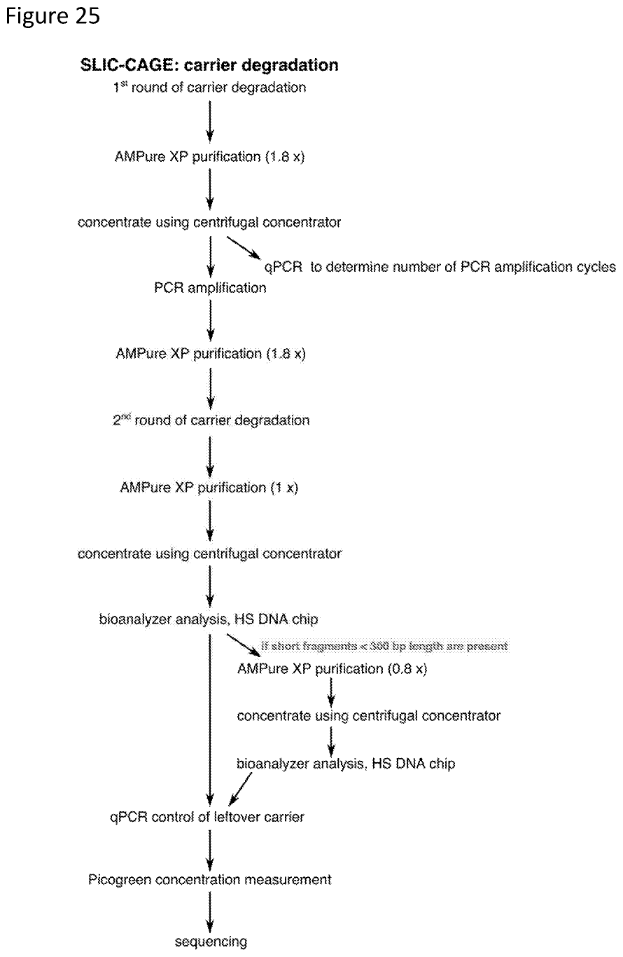

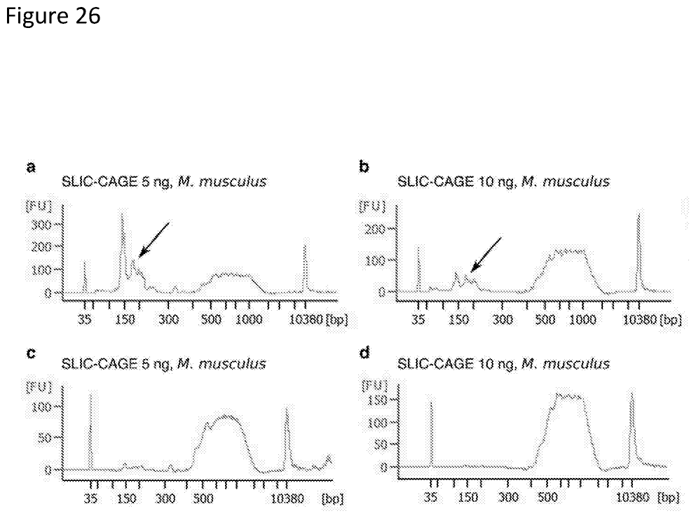

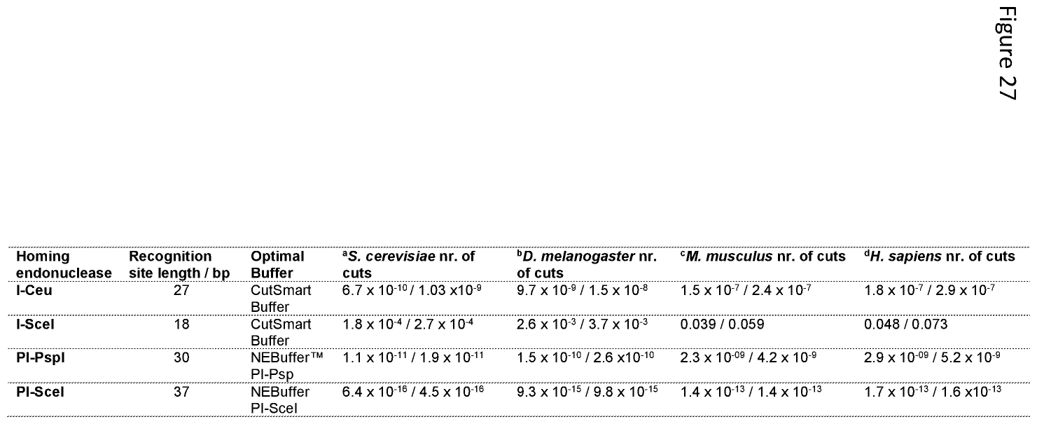

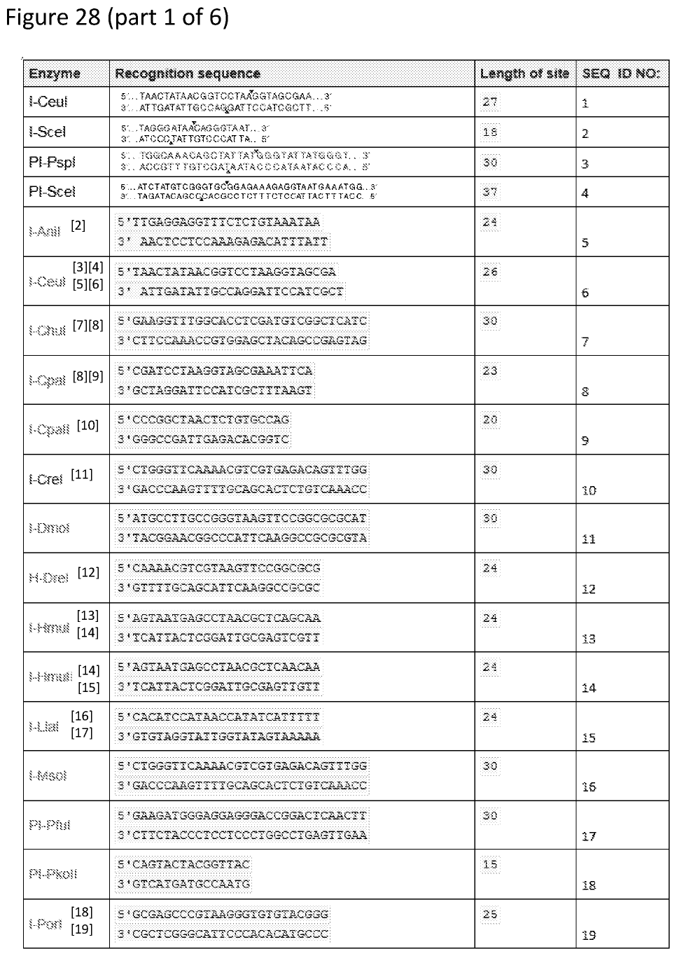

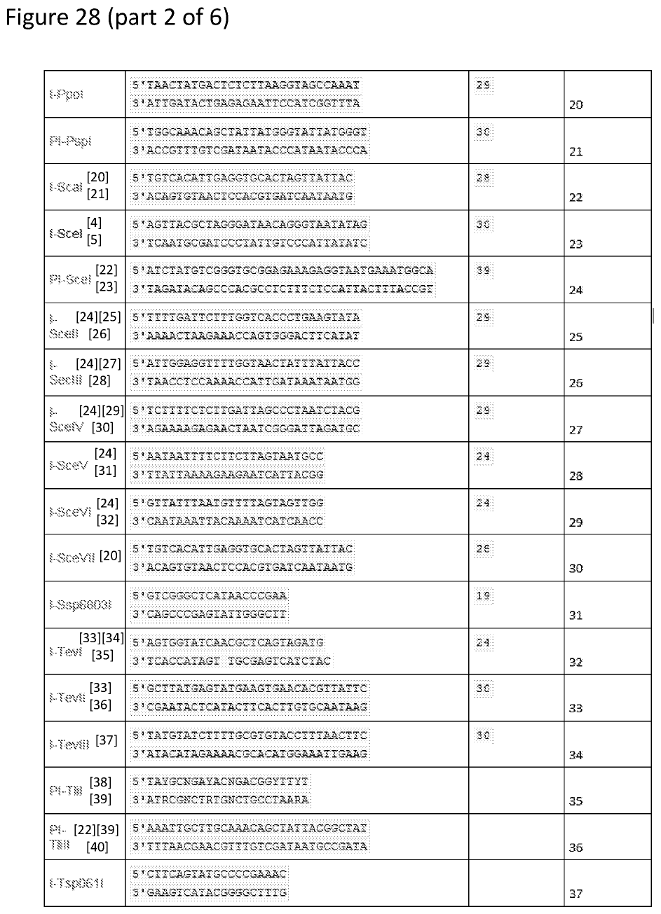

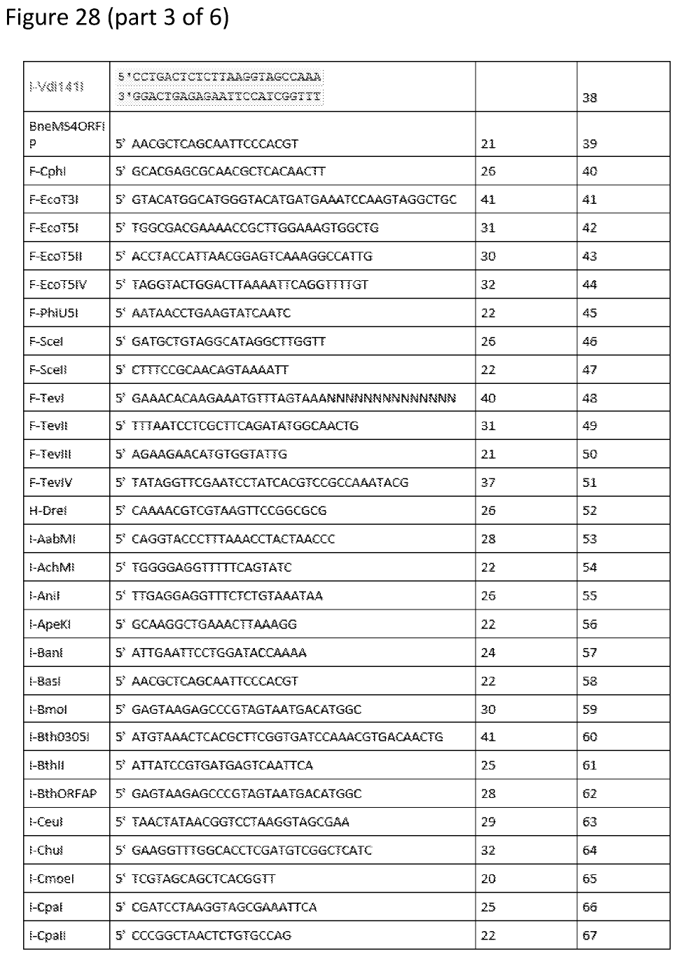

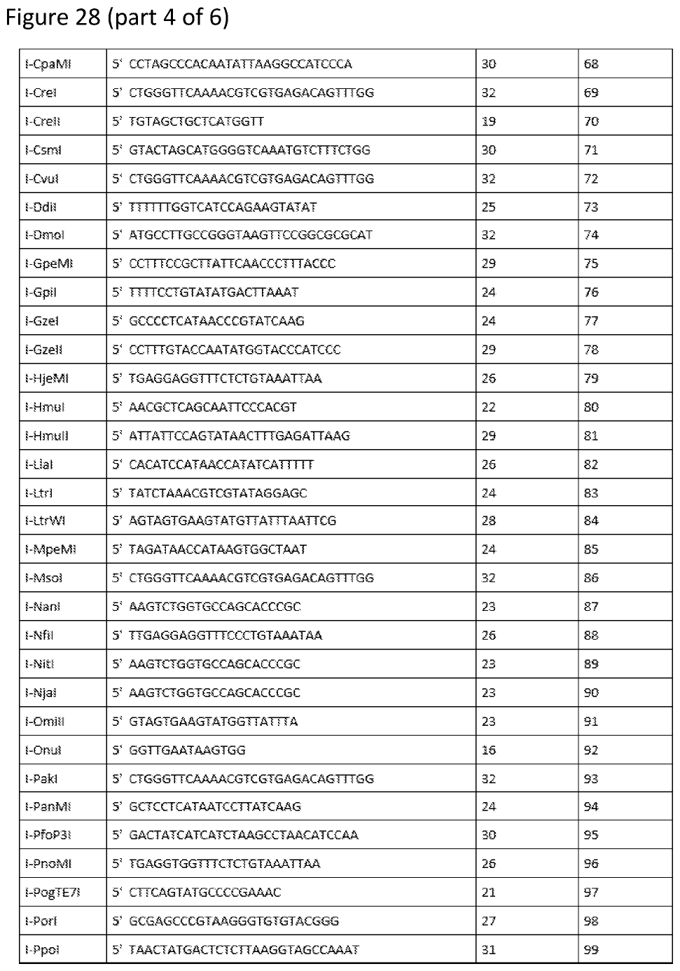

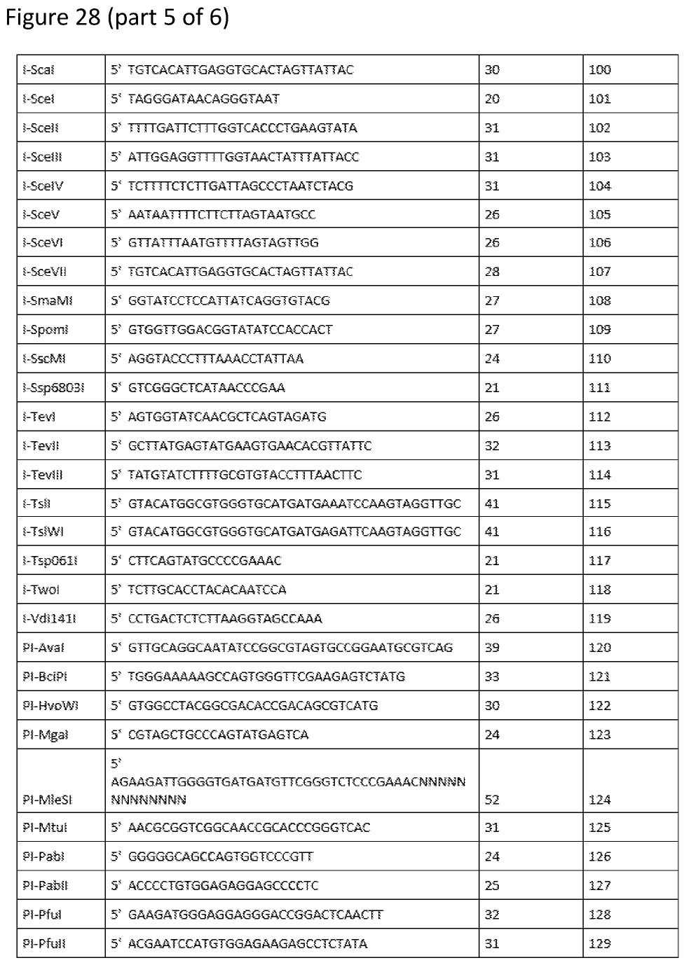

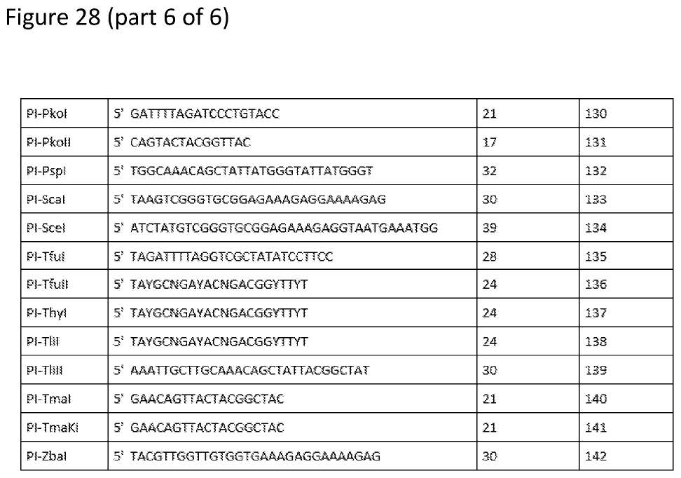

The inventors constructed the synthetic gene used as a template for run-off in vitro transcription of the carrier RNA (FIGS. 5 and 15 a,b). The synthetic gene is based on the Escherichia coli leucyl-tRNA synthetase sequence for two main reasons. The carrier nucleic acid should preferably not map to eukaryotic genomes. Secondly, leucyl-tRNA synthetase is a housekeeping gene from a mesophilic species and therefore its sequence is not expected to form strong secondary structures that would reduce its translation in vivo, or reduce the efficiency of reverse transcription to form RNA:cDNA hybrids. The carrier was made carrier selectively degradable by embedding it with multiple recognition sites of two homing endonucleases, I-CeuI and I-SceI (FIG. 1a, 5, 15a,b and 25). Combination of alternating recognitions sites allows for higher degradation efficiency and reduces sequence repetitiveness. The two enzymes have recognition sites of lengths 27 and 18 nucleotides, respectively, which even with some degeneracy allowed in the recognition site^{21, 22} makes their random occurrence in a transcriptome highly improbable. The two enzymes work at the same temperature and in the same buffer, so their digestion can be combined in a single step. A fraction of the synthesised carrier RNA is capped using Vaccinia Capping System (NEB) and mixed with uncapped carrier to achieve the desired capping percentage²³.

The percentage of capped RNAs in the carrier and its size distribution were optimised by performing the entire SLIC-CAGE protocol, starting by adding the synthetic carrier to the low-input sample to achieve a total of 5 μg of RNA material. To assess its performance, the output was compared with the nAnT-iCAGE library derived from 5 μg of total cellular RNA. nAnT-iCAGE was used as a reference as it is currently considered the most unbiased protocol for promoterome mapping¹⁴, and because TSS identification by cap-trapper based technology has been experimentally validated²⁴. To identify the optimal ratio of capped and uncapped carrier, as well as the length of the carrier RNAs, the following carrier mixes were tested: 1) carriers with lengths distributed between 0.3-1 kb versus homogenous 1 kb length carriers and 2) a mixture of capped and uncapped versus only capped carrier. The SLIC-CAGE protocol was performed as outlined in FIG. 1a, starting with 100 ng of total RNA isolated from S. cerevisiae supplemented with the various carrier mixes up to total 5 μg of RNA. We then compared the output with the nAnT-iCAGE library generated using 5 μg of total RNA (FIG. 7B, 8A, 15c-m). Removal of the carrier was performed by two rounds of degradation using homing endonucleases (I-SceI and I-CeuI, FIG. 25) with a purification and a PCR amplification step between the rounds. The presence of the carrier significantly improved the correlation of individual CAGE-supported TSSs (CTSSs) between SLIC-CAGE and the reference nAnT-iCAGE library (FIG. 15c-h). This effect was not observed when either only the capped carrier or no carrier was used (FIG. 15 c,g,h). The highest correlation and reproducibility was achieved by a carrier mix composed of 10% capped and 90% uncapped molecules of 0.3-1 kb length (mix 2, FIGS. 7B and 8A). This mix was designed to closely mimic the composition of cellular total RNA. Other diagnostic criteria shown in FIG. 15 j-m confirm this is the optimal carrier choice.

SLIC-CAGE Allows Genome-Wide TSS Identification from Nanogram-Scale Samples

To identify the lowest amount of total RNA that can be used to produce high quality CAGE libraries a SLIC-CAGE titration test was performed with 1-100 ng of total S. cerevisiae RNA (FIG. 16a) and compared with an nAnT-iCAGE library derived from 5 μg of total RNA. The high correlation of individual CTSSs between SLIC-CAGE and the reference nAnT-iCAGE library (FIGS. 1b and c, Supplementary FIG. 16a) shows that genuine CTSSs are identified. Moreover, SLIC-CAGE libraries show high reproducibility (FIG. 1d). FIG. 1e shows an example locus in the genome browser, demonstrating the high similarity of SLIC-CAGE and nAnT-iCAGE CTSS profiles in all high-quality datasets (i.e. datasets with high complexity, see below).

To confirm the general applicability of the SLIC-CAGE protocol, a similar titration test was performed using total RNA isolated from E14 mouse embryonic stem cells. The results obtained following sequencing of the libraries generated using 5, 10 or 25 ng of total RNA were highly correlated (Pearson correlation 0.9) with the reference nAnT-iCAGE derived library. The correlation did not improve further with increasing total starting RNA (FIG. 1f-h, 16c), again verifying the suitability of the SLIC-CAGE protocol for nanogram-scale samples. The genome browser view (FIG. 10 confirms the similarity of profiles on the individual CTSS level, although the library prepared from 5 ng of M. musculus total RNA exhibits minor differences, due to lower complexity as discussed in detail in the next section.

Analysis of library mapping efficiency demonstrated that selective degradation of the carrier is highly efficient. When only 1 ng of total RNA is used with a 5000-fold more carrier (5 μg), 25% of the sequenced reads are uniquely mapped to the target organism, while the rest corresponds to the leftover carrier (27%), short amplified linkers or multimappers, commonly discarded from TSS analyses (FIGS. 10 and 11). This amount of leftover carrier is minor and does not significantly compromise sequencing depth (10% or less when 10 ng of total RNA are used). It is expected that with additional rounds of degradation and purification, the leftover carrier could be further reduced, although with a risk of sample loss, and we found it unnecessary.

### Example 2—Complexity and Resolution of SLIC-CAGE Libraries

The complexity and any potential inherent CTSS detection biases of libraries produced using the SLIC-CAGE protocol was assessed. As discussed above, both SLIC-CAGE and nAnT-iCAGE libraries are highly correlated at individual CTSSs and so the spatial clustering of these CTSSs and its features was analysed.

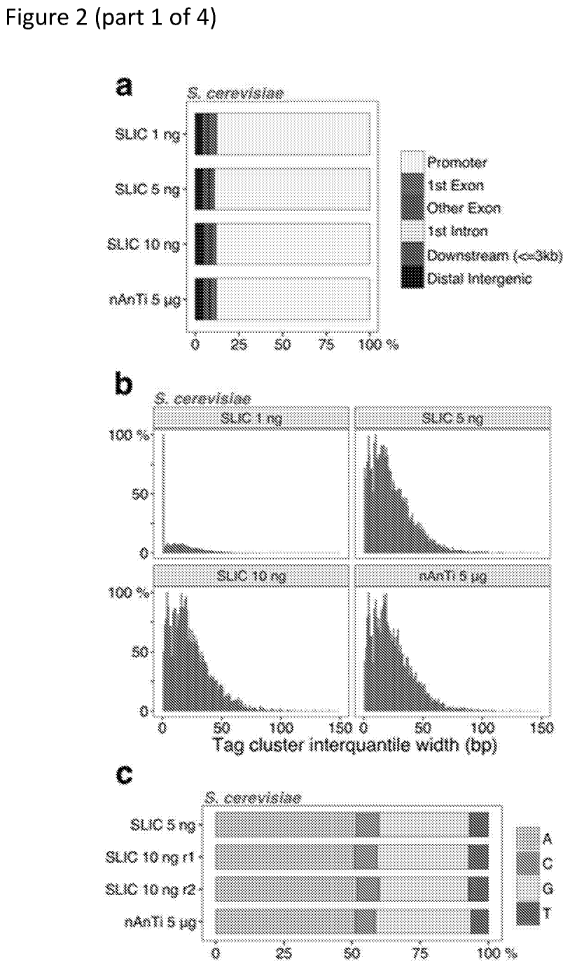

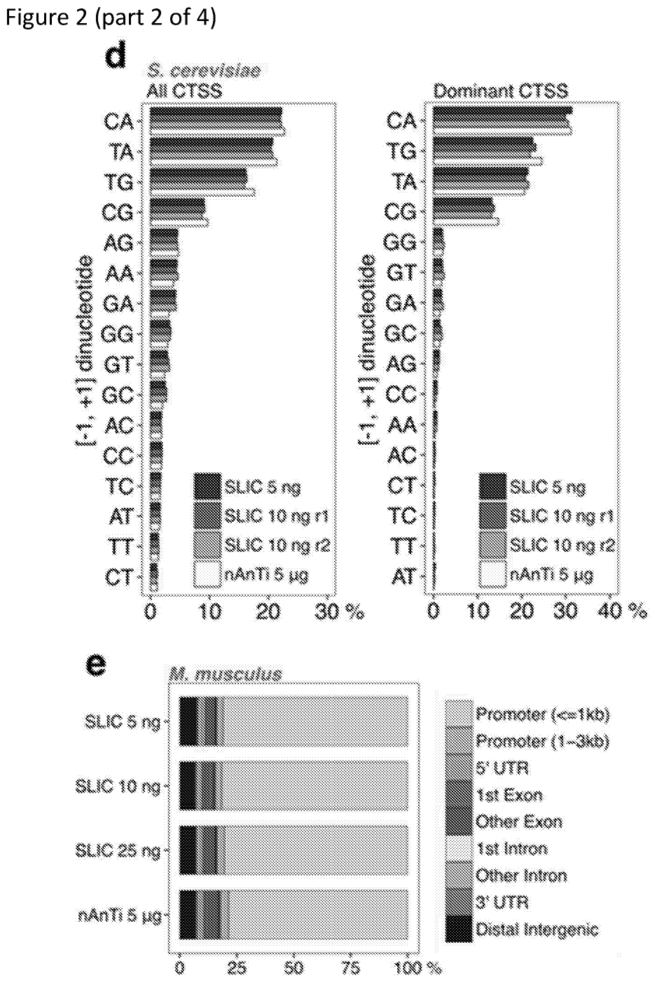

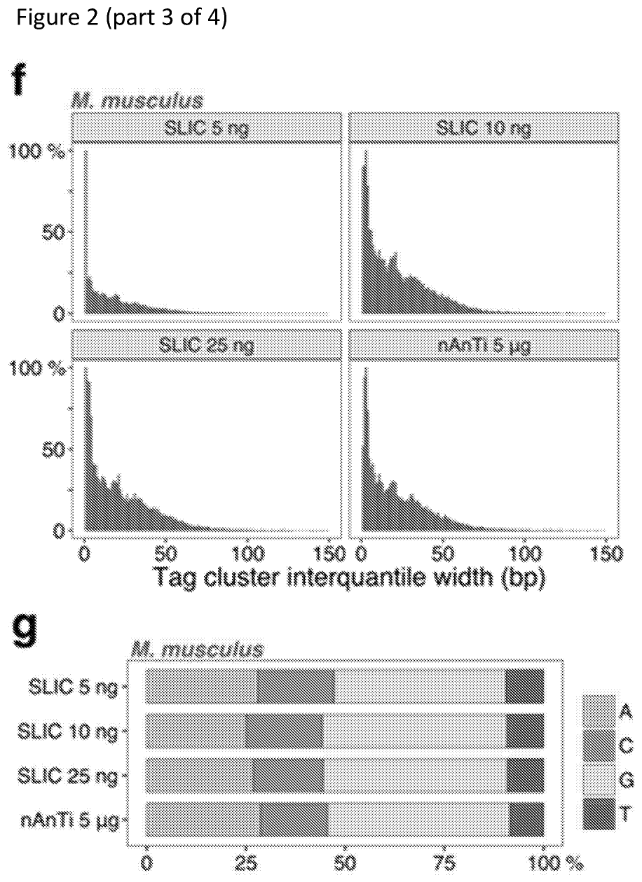

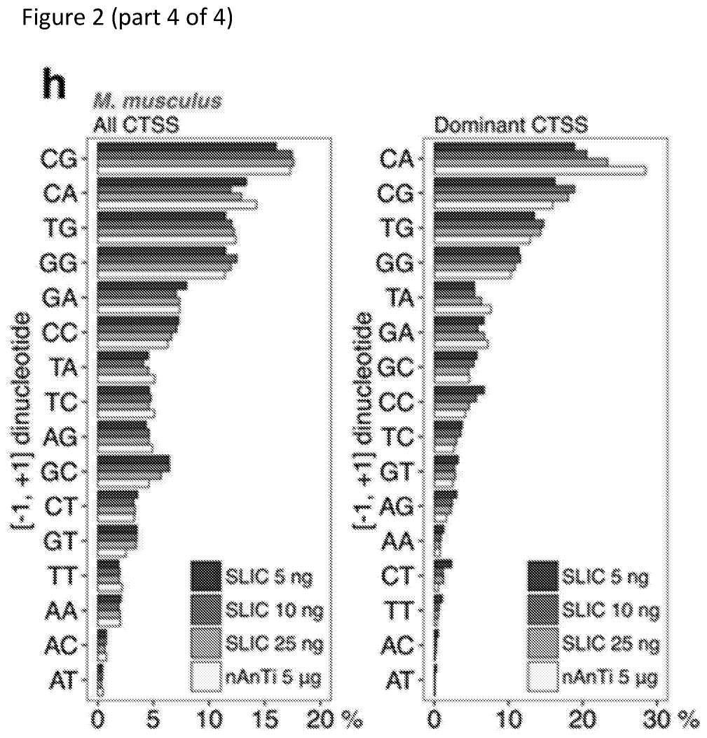

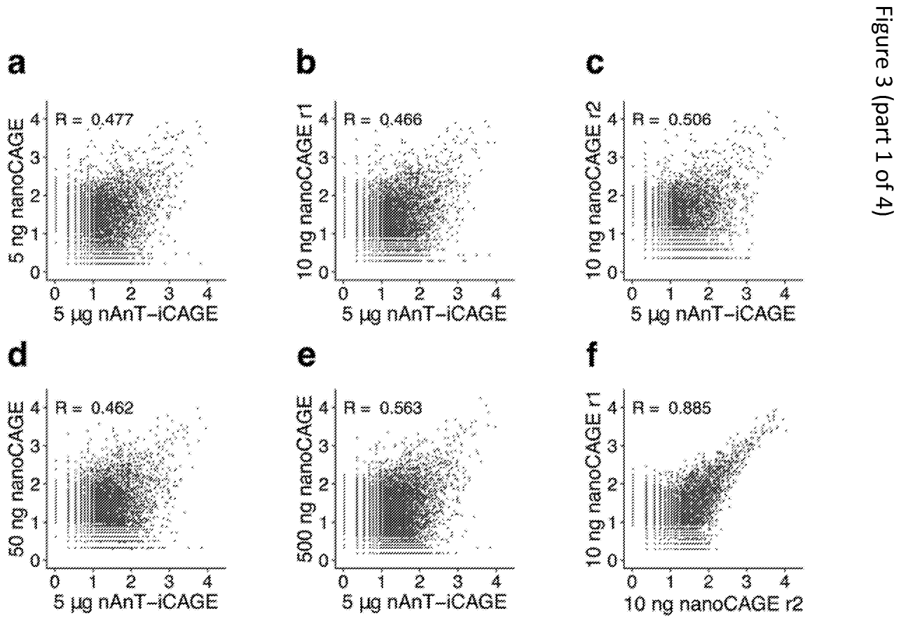

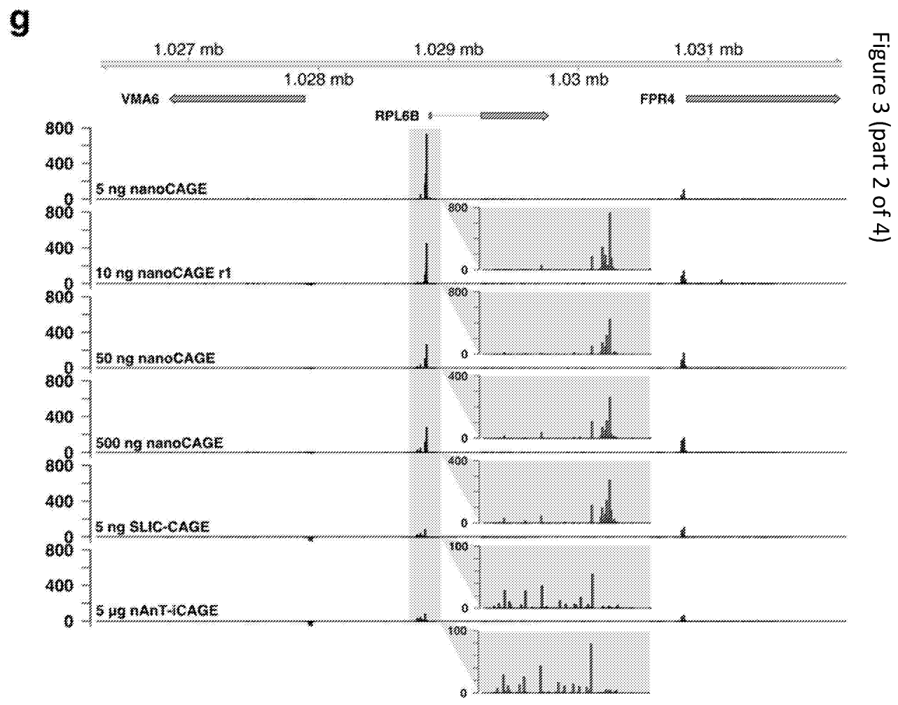

CTSSs in close vicinity reflect functionally equivalent transcripts and are generally clustered together and analysed as a single transcriptional unit termed a tag cluster²⁵. Specificity in capturing genuine TSSs can be assessed by examining the fraction of tag clusters that overlap with expected promoter regions. A high percentage of SLIC-CAGE tag clusters were identified that map to known promoter regions in both S. cerevisiae and M. musculus libraries irrespective of the total starting RNA, thus indicating the high specificity of these libraries (approximately 80%, at the same level as the reference nAnT-iCAGE protocol, FIG. 2a,e and FIG. 16d,f).

In addition to determining the number of unique CTSSs and tag clusters (FIG. 12), complexity of CAGE-derived libraries can be assessed by comparing tag cluster widths. To robustly identify tag cluster widths, the interquantile widths (IQ-width) were calculated that span 10th and the 90th percentile (q0.1-q0.9) of the total tag cluster signal to exclude effects of extreme outlier CTSSs. The distribution of tag cluster IQ-widths serves as a good indicator of library complexity. In low-complexity libraries, sparse CTSS detection will lead to artificially sharp tag clusters. IQ-width distribution of S. cerevisiae SLIC-CAGE tag clusters reveals that complexity of the reference nAnT-iCAGE library is recapitulated using as little as 5 ng of total RNA. This result is substantiated with the number of unique CTSSs which corresponds to the number identified with nAnT-iCAGE (around 70% overlap between 5 ng SLIC-CAGE and nAnTi-iCAGE, and 90% overlap in tag cluster identification). Low-complexity with artificially sharper tag clusters is seen only with 1-2 ng of total RNA input (FIGS. 2b, 12 and 17a). A highly similar result is observed with M. musculus SLIC-CAGE libraries, although lower complexity is notable at 5 ng of total RNA (FIGS. 2f and 17b). This is in agreement with the lower number of unique CTSSs identified in 5 ng M. musculus SLIC-CAGE library compared to nAnT-iCAGE (FIG. 12). It is expected that an increase in sequencing depth would ultimately recapitulate the complexity of the reference dataset as higher coverage in S. cerevisiae facilitates higher complexity libraries with lower starting amount (5 ng).

SLIC-CAGE derived CTSS features from S. cerevisiae and M. musculus were also assessed and compared them with features extracted using the nAnT-iCAGE library as reference. First, nucleotide composition of all SLIC-CAGE-identified CTSSs reveals highly similar results to nAnT-iCAGE independent of the total input RNA (FIGS. 2c,g and 16g,i). Furthermore, the composition of [−1,+1] dinucleotide initiators (where the +1 nucleotide represents the identified CTSS) also showed a highly similar pattern to the reference nAnT-iCAGE dataset (FIG. 2d,h left panel, and 16j,i). SLIC-CAGE libraries identify CA, TA, TG and CG as the most preferred initiators, similar to preferred mammalian initiator sequences²⁴.

Focusing only on the initiation patterns ([−1, +1] dinucleotide) of the dominant TSS (CTSSs with the highest TPM within each the tag cluster) of each tag cluster facilitates estimation of the influence of PCR amplification on the distribution of tags within a tag cluster. Highly similar dinucleotide composition of dominant TSS initiators, independent of the amount of total RNA used, confirms that identification of the dominant TSSs is not obscured by PCR amplification (FIG. 2d,h right panel, and 16m,o). The identified preferred initiators are pyrimidine-purine dinucleotides CA, TG, TA (S. cerevisiae) or CA, CG, TG (M. musculus) in accordance with the lnr element (YR) 7, 26. These results confirmed the utility of SLIC-CAGE in uncovering authentic transcription initiation patterns such as the well-established CA initiator.

As a final assessment of SLIC-CAGE performance, the expression ratios per individual CTSS common to SLIC-CAGE and the reference nAnT-iCAGE were analysed (FIGS. 18 and 19 left panels) and present the ratios in a heatmap centred on the dominant CTSS identified by the reference nAnT-iCAGE library. This analysis can uncover any positional biases, if introduced by the SLIC-CAGE protocol. Patterns of signal in heatmaps (grouping upstream or downstream of the nAnTi-iCAGE-identified dominant CTSS) would signify positional bias and indicate non-random capturing of authentic TSSs. The positions and expression values of CTSSs identified in the nAnT-iCAGE but absent in SLIC-CAGE libraries were also evaluated (FIGS. 18 and 19, middle panels). No positional biases with regards to SLIC-CAGE-identified CTSSs and their expression values were identified, independent of the total input RNA. As expected, a higher number of CTSSs identified in nAnT-iCAGE were absent from lower complexity S. cerevisiae SLIC-CAGE libraries derived from 1 and 2 ng total RNA (FIGS. 18a and b, middle panels). This was particularly evident in those CTSSs with expression values in the lower two quartiles (top two sections in each heatmap). Further, the CTSSs identified in both low-complexity SLIC-CAGE and nAnT-iCAGE exhibit higher TPM ratios, likely reflecting the effect of PCR amplification. On the other hand, the SLIC-CAGE library derived from 5 ng of total RNA (FIG. 18c) shows similar patterns as libraries derived from greater amounts of RNA (FIG. 18d-h) or the library derived by PCR amplification of the nAnT-iCAGE library (FIG. 18i).

Similar results were obtained when comparing M. musculus SLIC-CAGE libraries with their reference nAnT-iCAGE library (FIG. 19), albeit with a twofold greater minimum starting RNA (10 ng) required for high-complexity libraries. Overall, these results show that SLIC-CAGE increases the sensitivity of the CAGE method 1000-fold over the current “gold standard” nAnT-iCAGE, without decrease in signal quality. This unparalleled sensitivity positions SLIC-CAGE as a method of choice for unbiased identification of TSSs in low-input samples that were previously inaccessible to CAGE methodology.

### Example 3—SLIC-CAGE Generates Superior Quality Libraries Compared to Existing Low Input Methods

The current available method for low input samples, nanoCAGE, requires 50-500 ng of total cellular RNA and is very different from standard CAGE in its selection of capped RNAs^{15, 16}. Whilst the gold standard verified CAGE protocols, i.e. nAnT-iCAGE relies on cap-trapper based selection of capped RNA, nanoCAGE uses the template switching property of the reverse transcriptase to selectively introduce a barcoded adapter only onto 5′ ends of capped RNA. The result are hybrid cDNA molecules with a specific nucleotide sequence added to the 5′ end of the capped RNA.

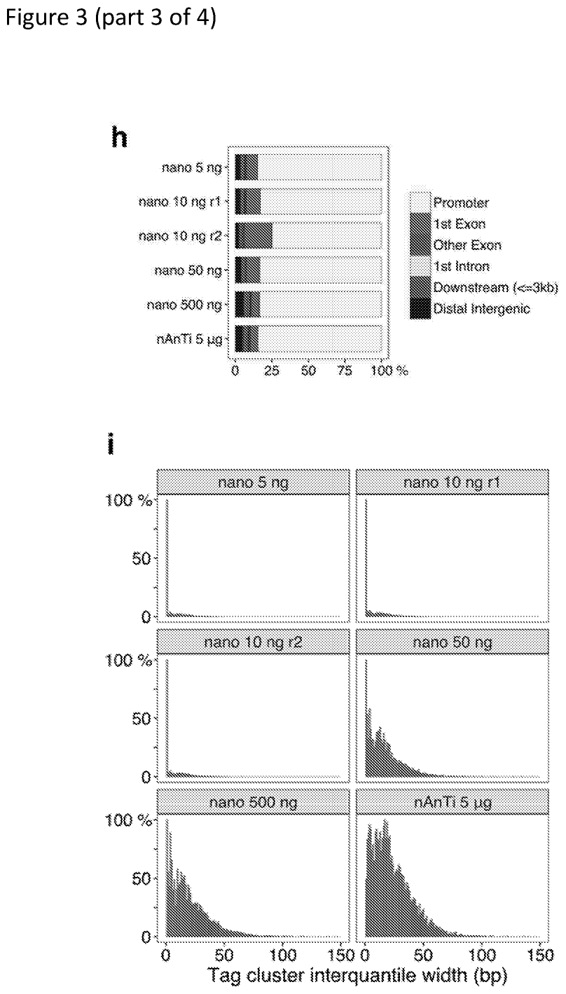

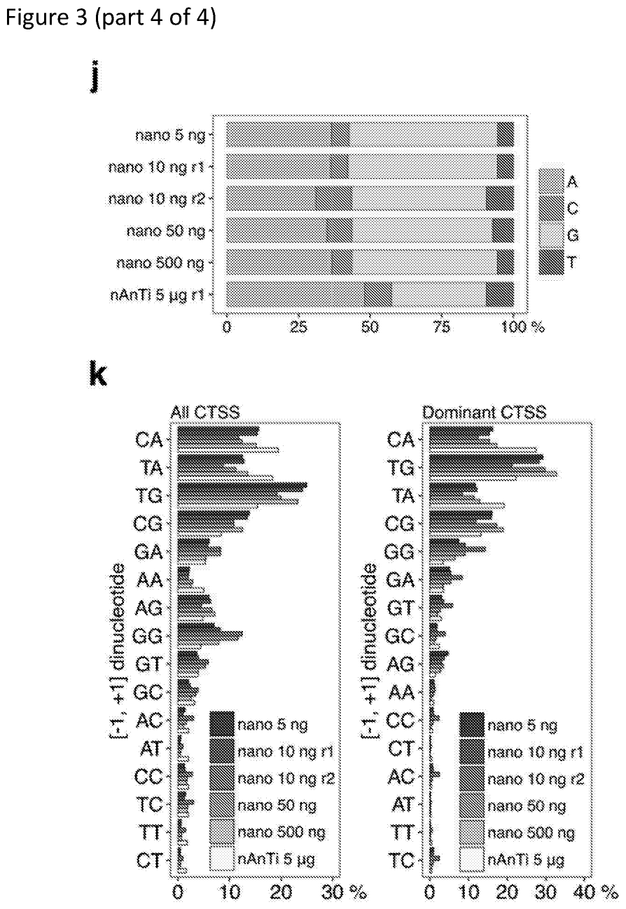

nanoCAGE was compared to nAnT-iCAGE. A nanoCAGE titration test was carried out using S. cerevisiae total RNA (5-500 ng) and compared the obtained libraries with the reference nAnT-iCAGE library. CTSSs identified in nanoCAGE libraries were poorly correlated (Pearson correlation 0.5-0.6) with the nAnT-iCAGE library, irrespective of the total RNA used (FIG. 3a-e and FIG. 16b). Despite reduced similarity with nAnT-iCAGE, nanoCAGE libraries appeared reproducible (FIG. 3f). An example genome browser view also reveals significant differences in CTSS profiles between nanoCAGE and nAnT-iCAGE libraries (FIG. 3g). NanoCAGE systematically failed to capture all CTSSs identified with nAnT-iCAGE. In contrast, the SLIC-CAGE library derived from only 5 ng of total RNA accurately recapitulates the nAnT-iCAGE TSS profile shown in the same genomic region (FIG. 3g as FIG. 1e).

The tag clusters identified in each nanoCAGE library was investigated and showed that approximately 85% were indeed in expected promoter regions (FIG. 3h, and FIG. 16e). The cluster overlap is highly similar to the reference nAnT-iCAGE library in all nanoCAGE libraries, independent of the amount of total RNA used. Therefore, nanoCAGE does not capture the full complexity of promoter TSS usage but its specificity for promoters is not diminished.

To inspect the complexity of nanoCAGE libraries, the tag cluster IQ-widths was compared with the reference nAnT-iCAGE library (FIG. 3i and FIG. 17c). An increase in the number of sharper tag clusters is observed at 1-50 ng of total input RNA. The IQ-width distributions show that nanoCAGE systematically produces lower-complexity libraries compared to nAnT-iCAGE and SLIC-CAGE. This result agrees well with the consistently lower number of unique CTSSs identified in nanoCAGE libraries compared to nAnT-iCAGE libraries (FIG. 13a).

Nucleotide composition of nanoCAGE-identified robust CTSSs revealed a strong preference for G-containing CTSS (FIG. 3j). This is specific to nanoCAGE libraries compared to nAnT-iCAGE and also independent of the total input RNA. This observed G-preference is not an artefact caused by the extra C added complementary to the cap structure at the 5′end of cDNA during reverse transcription, as that is common to all CAGE protocols and corrected in all datasets using the processing step in the Bioconductor package CAGEr²⁵. Lastly, to check if in nanoCAGE more than one G is added during reverse transcription, the 5′end Gs flagged as a mismatch in the alignment were counted, indicating that the amount of two consecutive mismatches was not significant (FIG. 13B).

The composition of [−1,+1] initiator dinucleotides revealed a severe depletion in identified CA and TA initiator, with the corresponding increase in G-containing initiators (TG and CG), in comparison with the reference nAnT-iCAGE dataset (FIG. 3k, left panel, Supplementary FIG. 2k). To assess the most robust CTSSs, the same analysis was repeated using only the dominant CTSSs in each tag cluster (FIG. 3k, right panel, Supplementary FIG. 2n) and the lack of CA and TA initiators was equally apparent. This property of nanoCAGE makes it unsuitable for the determination of dominant CTSSs and details of promoter architecture at base pair resolution.

To exclude the effects of CTSSs located in non-promoter regions and to assess if CTSS identification depends on expression levels, tag clusters were divided according to their genomic location or expression values (division into four expression quartiles per each library) and repeated the analysis (FIGS. 20 and 21). Since a similar pattern (depletion of CA and TA initiators) was observed irrespective of the genomic location or expression level, these results suggest that the nanoCAGE bias is a consequence of the template switching property of reverse transcriptase, which is known to be sequence dependent, and is expected to prefer capped RNA that starts with G27.

Signal ratios of individual CTSSs identified in each nanoCAGE library and the reference nAnT-iCAGE were analysed (ratio of TPM values, FIG. 22 left panels), and CTSSs not identified in nanoCAGE (FIG. 22. middle panels) similarly as described for SLIC-CAGE (see above). This analysis reveals that there are no position specific biases in nanoCAGE and that the biases are primarily caused by nucleotide composition of the capped RNA 5′ends. Further, it accentuates the inability of nanoCAGE to capture dominant CTSSs identified with the reference nAnT-iCAGE, even with higher amounts of starting material, compared to SLIC-CAGE (FIG. 22f-h vs FIG. 18a-h).

### Example 4—Use of SLIC-CAGE in Uncovering Promoter Architecture

The dominant CTSS provides a structural reference point for the alignment of promoter sequences and thus facilitates the discovery of promoter-specific sequence features. High-quality data is necessary for the accurate identification of the dominant TSS within a tag cluster or promoter region. Sharp promoters, defined by small IQ-widths, are typically defined by a fixed distance from a core promoter motif, such as a TATA-box or TATA-like element at −30 position 28 upstream of the TSS, or by DPE motif at +28 to +3229 in Drosophila. Broader promoters, featuring multiple CTSS positions, are enriched for GC content and CpG island overlap 7 in vertebrates. Lower complexity libraries have an increased number of artificially sharp tag clusters (FIG. 2f), due to sparse CTSS identification. Although the identified CTSSs in lower-complexity SLIC-CAGE libraries are canonical, association of sequence features may be obscured by artificially sharp tag clusters. To address this, the promoter architecture for known promoter features in E14 mouse cell lines was investigated using SLIC-CAGE from 5 to 100 ng of total RNA.

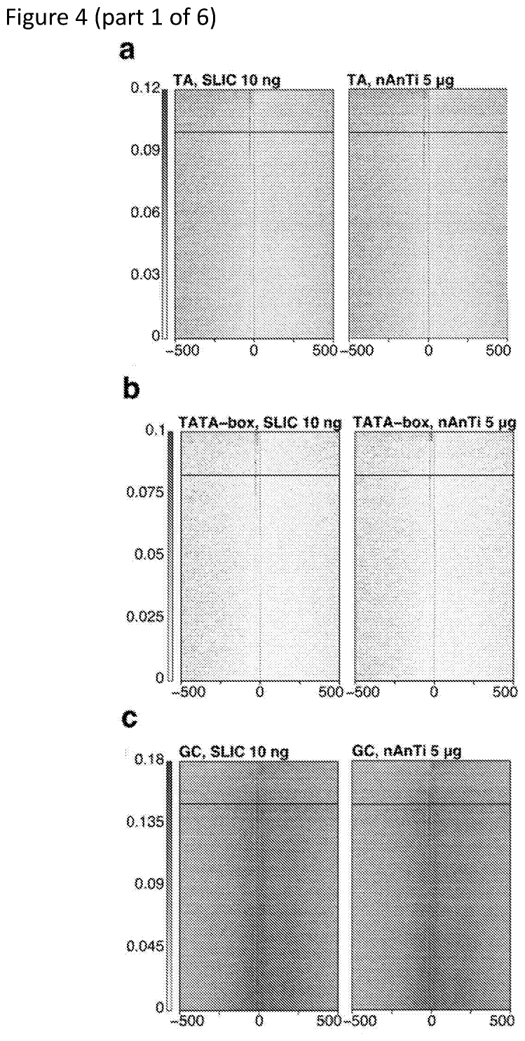

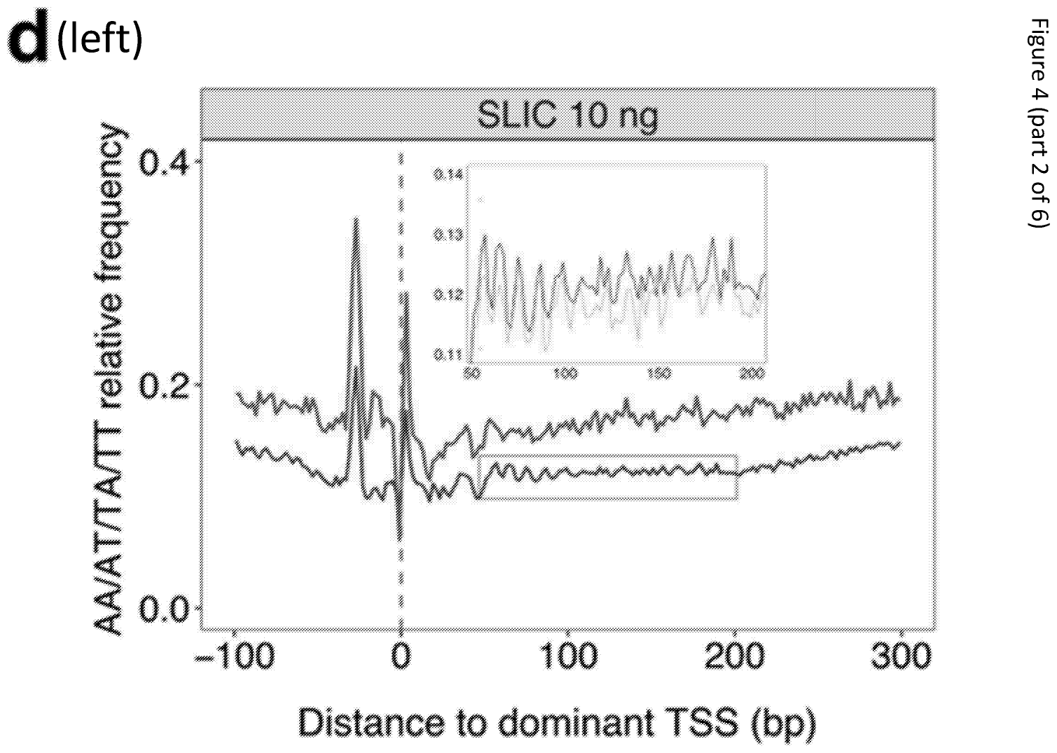

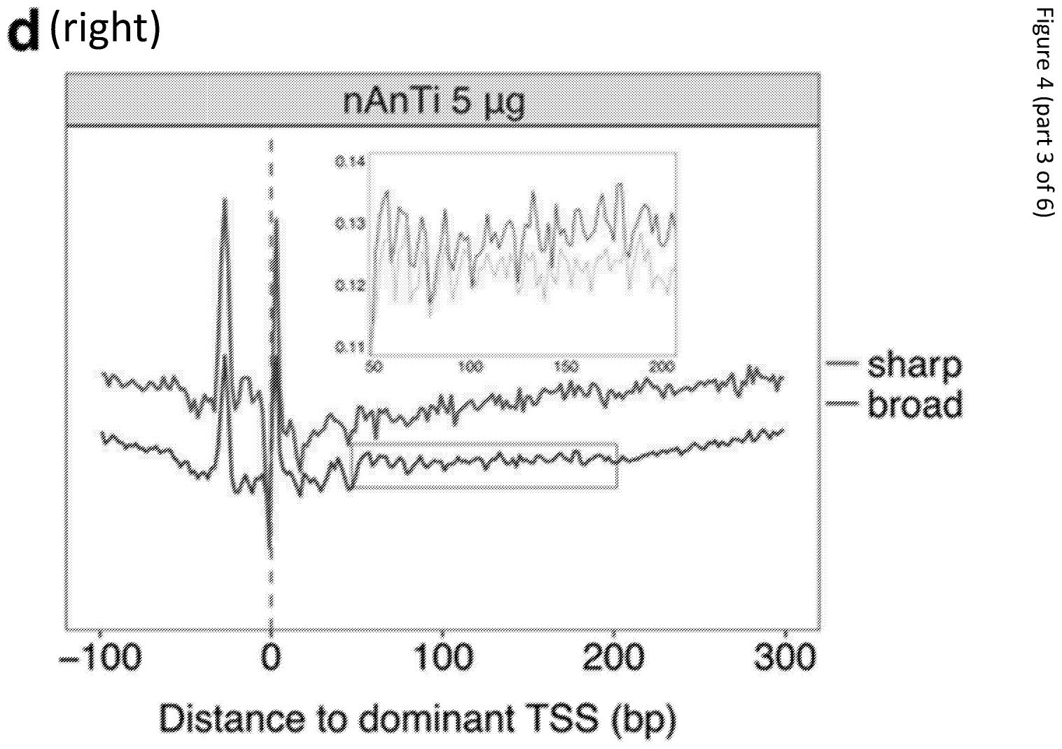

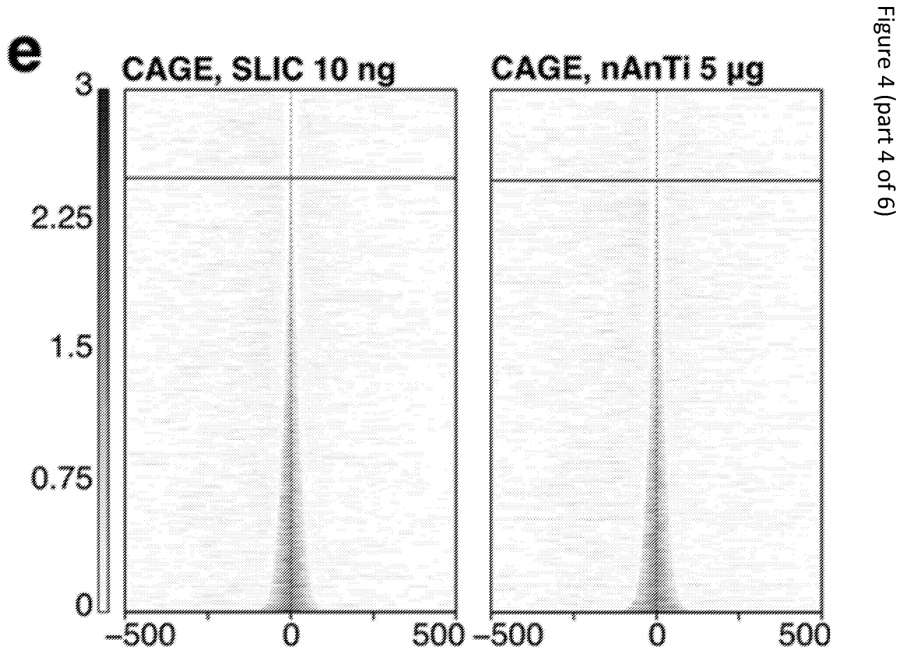

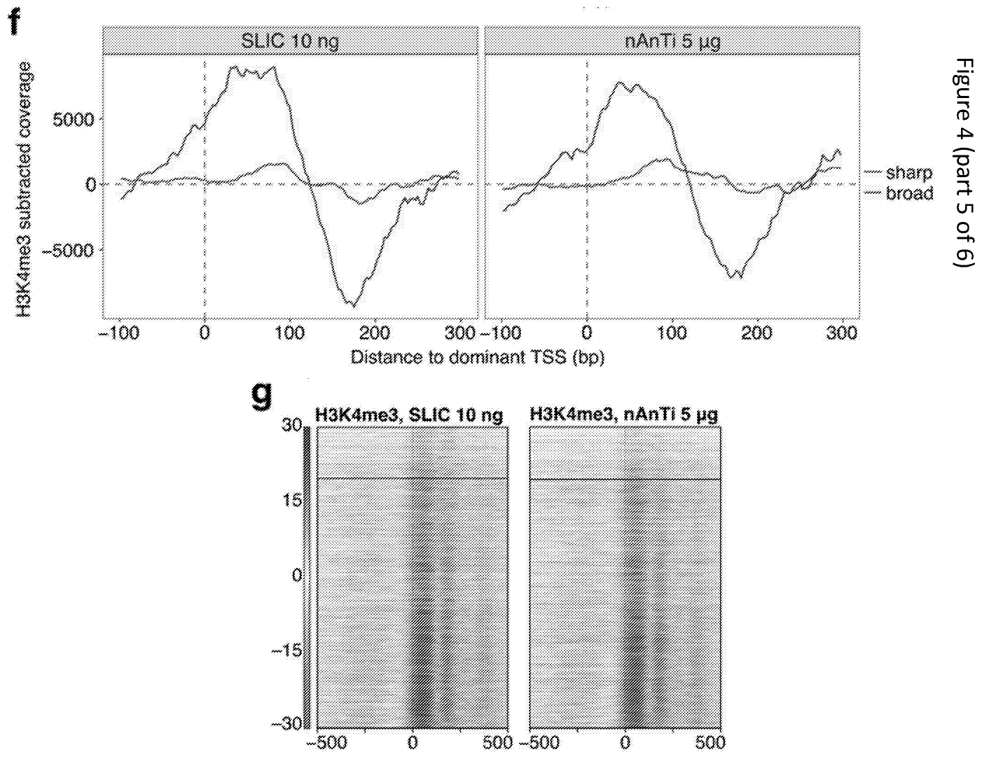

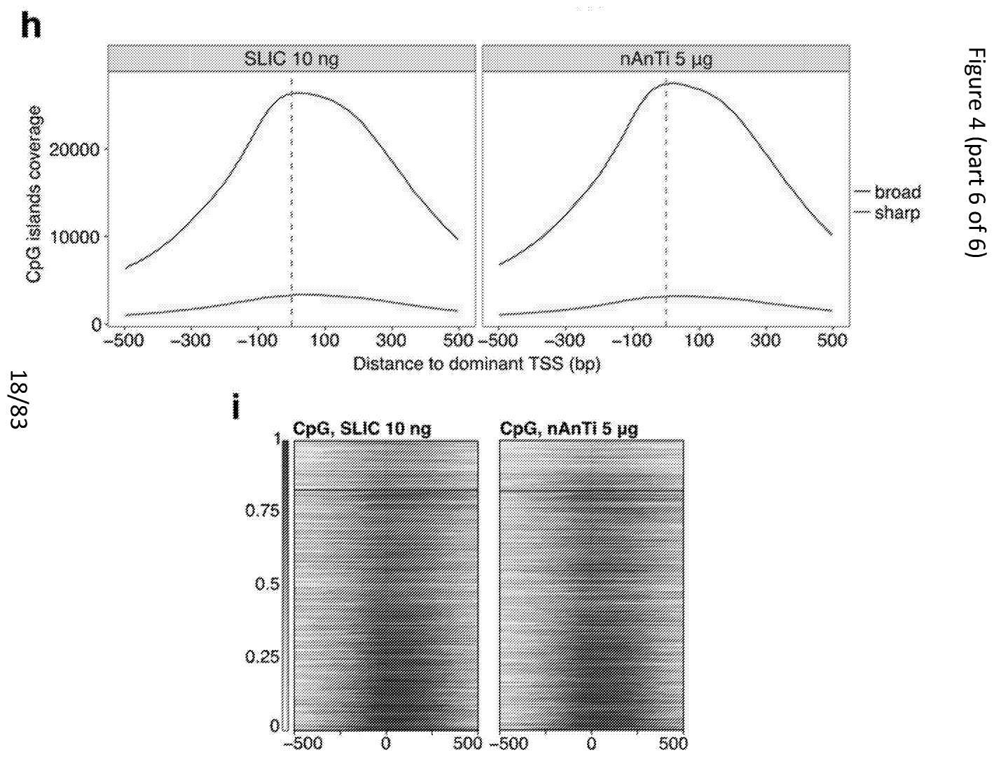

The presence of a TA dinucleotide around the −30 positions for all TSS identified by SLIC-CAGE for both 5 and 10 ng of input RNA. The TSSs were ordered by IQ-width of their corresponding tag cluster and extended to include 1 kb DNA sequence up- and downstream. The TA frequency is depicted in a heatmap in FIG. 4a for promoters ordered from sharp to broad for 10 ng of RNA and clearly recapitulates the patterns visible in the reference nAnT-iCAGE library. As expected, the sharpest tag clusters in libraries produced from 5 ng of total RNA have a weaker TA signal (FIG. 24a, TA heatmap), as these are likely artificially sharp and not the canonical sharp promoters. A similar result is observed for enrichment of the canonical TATA-box element, the 10 ng library recapitulated the reference nAnT-iCAGE library whereas the 5 ng library shows a weaker enrichment (FIG. 4b and FIG. 23c,d).

A GC-enrichment in the region between the dominant TSS and 250 nucleotides downstream of it indicates positioning of the +1 nucleosomes and is expected to be highly localized in broad promoters. This feature is again recapitulated by the 10 ng RNA input library (FIG. 4c, FIG. 24)^{3, 5, 7}. Furthermore, rotational positioning of the +1 nucleosomes are associated with VWV periodicity (AA/AT/TA/TT dinucleotides) lined up with the dominant TSS. VWV dinucleotide density was examined separately for sharp and broad promoters identified by SLIC-CAGE and the reference nAnT-iCAGE library (FIG. 4d). A strong 10.5 nucleotides periodicity of VWV dinucleotides downstream of the dominant TSS was observed in SLIC-CAGE libraries derived from 10 ng of M. musculus total RNA and corresponded to the phasing observed with the reference nAnT-iCAGE library (FIG. 4d and FIG. 23b). This can only be observed across promoters if the dominant TSS is accurately identified and therefore it reflects the quality of the libraries. To confirm that VWV dinucleotide periodicity reflects +1 nucleosome positioning in broad promoters, we assessed H3K4me3 data downloaded from ENCODE (FIGS. 4f and g, FIG. 24 H3K4me3 heatmap). H3K4me3 subtracted coverage reflects the well-positioned +1 nucleosome broad promoters (FIG. 4f,g) and localizes with VWV periodicity specific for broad promoters (FIG. 4d). These results are in agreement with previously identified nucleosome positioning preferences³⁰.

As a final validation of SLIC-CAGE promoters, CpG island density was assessed separately in sharp and broad promoters (FIG. 4h,l, FIG. 24). A higher density of CpG islands was observed in SLIC-CAGE broad promoters, which corresponds to nAnT-iCAGE broad promoters and agrees with the expected association of broad promoters and CpG islands.7, 24 These results demonstrate the utility of SLIC-CAGE libraries derived from nanogram-scale samples in promoter architecture discovery, alongside the gold standard nAnT-iCAGE libraries.

### Example 5—Discussion

The inventors have developed SLIC-CAGE, an unbiased cap-trapper based CAGE protocol optimized for promoterome discovery from as little as 5-10 ng of isolated total RNA (approximately 10³ cells). SLIC-CAGE libraries are of equivalent quality and complexity as nAnT-iCAGE libraries derived from 500-1000-fold more material (5 μg of total RNA, approximately 10⁶ cells). SLIC-CAGE extends the nAnT-iCAGE protocol through addition of the degradable carrier to the target RNA material of limited availability. Since the best CAGE protocol is not amenable to downscaling, the idea behind the carrier is to increase the amount of material to permit highly specific cap-trapper based purification of target RNA polymerase II transcripts and to minimize material loss in many protocol steps.

The carrier was designed to have a similar size distribution and fraction of capped molecules as the total cellular RNA, to effectively saturate non-specific adsorption sites on all surfaces and matrices used throughout the protocol. In the final stage of SLIC-CAGE, the carrier molecules are selectively degraded using homing endonucleases, while the intact target library is amplified and sequenced.

SLIC-CAGE has been shown herein to have superior sensitivity, resolution and absence of bias to the only other low-input CAGE technology, nanoCAGE, which relies on template switching during the cDNA synthesis¹⁵. Although the amount of starting material is significantly reduced, the lowest input limit for nanoCAGE is 50 ng of total RNA which may require up to 30 PCR cycles¹⁶. The performances of SLIC-CAGE and nanocage were directly compared in titration tests and demonstrated that: 1) higher complexity libraries are achieved with significantly lower input: SLIC-CAGE requires 5-10 ng, while nanoCAGE requires 50 ng of total RNA; 2) nanoCAGE does not recapitulate the complexity of the nAnT-iCAGE libraries even with the highest recommended amount of RNA (500 ng), in comparison SLIC-CAGE captures the full complexity when 5-10 ng are used; 3) nanoCAGE preferentially captures G-starting capped mRNAs, while SLIC-CAGE does not have 5′mRNA nucleotide dependent biases; 4) Biases in nanoCAGE libraries are independent of the total RNA amount used, and inherent to the template switching step.

Importantly, with the carrier approach to minimize the target sample loss, SLIC-CAGE protocol requires less PCR amplification cycles—15-18 cycles for 10-1 ng of total RNA as input. This is advantageous as smaller number of PCR cycles reduce amplification biases and the fraction of observed duplicate reads. Although, nanoCAGE takes advantage of unique molecular identifiers to remove PCR duplicates, in our experience, synthesis of truly random UMI is problematic and subject to variability, thereby obscuring its use.

A different carrier-based approach has recently been applied to down-scale chromatin-precipitation based methods—favoured amplification recovery via protection ChIP-seq (FARP-ChIP-seq)³¹. FARP-ChIP-seq relies on a designed biotinylated synthetic DNA carrier, mixed with chromatin of interest prior to ChIP-seq library preparation. Amplification of the synthetic DNA carrier is prevented using specific blocker oligonucleotides. These blocker oligonucleotides are: 1) complementary to the biotin-DNA carrier; 2) carry phosphorothioate modification of the first three nucleotides at the 5′end for resistance to exonuclease activity of the polymerase; 3) carry a 3′end 3-carbon spacer to inhibit extension by PCR. The blocker strategy can achieve a 99% reduction in amplification of the biotin-DNA, which if applied instead of our degradable carrier would leave much more carrier to sequence (starting SLIC-CAGE with 1 ng of total RNA and 5 μg of the carrier, 27% of the carrier is left in the final library, which is more than a 10000-fold reduction, FIG. 11). While also being more costly and time consuming, this approach could be combined with selective degradation of the SLIC-CAGE carrier when near-complete removal of carrier is required thereby increasing sequencing depth to allow higher complexity libraries from even lower input amounts.

SLIC-CAGE is expected to prove to be invaluable for in-depth and high-resolution promoter analysis of rare cell types, including early embryonic developmental stages or embryonic tissue from a wide range of model organisms, which has so far been inaccessible to the method. With its low material requirement (5-10 ng of total RNA), SLIC-CAGE can also be applied on isolated nascent RNA, to provide an unbiased promoterome with high positional and temporal resolution. Lastly, as bidirectional capped RNA is a signature feature of active enhancers4, deeply sequenced SLIC-CAGE libraries can be used to identify active enhancers in rare cell types. The principle of the degradable carrier can also easily be extended to other protocols where the required amount of RNA or DNA is limiting.

### Example 6—Methods

### Preparation of the Carrier RNA Molecules

DNA template (1 kb length) for preparation of the carrier by in vitro transcription was synthesized and cloned into pJ241 plasmid (service by DNA 2.0, FIGS. 5 and 15) to produce the carrier plasmid. The template encompasses the gene that serves as the carrier, embedded with restriction sites for I-SceI and I-CeuI to allow degradation in the final steps of the library preparation. The templates for in vitro transcription were prepared by PCR amplification using the unique forward primer (PCR_GN5_f1, FIG. 6) which introduces the T7 promoter followed by five random nucleotides, and the reverse primer which determines the total length of the carrier template and introduces six random nucleotides at the 3′end (FIG. 6 PCR_N6_r1-r10).

The PCR reaction to produce the carrier templates was composed of 0.2 ng p1-1 carrier plasmid, 1 μM primers (each), 0.02 U μl-1 Phusion High-Fidelity DNA Polymerase (Thermo Fisher Scientific) and 0.2 mM dNTP in 1×Phusion HF Buffer (final concentrations). The cycling conditions are presented in FIG. 7. Produced carrier templates (lengths 1034-386 nucleotides) were gel-purified to remove non-specific products.

Carrier RNA was in vitro transcribed using HiScribe™ T7 High Yield RNA Synthesis Kit (NEB) according to manufacturer's instructions, and purified using RNeasy Mini kit (Qiagen). A portion of carrier RNAs was capped using Vaccinia Capping System and purified using RNeasy Mini kit (Qiagen). The capping efficiency was estimated using RNA 5′Polyphosphatase and Terminator™ 5′-Phosphate-Dependent Exonuclease, as only uncapped RNAs are dephosphorylated and degraded, while capped RNA's are protected. Several carrier combinations were tested in SLIC-CAGE (FIGS. 7B and 8A) and the final carrier used in SLIC-CAGE was comprised of 90% uncapped carrier and 10% capped carrier, both of varying length (FIG. 8A).

### SLIC-CAGE Library Preparation

For the standard cap analysis of gene expression, the latest nAnT-iCAGE protocol was followed¹⁴. In the SLIC-CAGE variant, the carrier was mixed with the RNA of interest, to the total amount of 5 μg, e.g. 10 ng of RNA of interest were mixed with 4990 ng of carrier mix and subjected to reverse transcription as in the nAnT-iCAGE protocol¹⁴. Further library preparation steps were followed as described in Murata et al 2014¹⁴ with several exceptions: 1) samples were pooled only prior to sequencing, to allow individual quality control steps; 2) samples were never completely dried using the centrifugal concentrator and then redissolved as in nAnT-iCAGE, instead the leftover volume was monitored to avoid complete drying and adjusted with water to achieve the required volume; 3) After the final AMPure purification in the nAnT-iCAGE protocol, each sample was concentrated using the centrifugal concentrator, and its volume adjusted to 15 μl, out of which 1 μl was used for quality control on the Agilent Bioanalyzer HS DNA chip.

Steps regarding degradation of the carrier in SLIC-CAGE libraries are schematically presented in FIG. 25.

To degrade the carrier, 14 μl of sample was mixed with I-SceI (5 U) and I-CeuI (5 U) in 1×CutSmart buffer (NEB) and incubated at 37° C. for 3 h. The enzymes were heat inactivated at 65° C. for 20 min, and the samples purified using AMPure XP beads (1.8×AMPure XP volume per reaction volume, as described in Murata et al 14). The libraries were eluted in 42 μl of water and concentrated to 20 μl using the centrifugal concentrator. A qPCR control was then performed to determine the suitable number of PCR cycles for library amplification and assess the amount of the leftover carrier. The primers designed to amplify the whole library are complementary to 5′ and 3′ linker regions, while the primers used to selectively amplify just the carrier are complementary to the 5′end of the carrier (common to all carrier molecules) and the 3′linker (common to all molecules in the library, see Supplementary Table 6 for primer sequences). qPCR reactions were performed using KAPA SYBR FAST qPCR kit using 1 μl of the sample and 0.1 μM primers (final concentration), in 10 μl total volume using PCR cycle conditions presented in the FIG. 8C.

The number of cycles for PCR amplification of the library corresponded to the Ct value obtained with the primers that amplify the whole library (adapter_f1 and adapter_r1, FIG. 8B). PCR amplification of the library was then performed using KAPA HiFi HS ReadyMix, with 0.1 μM primers (adapter_f1 and adapter_r1, FIG. 8B) and 18 μl of sample in a total volume of 100 μl. The cycling programme is presented in the FIG. 8D and the final number of cycles used to amplify the libraries in FIG. 9. Amplified samples were purified using AMPure XP beads (1.8×volume ratio of the beads to the sample), eluted with 42 μl of water and concentrated using centrifugal concentrator to 14 μl.

A second round of carrier degradation was then performed as described for the first round. The samples were purified using AMPure XP beads (stringent 1:1 AMPure XP to sample volume ratio to exclude primer dimers and short fragments), eluted with 42 μl of water and concentrated to 12 μl using centrifugal concentrator. The combination of 1st round of carrier degradation followed by PCR amplification, AMPure XP purification and the 2nd round of carrier degradation is necessary to avoid substantial sample loss that leads to low-complexity libraries.

Each sample was then individually assessed for fragment size distribution using an HS DNA chip (Bioanalyzer, Agilent). If short fragments were present in the library (<300 nucleotides, see Supplement FIG. 12), another round of size selection was performed using a stringed volume ratio of AMPure XP beads to the sample—0.8×(volume of each sample was prior to purification adjusted with water to 30 μl). The samples were eluted in 42 μl of water and concentrated to 12 μl using centrifugal concentrator. Fragment size distribution was again checked using an HS DNA chip (Bioanalyzer, Agilent), to ensure removal of the short fragments.

Finally, the amount of leftover carrier was estimated using qPCR as described above after the 1st round of carrier degradation. The expected Ct in qPCR using adapter_f1 and adapter_r1 is 12-13 or 23-30 using carrier_f1 and adapter_r1 primer pairs (FIG. 8B) when the starting total RNA amount is 100-1 ng.

The libraries were sequenced on MiSeq (S. cerevisiae) or HiSeq2500 (M. musculus) Illumina platforms in single-end 50 base-pair mode (Genomics Facility, MRC, LMS),

### NanoCAGE Library Preparation

S. cerevisiae nanoCAGE libraries were prepared as described in the latest protocol version by Poulain et al 2017¹⁶. Briefly, 5, 10, 25, 50 or 500 ng of S. cerevisiae total RNA was reversely transcribed in the presence of corresponding template switching oligonucleotides (FIG. 14) followed by AMPure purification. One 500 ng replicate was pre-treated with exonuclease to test if rRNA removal has any effects on the quality of the final library.

The number of PCR-cycles for semi-suppressive PCR was determined by qPCR as described in Poulain et al 2017 (FIG. 9). Samples were AMPure purified after amplification, and the concentration of each sample determined using Picogreen.

2 ng of each sample were pooled prior tagmentation and 0.5 ng of the pool was used in tagmentation. The sample was AMPure purified and quantified using Picogreen prior to MiSeq sequencing in single-end 50 base-pair mode (Genomics Facility, MRC, LMS).

Processing of CAGE Tags: nAnT-iCAGE, SLIC-CAGE or nanoCAGE

Sequenced CAGE tags (50 nucleotides) were mapped to a reference S. cerevisiae genome (sacCer3 assembly) or M. musculus genome (mm10 assembly) using Bowtie232 with default parameters that allow zero mismatches per seed sequence (default 20 nucleotides). Sequenced nanoCAGE libraries were trimmed prior to mapping to remove the linker and UMI region (15 nucleotides from the 5′end were trimmed).

Only uniquely mapped reads were used in downstream analysis within R graphical and statistical computing environment (http://www.R-project.org/) using Bioconductor packages (http://www.bioconductor.org/) and custom scripts. The mapped reads were sorted and imported into R as bam files using CAGEr25. The additional G nucleotide at the 5′end of the reads, if added through template free activity of the reverse transcriptase, was resolved within CAGEr's standard workflow designed to remove G's that do not map to the genome: 1) if the first nucleotide is G and a mismatch, i.e. it does not map to the genome, it is removed from the read; 2) if the first nucleotide is G and it matches, it is retained or removed according to the percentage of mismatched G.

All unique 5′ends represent CAGE tag-supported TSSs (CTSSs), and the number of tags within each CTSS represents expression levels. Raw tag counts were normalized using a referent power-law distribution to a total of 106 tags, resulting in normalized tags per million (TPMs) 33.

Clustering of CTSSs into Tag Clusters

CTSSs that pass the threshold of 1 TPM in at least one of the samples were clustered using a distance-based method implemented in the CAGEr package with a maximum allowed distance of 20 nucleotides between the neighbouring CTSS.

For each tag cluster, a cumulative distribution of signal was calculated and the boundaries of the tag cluster calculated using the 10th and 90th percentile of its signal. The distance between these boundaries represents the interquantile width of a tag cluster.

### Genomic Locations of Tag Clusters

Tag clusters were annotated with their corresponding genomic locations using the ChIPseeker package 34. In S. cerevisiae libraries, promoters were defined as 1 kb windows centred on Ensembl 35 annotated transcriptions start sites (annotations imported from SGD) and in M. musculus libraries, promoters were defined as <=1 kb or 1-3 kb from the UCSC annotated transcription start site.

### Nucleotide and Dinucleotide Composition of CTSSs

CTSSs from each library were filtered prior to analysis to include only CTSS with at least 1 TPM. In each library the number of A, C, G or T-containing CTSS was counted, divided by the total number of filtered CTSSs and converted to a percentage. The same analysis was performed using only dominant TSS (identified using the CAGEr package as a CTSS with highest expression within a tag cluster).

For dinucleotide analysis, identified filtered CTSSs were extended to include one upstream nucleotide ([−1, +1] dinucleotides where +1 represents the identified CTSS) and the same analysis as described above repeated for 16 possible dinucleotides.

Dinucleotide Pattern Analysis in M. musculus Libraries

Heatmaps Bioconductor package (Perry M (18). heatmaps: Flexible Heatmaps for Functional Genomics and Sequence Features. R package version 1.2.0) was used to visualize dinucleotide patterns (TA and GC) across sequences centred on the dominant TSS. Sequences were ordered by interquantile width of the containing tag cluster, with the sharpest on top and broadest tag cluster on the bottom of the heatmap. Raw data with the exact matching for TA or GC was smoothed prior to plotting using kernel smoothing within the heatmaps package. Each heatmap was divided into two sections based on tag cluster's IQ-widths. Empirical boundary (Supplementary FIG. 9a) was set to separate sharp (IQ-width <=3 nucleotides) and broad (IQ-width >3) tag clusters identified in M. musculus libraries. The horizontal line/boundary was implemented using heatmaps options to partition heatmaps/rows of an image.

TATA-Box Motif Analysis in M. musculus Libraries

SeqPattern package was used to scan the sequences for the occurrence of the TATA-box motif using a threshold of 80th percentile match to the TATA-box PWM (imported from the seqpattern package). We further smoothed the obtained results using the kernel smoothing (heatmaps package) and plotted the results with sequences ordered by interquantile width of the containing tag cluster (sharpest on top and broadest on bottom of the tag cluster) and centred on the dominant TSS. The horizontal line in each heatmap represents the empirical boundary that separates sharp (IQ-width <=3) and broad tag clusters (IQ-width >3).

TATA-box metaplots (average signal/profile) were produced separately for sharp and broad tag clusters (see definition above). Seqpattern was used for scanning sequences using TATA-box PWM to identify 80% matches. The results were converted to the average signal using the heatmaps package with a 2 nucleotides bin size. The final data was plotted using the ggplot2 package 36.

Nucleosome Positioning Signal in in M. musculus Libraries—WW Periodicity

WW dinucleotide (AA/AT/TA/TT) occurrence (average relative signal) was obtained using the heatmaps package separately for sharp and broad tag clusters (see definition above). A 2 nucleotides bin size was used and the sequences were centred on the dominant TSS. As a control for the importance of centring the sequences on the dominant TSS, WW dinucleotide (AA/AT/TA/TT) occurrence was obtained as an average relative signal from sequences where each sequence is centred on a randomly chosen CTSS within a tag cluster. The final data was plotted using the ggplot2 package36.

H3K4Me3 Signal Around M. musculus Tag Clusters

H3K4me3 data for E14 cell line, mapped to mm10 was downloaded from ENCODE experiment ENCSR000CGO. Bam files for two replicates (accession numbers ENCFF997CAQ and ENCFF425ZMWO) were merged using samtools 37 and the merged bam file was imported to R environment using the rtracklayer package 38

H3K4me3 coverage was calculated separately for reads mapping to minus or plus strand and minus strand reads subtracted from plus strand reads to get the subtracted H3K4me3 coverage.

Subtracted H3K4me3 coverage was visualized using heatmaps package centred on the dominant TSSs with sequences ordered by IQ-width of the containing tag clusters (sharpest on top, and broadest at the bottom of the heatmap). Each heatmap was divided into two sections based on tag cluster's IQ-widths as described above.

H3K4me3 coverage metaplots were produced separately for sharp and broad tag clusters (see definition above, only strongly supported dominant CTSSs with at least 5 TPM were used) using heatmaps package with a 3 nucleotides bin size The final data was plotted using the ggplot2 package36.

M. musculus Tag Cluster Overlap with CpG Islands

The CpG island track for mm10 was downloaded from the UCSC Genome Browser. Overlap with M. musculus tag clusters was visualized as a coverage heatmap using heatmaps package, centred on the dominant TSS with sequences ordered by IQ-width of the containing tag clusters (sharpest on top, and broadest at the bottom of the heatmap). Each heatmap was divided into two sections based on tag cluster's IQ-widths as described above.

CpG coverage metaplots were produced separately for sharp and broad tag clusters (see definition above) using heatmaps package with a 3 nucleotides bin size. The final data was plotted using the ggplot2 package36.

### Code and Data Availability

All custom scripts are available upon request and data is accessible at: https://drive.google.com/open?id=1T4ZL7JFnaWITUHD7LaLvu74r_IZYG60N

### Example 7

### New Method for CAGE

The new method for CAGE is intended to improve sequencing efficiency on Illumina sequencing instruments (Illumina HiSeq2500) and shorten the protocol. Fewer protocol steps should lead to higher complexity libraries achieved with lower amount of total cellular RNA (currently the protocol is optimised to work with 5-10 ng, and optimisations may allow use of 1-2 ng).

### Changes in Protocol Steps:

Average fragment length in the final SLIC-CAGE libraries is 800 nucleotides. Clustering of fragments on Illumina sequencers is more efficient for shorter fragments (standard Illumina sequencing libraries tend to have fragment size 200-500 nucleotides), therefore larger fragments typically lower sequencing efficiency. To improve sequencing quality of the SLIC-CAGE libraries, a tagmentation step (Illumina Nextera XT kit) is incorporated. The kit relies on transposition of barcode sequences randomly into DNA in a “cut and paste” reaction. This random insertion efficiently fragments the DNA and at the same time adds the sequences required for PCR amplification and sequencing. Incorporation of the tagmentation step after the SLIC-CAGE protocol is performed has been tested and analysis of the resultant libraries is underway.

SLIC-CAGE methodology relies on nAnT-iCAGE protocol steps. However, by including tagmentation to decrease the size of the DNA fragments, the sequence necessary for sequencing and PCR amplification is added at the same time. Therefore, it is expected that nAnTi-CAGE steps which include ligation of the 3′linker, and treatment with the USER enzyme will be unnecessary. Replacement of the 3′linker ligation in the nAnT-iCAGE protocol with the tagmentation step is currently being investigated. As the carrier is still present in the libraries, and tagmentation involves also the PCR amplification step, the following conditions are being optimised and tested:

### A)

1. cleavage of the cDNA by a transposon and tagging of the double-stranded cDNA, for example tagmentation;
2. PCR amplification
3. Degradation of the carrier nucleic acid according to the invention or the nucleic acid of the compositions according to the invention;
4. Purification of the DNA fragments, for example by Solid Phase Reversible Immobilization-based paramagnetic beads, for example AMPure beads or RNAclean XP; or

### B)

1. Degradation of the nucleic acid according to the invention or the nucleic acid of the compositions according to the invention;
2. Purification of the DNA fragments, for example by Solid Phase Reversible Immobilization-based paramagnetic beads, for example AMPure beads or RNAclean XP; or
3. cleavage of the cDNA by a transposon and tagging of the double-stranded cDNA, for example tagmentation;
4. PCR amplification; or

### C)

1. Degradation of the carrier nucleic acid according to the invention or the nucleic acid of the compositions according to the invention;
2. Purification of the DNA fragments, for example by Solid Phase Reversible Immobilization-based paramagnetic beads, for example AMPure beads or RNAclean XP;
3. PCR amplification
4. (optional 2nd round of degradation of the carrier nucleic acid according to the invention or the nucleic acid of the compositions according to the invention)
5. Cleavage of the cDNA by a transposon and tagging of the double-stranded cDNA, for example tagmentation.

Libraries will be tested by qPCR for the presence of the carrier, and if necessary, 2^{nd} round of carrier degradation and size-selection will be performed.

1. A nucleic acid comprising at least a first sequence and a second sequence wherein at least one of the first or second sequence is an endonuclease recognition sequence or is capable of acting as an endonuclease recognition sequence when converted into a corresponding double-stranded DNA form,
wherein the length of the endonuclease recognition sequence is at least 15 nucleotides.

2. The nucleic acid according to claim 1 wherein both the first and the second sequence is an endonuclease recognition sequence or is capable of acting as an endonuclease recognition sequence when converted into a corresponding double-stranded DNA form,
and wherein the length of the first and second endonuclease recognition sequence is at least 15 nucleotides.

3. The nucleic acid according to any of claim 1 or 2 wherein the nucleic acid comprises at least three sequences wherein each sequence is an endonuclease recognition sequence or is capable of acting as an endonuclease recognition sequence when converted into a corresponding double-stranded DNA form, and wherein the length of each endonuclease recognition sequence is at least 15 nucleotides, optionally wherein the nucleic acid comprises at least 4, optionally at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 350, at least 400, at least 450, at least 500, at least 550, at least 600, at least 650, or at least 700 sequences wherein each sequence is an endonuclease recognition sequence or is capable of acting as an endonuclease recognition sequence when converted into a corresponding double-stranded DNA form, and wherein the length of each endonuclease recognition sequence is at least 15 nucleotides.

4. The nucleic acid according to any of claims 1-3 wherein the length of the nucleic acid is 10 kb or less, optionally less than 9.5 kb, optionally less than 9.0 kb, optionally less than 8.5 kb, optionally less than 8.0 kb, optionally less than 7.5 kb, optionally less than 7.0 kb, optionally less than 6.5 kb, optionally less than 6.0 kb, optionally less than 5.5 kb, optionally less than 5.0 kb, optionally less than 4.5 kb, optionally less than 4.0 kb, optionally less than 3.5 kb, optionally less than 3.0 kb, optionally less than 2.5 kb, optionally less than 2.0 kb, optionally less than 1.5 kb, optionally less than 1.25 kb, optionally less than 1.0 kb, optionally less than 900 nucleotides, optionally less than 800 nucleotides, optionally less than 700 nucleotides, optionally less than 600 nucleotides, optionally less than 500 nucleotides, optionally less than 400 nucleotides, optionally less than 300 nucleotides, optionally less than 200 nucleotides, optionally less than 100 nucleotides.

5. The nucleic acid according to any of claims 1-4 wherein the length of the nucleic acid is between 100 nucleotides and 10 kb in length, optionally between 200 nucleotides and 9 kb, optionally between 300 nucleotides and 8 kb, optionally between 400 nucleotides and 7 kb, optionally between 500 nucleotides and 6 kb, optionally between 600 nucleotides and 5 kb, optionally between 700 nucleotides and 4 kb, optionally between 800 nucleotides and 3 kb, optionally between 900 nucleotides and 2 kb, optionally 1 kb.

6. The nucleic acid according to any of claims 1-5 wherein at least the first or the second sequence that is an endonuclease recognition sequence or that is capable of acting as an endonuclease recognition sequence when converted into a corresponding double-stranded DNA form is at least 16 nucleotides or greater in length, optionally at least 17 nucleotides or greater in length, optionally at least 18 nucleotides or greater in length, optionally at least 19 nucleotides or greater in length, optionally at least 20 nucleotides or greater in length, optionally at least 21 nucleotides or greater in length, optionally at least 22 nucleotides or greater in length, optionally at least 23 nucleotides or greater in length, optionally at least 24 nucleotides or greater in length, optionally at least 25 nucleotides or greater in length, optionally at least 26 nucleotides or greater in length, optionally at least 27 nucleotides or greater in length, optionally at least 28 nucleotides or greater in length, optionally at least 29 nucleotides or greater in length, optionally at least 30 nucleotides or greater in length, optionally at least 31 nucleotides or greater in length, optionally at least 32 nucleotides or greater in length, optionally at least 33 nucleotides or greater in length, optionally at least 34 nucleotides or greater in length, optionally at least 35 nucleotides or greater in length,
optionally wherein both of the first sequence and a second sequence that are endonuclease recognition sequences or that are capable of acting as endonuclease recognition sequences when converted into a corresponding double-stranded DNA form are at least 16 nucleotides or greater in length, optionally at least 17 nucleotides or greater in length, optionally at least 18 nucleotides or greater in length, optionally at least 19 nucleotides or greater in length, optionally at least 20 nucleotides or greater in length, optionally at least 21 nucleotides or greater in length, optionally at least 22 nucleotides or greater in length, optionally at least 23 nucleotides or greater in length, optionally at least 24 nucleotides or greater in length, optionally at least 25 nucleotides or greater in length, optionally at least 26 nucleotides or greater in length, optionally at least 27 nucleotides or greater in length, optionally at least 28 nucleotides or greater in length, optionally at least 29 nucleotides or greater in length, optionally at least 30 nucleotides or greater in length, optionally at least 31 nucleotides or greater in length, optionally at least 32 nucleotides or greater in length, optionally at least 33 nucleotides or greater in length, optionally at least 34 nucleotides or greater in length, optionally at least 35 nucleotides or greater in length,
optionally wherein any number of or all of the sequences that are endonuclease recognition sequences or that are capable of acting as endonuclease recognition sequences when converted into a corresponding double-stranded DNA form are at least 16 nucleotides or greater in length, optionally at least 17 nucleotides or greater in length, optionally at least 18 nucleotides or greater in length, optionally at least 19 nucleotides or greater in length, optionally at least 20 nucleotides or greater in length, optionally at least 21 nucleotides or greater in length, optionally at least 22 nucleotides or greater in length, optionally at least 23 nucleotides or greater in length, optionally at least 24 nucleotides or greater in length, optionally at least 25 nucleotides or greater in length, optionally at least 26 nucleotides or greater in length, optionally at least 27 nucleotides or greater in length, optionally at least 28 nucleotides or greater in length, optionally at least 29 nucleotides or greater in length, optionally at least 30 nucleotides or greater in length, optionally at least 31 nucleotides or greater in length, optionally at least 32 nucleotides or greater in length, optionally at least 33 nucleotides or greater in length, optionally at least 34 nucleotides or greater in length, optionally at least 35 nucleotides or greater in length.

7. The nucleic acid according to any of claims 1-6 wherein the first and second sequence that are endonuclease recognition sequences or that are capable of acting as endonuclease recognition sequences when converted into a corresponding double-stranded DNA form are the same length, optionally wherein any number of or all of the sequences that are endonuclease recognition sequences or that are capable of acting as endonuclease recognition sequences when converted into a corresponding double-stranded DNA form are the same length.

8. The nucleic acid according to any of claims 1-6 wherein the first and second sequence that are endonuclease recognition sequences or that are capable of acting as endonuclease recognition sequences when converted into a corresponding double-stranded DNA form are of different lengths, optionally wherein any number of or all of the sequences that are endonuclease recognition sequences or that are capable of acting as endonuclease recognition sequences when converted into a corresponding double-stranded DNA form are of different lengths.

9. The nucleic acid according to any of claims 1-8 wherein the nucleic acid comprises sequences that are endonuclease recognition sequences or that are capable of acting as endonuclease recognition sequences when converted into a corresponding double-stranded DNA form of at least three different lengths length, optionally at least four different lengths, optionally at least five different lengths, optionally at least six different lengths, optionally at least seven different lengths, optionally at least eight different lengths, optionally at least nine different lengths, optionally at least 10 different lengths.

10. The nucleic acid according to any of claims 1-9 wherein the first sequence and the second sequence that are endonuclease recognition sequences or that are capable of acting as endonuclease recognition sequences when converted into a corresponding double-stranded DNA form are identical to one another, optionally wherein any number of or all of the sequences that are endonuclease recognition sequences or that are capable of acting as endonuclease recognition sequences when converted into a corresponding double-stranded DNA form are identical to one another.

11. The nucleic acid according to any of claims 1-9 wherein the first sequence and the second sequence that are endonuclease recognition sequences or that are capable of acting as endonuclease recognition sequences when converted into a corresponding double-stranded DNA form are different to one another, optionally wherein any number of or all of the sequences that are endonuclease recognition sequences or that are capable of acting as endonuclease recognition sequences when converted into a corresponding double-stranded DNA form are different to one another.

12. The nucleic acid according to any of claims 1-11 wherein the sequences that are endonuclease recognition sequences or that are capable of acting as endonuclease recognition sequences when converted into a corresponding double-stranded DNA form are recognition sequences for the same endonuclease, optionally wherein any number of or all of the sequences that are endonuclease recognition sequences or that are capable of acting as endonuclease recognition sequences when converted into a corresponding double-stranded DNA form are recognition sequences for the same endonucleases.

13. The nucleic acid according to any of claims 1-12 wherein the sequences that are endonuclease recognition sequences or that are capable of acting as endonuclease recognition sequences when converted into a corresponding double-stranded DNA form are recognition sequences for different endonucleases, optionally wherein any number of or all of the sequences that are endonuclease recognition sequences or that are capable of acting as endonuclease recognition sequences when converted into a corresponding double-stranded DNA form are recognition sequences for different endonucleases.

14. The nucleic acid according to any of claims 1-13 wherein the sequences that are endonuclease recognition sequences or that are capable of acting as endonuclease recognition sequences when converted into a corresponding double-stranded DNA form are recognition sequences for at least three different endonucleases, optionally at least four different endonucleases, optionally at least five different endonucleases, optionally at least six different endonucleases, optionally at least seven different endonucleases, optionally at least eight different endonucleases, optionally at least nine different endonucleases, optionally at least 10 different endonucleases.

15. The nucleic acid according to any of claims 1-14 wherein at least one of the sequences that are endonuclease recognition sequences or that are capable of acting as endonuclease recognition sequences when converted into a corresponding double-stranded DNA form are recognition sequences for a homing endonuclease,
optionally wherein two of the sequences that are endonuclease recognition sequences or that are capable of acting as endonuclease recognition sequences when converted into a corresponding double-stranded DNA form are recognition sequences for homing endonuclease enzymes,
optionally wherein all of the sequences that are endonuclease recognition sequences or that are capable of acting as endonuclease recognition sequences when converted into a corresponding double-stranded DNA form are recognition sequences for homing endonuclease enzymes.

16. The nucleic acid according to any of claims 1-15 wherein the first and/or the second sequence is repeated within the nucleic acid, optionally wherein the first and/or the second sequence occurs at least twice within the nucleic acid, optionally at least three times, optionally at least four times, optionally at least five times, optionally at least 6 times, optionally at least 7 times, optionally at least 8 times, optionally at least 9 times, optionally at least 10 times, optionally at least 12 times, optionally at least 14 times, optionally at least 16 times, optionally at least 18 times, optionally at least 20 times, optionally at least 25 times, optionally at least 30 times, optionally at least 35 times, optionally at least 40 times, optionally at least 45 times, optionally at least 50 times, optionally at least 55 times, optionally at least 60 times, optionally at least 65 times, optionally at least 70 times, optionally at least 75 times, optionally at least 80 times, optionally at least 85 times, optionally at least 90 times, optionally at least 95 times, optionally at least 100 times, optionally at least 110 times, optionally at least 120 times, optionally at least 130 times, optionally at least 140 times, optionally at least 150 times, optionally at least 160 times, optionally at least 170 times, optionally at least 180 times, optionally at least 190 times, optionally at least 200 times.

17. The nucleic acid according to any of claims 1-16 wherein both the first sequence and the second sequence are repeated in the nucleic acid in alternating fashion, optionally wherein where the nucleic acid comprises at least three sequences that are endonuclease recognition sequences or are capable of acting as endonuclease recognition sequences when converted into a corresponding double-stranded DNA form each of the sequences are repeated in an alternating fashion.

18. The nucleic acid according to any of claims 1-17 wherein the first sequence and the second sequence are arranged such that cleavage of the first sequence results in the production of nucleic acid fragments that are all less than 200 nucleotides, optionally less than 180 nucleotides, optionally less than 160 nucleotides, optionally less than 140 nucleotides, optionally less than 120 nucleotides, optionally less than 100 nucleotides, optionally less than 80 nucleotides, optionally less than 60 nucleotides, optionally less than 40 nucleotides, optionally less than 20 nucleotides.

19. The nucleic acid according to any of claims 1-18 wherein the first sequence and the second sequence are arranged such that cleavage of the second sequence results in the production of nucleic acid fragments that are all less than 200 nucleotides, optionally less than 180 nucleotides, optionally less than 160 nucleotides, optionally less than 140 nucleotides, optionally less than 120 nucleotides, optionally less than 100 nucleotides, optionally less than 80 nucleotides, optionally less than 60 nucleotides, optionally less than 40 nucleotides, optionally less than 20 nucleotides.

20. The nucleic acid according to any of claims 1-19 wherein the first sequence and the second sequence are arranged such that cleavage of the first sequence and the second sequence results in the production of nucleic acid fragments that are all less than 200 nucleotides, optionally less than 180 nucleotides, optionally less than 160 nucleotides, optionally less than 140 nucleotides, optionally less than 120 nucleotides, optionally less than 100 nucleotides, optionally less than 80 nucleotides, optionally less than 60 nucleotides, optionally less than 40 nucleotides, optionally less than 20 nucleotides.

21. The nucleic acid according to any of claims 1-20 wherein cleavage of the first and/or second sequence results in the production of nucleic acid fragments that are all less than 80 nucleotides, optionally less than 70 nucleotides.

22. The nucleic acid according to any of claims 1-21 wherein cleavage of the first and/or second sequence results in the production of nucleic acid fragments that can be removed by Solid Phase Reversible Immobilization, optionally Solid Phase Reversible Immobilization-based paramagnetic beads, optionally AMPure beads or RNAclean XP beads.

23. The nucleic acid according to any of claims 1-22 wherein the first and/or second sequences that are endonuclease recognition sequences or that are capable of acting as endonuclease recognition sequences when converted into a corresponding double-stranded DNA form are recognition sequences for a homing endonuclease wherein the homing endonuclease is selected from the group consisting of:
BneMS4ORFIP, F-CphI, F-EcoT3I, F-EcoT5I, F-EcoT5II, F-EcoT5IV, F-PhiU5I, F-SceI, F-SceII, F-TevI, F-TevII, F-TevIII, F-TevIV, H-DreI, H-DreI, I-AabMI, I-AchMI, I-AniI, I-ApeKI, I-BanI, I-BasI, I-BmoI, I-Bth0305I, I-BthII, 1-BthORFAP, I-CeuI, I-ChuI, I-CmoeI, I-CpaI, I-CpaII, I-CpaMI, I-CreI, I-CreII, I-CsmI, I-CvuI, I-DdiI, I-DmoI, I-GpeMI, I-GpiI, I-GzeI, I-GzeII, I-HjeMI, I-HmuI, I-HmuII, 1-LlaI, I-LtrI, I-LtrWI, I-MpeMI, I-MsoI, I-NanI, I-NfiI, I-NitI, I-NjaI, I-OmiII, I-OnuI, I-PakI, I-PanMI, I-PfoP3I, I-PnoMI, I-PogTE7I, I-PorI, I-PpoI, I-ScaI, I-SceI, I-SceII, I-SceIII, I-SceIV, I-SceV, I-SceVI, I-SceVII, I-SecIII, I-SmaMI, I-SpomI, I-SscMI, I-Ssp6803I, I-TevI, I-TevII. I-TevIII. I-TsII. I-TsIWI, I-Tsp061I, I-TwoI, I-Vdi141I, -AvaI, PI-BciPI, PI-HvoWI, PI-MtuI, PI-PabI, PI-PabII, PI-PfuI, PI-PfuII, PI-PkoI, PI-PkoII, PI-PspI, PI-PspI, PI-ScaI, PI-SceI, PI-TfuI, PI-TfuII, PI-ThyI, PI-TliI, PI-TliII, PI-TmaI, PI-TmaKI, PI-ZbaI.

24. The nucleic acid according to any of claims 1-23 wherein the first and/or second sequence that are endonuclease recognition sequences or that are capable of acting as endonuclease recognition sequences when converted into a corresponding double-stranded DNA form are recognition sequences is selected from the group consisting of: SEQ ID NO: 1-142, optionally SEQ ID NO: 1 and/or SEQ ID NO: 2.

25. The nucleic acid according to any of claims 1-24 wherein the nucleic acid comprises one or more modifications, optionally a modification selected from the group consisting of biotinylation.

26. The nucleic acid according to any of claims 1-25 wherein the nucleic acid is an RNA nucleic acid, optionally wherein the RNA comprises a 5′ cap.

27. The nucleic acid according to any of claims 1-25 wherein where the nucleic acid is an RNA the RNA does not comprise a 5′ cap.

28. The nucleic acid according to any of claims 1-25 wherein the nucleic acid is a DNA, optionally a double-stranded DNA nucleic acid.

29. A vector comprising the nucleic acid according to any of claims 1-28.

30. A vector comprising a sequence that is capable of being transcribed into an RNA transcript, wherein the transcript comprises a nucleic acid according to any of claims 1-28.

31. A cell comprising a nucleic acid according to any of claims 1-28 or a vector according to any of claim 29 or 30, optionally wherein the cell is:
a) a prokaryotic cell, optionally a bacterial cell, optionally an E. coli cell, a Bacillus subtilis cell, a Bacillus megaterium cell, a Vibrio natriegens cell, or a Pseudomonas fluorescens cell; or
b) a eukaryotic cell, optionally
a yeast cell, optionally Pichia pastoris or Saccharomyces cerevisiae;
an insect cell, optionally a baculovirus infected insect cell; or
a mammalian cell, optionally a baculovirus infected mammalian cell, a HEK293 cell, a HeLa cell, or CHO cells.

32. A composition comprising at least one nucleic acid according to any of claims 1-28.

33. A composition comprising at least two nucleic acids according to any of claims 1-28 wherein the at least two nucleic acids are of different sequence to one another, optionally comprising at least 3 different nucleic acids wherein the at least 3 different nucleic acids are of different sequence to one another, optionally comprising at least 4 different nucleic acids wherein the at least 4 different nucleic acids are of different sequence to one another, optionally comprising at least 5 different nucleic acids wherein the at least 5 different nucleic acids are of different sequence to one another, optionally comprising at least 6 different nucleic acids wherein the at least 6 different nucleic acids are of different sequence to one another, optionally comprising at least 7 different nucleic acids wherein the at least 7 different nucleic acids are of different sequence to one another, optionally comprising at least 8 different nucleic acids wherein the at least 8 different nucleic acids are of different sequence to one another, optionally comprising at least 9 different nucleic acids wherein the at least 9 different nucleic acids are of different sequence to one another, optionally comprising at least 10 different nucleic acids wherein the at least 10 different nucleic acids are of different sequence to one another.

34. A composition comprising at least two nucleic acids according to any of claims 1-28 wherein the at least 2 nucleic acids are of different length, optionally at least 3 nucleic acids according to any of claims 1-24 wherein the at least 3 nucleic acids are of different lengths, optionally at least 4 nucleic acids according to any of claims 1-24 wherein the at least 4 nucleic acids are of different lengths, optionally at least 5 nucleic acids according to any of claims 1-24 wherein the at least 5 nucleic acids are of different lengths, optionally at least 6 nucleic acids according to any of claims 1-24 wherein the at least 6 nucleic acids are of different lengths, optionally at least 7 nucleic acids according to any of claims 1-24 wherein the at least 7 nucleic acids are of different lengths, optionally at least 8 nucleic acids according to any of claims 1-24 wherein the at least 8 nucleic acids are of different lengths, optionally at least 9 nucleic acids according to any of claims 1-24 wherein the at least 9 nucleic acids are of different lengths, optionally at least 10 nucleic acids according to any of claims 1-24 wherein the at least 10 nucleic acids are of different lengths.

35. The composition according to any of claims 32-34 wherein the composition comprises 10 different nucleic acids according to any of claims 1-28 wherein each of the 10 nucleic acids is of a different length, optionally wherein the composition comprises a nucleic acid according to any of claims 1-28 of each of the following lengths:
i) between 1000 nucleotides and 1200 nucleotides, optionally 1100 nucleotides, optionally 1034 nucleotides;
ii) between 900 nucleotides and 1000 nucleotides, optionally between 920 nucleotides and 980 nucleotides, optionally between 960 nucleotides and 970 nucleotides, optionally 966 nucleotides;
iii) between 850 nucleotides and 900 nucleotides, optionally between 860 nucleotides and 890 nucleotides, optionally 889 nucleotides;
iv) between 800 nucleotides and 850 nucleotides, optionally between 810 nucleotides and 840 nucleotides, optionally between 820 nucleotides and 830 nucleotides, optionally 821 nucleotides;
v) between 700 nucleotides and 800 nucleotides, optionally between 720 nucleotides and 780 nucleotides, optionally between 740 nucleotides and 760 nucleotides, optionally 744 nucleotides;
vi) between 650 nucleotides and 700 nucleotides, optionally between 660 nucleotides and 690 nucleotides, optionally between 670 nucleotides and 680 nucleotides, optionally 676 nucleotides;
vii) between 550 nucleotides and 650 nucleotides, optionally between 560 nucleotides and 640 nucleotides, optionally between 570 nucleotides and 630 nucleotides, optionally between 580 nucleotides and 620 nucleotides, optionally between 590 nucleotides and 610 nucleotides, optionally 599 nucleotides or 600 nucleotides;
viii) between 500 nucleotides and 550 nucleotides, optionally between 510 nucleotides and 540 nucleotides, optionally between 520 nucleotides and 530 nucleotides, optionally 531 nucleotides;
ix) between 400 nucleotides and 500 nucleotides, optionally between 410 nucleotides and 490 nucleotides, optionally between 420 nucleotides and 480 nucleotides, optionally between 430 nucleotides and 470 nucleotides, optionally between 440 nucleotides and 460 nucleotides, optionally 450 nucleotides or 454 nucleotides; and
x) between 300 nucleotides and 400 nucleotides, optionally between 310 nucleotides and 390 nucleotides, optionally between 320 nucleotides and 380 nucleotides, optionally between 330 nucleotides and 370 nucleotides, optionally between 340 nucleotides and 360 nucleotides, optionally 350 nucleotides or 386 nucleotides;
optionally wherein the composition comprises at least 10 different nucleic acids according to any of claims 1-25 wherein the nucleic acids are 1034 nucleotides in length, 966 nucleotides in length, 889 nucleotides in length, 821 nucleotides in length, 744 nucleotides in length, 676 nucleotides in length, 599 nucleotides in length, 531 nucleotides in length, 454 nucleotides in length, and 386 nucleotides in length.

36. The composition according to any of claims 32-35 wherein the composition comprises capped RNA nucleic acids according to any of claims 1-28, optionally comprises capped and uncapped RNA nucleic acids according to any of claims 1-28.

37. The composition according to any of claims 32-36 wherein the range of sizes of the nucleic acids according to any of claims 1-28 are similar to the range of sizes of RNA or DNA nucleic acids in a sample.

38. The composition according to any of claims 32-37 wherein the percentage of RNA nucleic acids that comprise a 5′ cap is similar to the percentage of capped RNA nucleic acids in a sample.

39. The composition according to any of claims 32-38 wherein the composition comprises RNA nucleic acids according to any of 1-28, wherein at least 5% of the RNA nucleic acids comprises a 5′ cap, optionally at least 10% of the RNA nucleic acids comprises a 5′ cap, optionally at least 20% of the RNA nucleic acids comprises a 5′ cap, optionally at least 30% of the RNA nucleic acids comprises a 5′ cap, optionally at least 40% of the RNA nucleic acids comprises a 5′ cap, optionally at least 50% of the RNA nucleic acids comprises a 5′ cap, optionally at least 60% of the RNA nucleic acids comprises a 5′ cap, optionally at least 70% of the RNA nucleic acids comprises a 5′ cap, optionally at least 80% of the RNA nucleic acids comprises a 5′ cap, optionally at least 90% of the RNA nucleic acids comprises a 5′ cap, optionally 100% of the RNA nucleic acids comprises a 5′ cap.

40. A kit comprising:
at least one nucleic acid according to any of claims 1-28, optionally comprising at least two nucleic acids according to any of claims 1-28, optionally comprising at least 3 nucleic acids according to any of claims 1-28, optionally comprising at least 4 nucleic acids according to any of claims 1-28, optionally comprising at least 5 nucleic acids according to any of claims 1-28, optionally comprising at least 6 nucleic acids according to any of claims 1-28, optionally comprising at least 7 nucleic acids according to any of claims 1-28, optionally comprising at least 8 nucleic acids according to any of claims 1-28, optionally comprising at least 9 nucleic acids according to any of claims 1-28, optionally comprising at least 10 nucleic acids according to any of claims 1-28; and/or
at least one vector according to any of claims 29 and 30; and/or
at least one cell according to claim 31; and/or
at least one composition according to any of claims 32-39.

41. The kit according to claim 40 wherein the kit comprises at least one nucleic acid according to any of claims 1-28 that is a capped RNA and at least one nucleic acid according to any of claims 1-28 that is an uncapped RNA.

42. The kit according to any of claims 40 and 41 wherein the kit comprises at least two nucleic acids according to any of claims 1-28 wherein the at least two nucleic acids are of different lengths, optionally at least 3 nucleic acids according to any of claims 1-28 wherein the at least 3 nucleic acids are of different lengths, optionally at least 4 nucleic acids according to any of claims 1-28 wherein the at least 4 nucleic acids are of different lengths, optionally at least 5 nucleic acids according to any of claims 1-28 wherein the at least 5 nucleic acids are of different lengths, optionally at least 6 nucleic acids according to any of claims 1-28 wherein the at least 6 nucleic acids are of different lengths, optionally at least 7 nucleic acids according to any of claims 1-28 wherein the at least 7 nucleic acids are of different lengths, optionally at least 8 nucleic acids according to any of claims 1-28 wherein the at least 8 nucleic acids are of different lengths, optionally at least 9 nucleic acids according to any of claims 1-28 wherein the at least 9 nucleic acids are of different lengths, optionally at least 10 nucleic acids according to any of claims 1-28 wherein the at least 10 nucleic acids are of different lengths.

43. The kit according to any of claims 40-42 wherein where the at least one nucleic acid according to any of claims 1-28 is an RNA nucleic acid, the kit comprises the RNA nucleic acid in a capped and uncapped form,
optionally wherein the kit comprises 2 different RNA nucleic acids according to any one of claims 1-28, each in both a capped and uncapped form,
optionally wherein the kit comprises 3 different RNA nucleic acids according to any one of claims 1-28, each in both a capped and uncapped form,
optionally wherein the kit comprises 4 different RNA nucleic acids according to any one of claims 1-28, each in both a capped and uncapped form,
optionally wherein the kit comprises 5 different RNA nucleic acids according to any one of claims 1-28, each in both a capped and uncapped form,
optionally wherein the kit comprises 6 different RNA nucleic acids according to any one of claims 1-28, each in both a capped and uncapped form,
optionally wherein the kit comprises 7 different RNA nucleic acids according to any one of claims 1-28, each in both a capped and uncapped form,
optionally wherein the kit comprises 8 different RNA nucleic acids according to any one of claims 1-28, each in both a capped and uncapped form,
optionally wherein the kit comprises 9 different RNA nucleic acids according to any one of claims 1-28, each in both a capped and uncapped form,
optionally wherein the kit comprises 10 different RNA nucleic acids according to any one of claims 1-28, each in both a capped and uncapped form.

44. The kit according to any of claims 40-43 wherein the kit comprises one or more endonuclease enzymes, optionally wherein at least one of the endonuclease enzymes is a homing endonuclease enzyme,
and wherein the at least one endonuclease enzyme recognises at least one of the sequences that are endonuclease recognition sequences or that are capable of acting as endonuclease recognition sequences when converted into a corresponding double-stranded DNA form.

45. A method of isolating nucleic acid from a sample wherein the method comprises the use of any one or more of:
the nucleic acids according to any of claims 1-28;
the vectors according to any one of claim 29 or 30;
the cell according to claim 31;
the compositions according claim 32-40; and/or
the kits according to claims 41-44.

46. A method for improving the yield of nucleic acid obtained from a sample, wherein the method comprises the use of any one or more of:
the nucleic acids according to any of claims 1-28;
the vectors according to any one of claim 29 or 30;
the cell according to claim 31;
the compositions according claim 32-40; and/or
the kits according to claims 41-44.

47. The method of any of claim 45 or 46 wherein the sample is a small sample, optionally wherein the sample comprises:
0.1 ng to 500 ng of nucleic acids; and/or
Less than 5000 cells, optionally less than 4000 cells, optionally less than 2000 cells, optionally less than 1000 cells, optionally less than 800 cells, optionally less than 600 cells, optionally less than 400 cells, optionally less than 200 cells, optionally around 100 cells or less.

48. The method of any of claims 45-47 wherein the sample is selected from the group consisting of:
a sample from an embryo; a sample of oocytes, FACS sorted cells, rare cell types, small biopsies, primordial germ cells, and samples of an embryo in the early embryonic developmental stages.

49. The method according to any of claims 45-48 wherein the method further comprises contacting the nucleic acid according to any of claims 1-28 or the composition according to any of claims 32-40 with at least one endonuclease, optionally at least one homing endonuclease.

50. A method for isolating nucleic acid that will be sequenced wherein the method comprises the use of any one or more of:
the nucleic acids according to any of claims 1-28;
the vectors according to any one of claim 29 or 30;
the cell according to claim 31;
the compositions according claim 32-40; and/or
the kits according to claims 41-44.

51. A method for sequencing a nucleic acid wherein the method comprises the use of any one or more of:
the nucleic acids according to any of claims 1-28;
the vectors according to any one of claim 29 or 30;
the cell according to claim 31;
the compositions according claim 32-40; and/or
the kits according to claims 41-44.

52. A method for cap analysis of gene expression (CAGE) wherein the method comprises the use of any one or more of:
the nucleic acids according to any of claims 1-28 optionally wherein the nucleic acid according to any of claims 1-28 is an RNA nucleic acid;
the vectors according to any one of claim 29 or 30;
the cell according to claim 31;
the compositions according claim 32-40; and/or
the kits according to claims 41-44.

53. The method according to claim 52 wherein the method further comprises contacting the nucleic acid according to any one or more of claims 1-28 or the composition according to any of claims 32-40 with at least one endonuclease, optionally at least one homing endonuclease.

54. The method according to claim 53 wherein said contacting occurs following reverse transcription of the RNA to DNA.

55. A method for cap analysis of gene expression (CAGE) wherein the method comprises cleavage of the cDNA by a transposon and tagging of the double-stranded cDNA, optionally wherein the method comprises tagmentation.

56. The method according to claim 55 wherein the method comprises the use of any one or more of:
the nucleic acids according to any of claims 1-28 optionally wherein the nucleic acid according to any of claims 1-28 is an RNA nucleic acid; and/or
the compositions according claim 32-40;

57. The method according to any of claim 55 or 56 wherein the method does not comprise a 3′ linker ligation reaction and/or does not comprise uracil specific excision reagent (USER) treatment.

58. The method according to any of claims 56 and 57 wherein the method comprises the following steps in the following order:
A)
1. cleavage of the cDNA by a transposon and tagging of the double-stranded cDNA, for example tagmentation;
2. PCR amplification
3. Degradation of the carrier nucleic acid according to the invention or the nucleic acid of the compositions according to the invention;
4. Purification of the DNA fragments, for example by Solid Phase Reversible Immobilization-based paramagnetic beads, for example AMPure beads or RNAclean XP; or
B)
1. Degradation of the nucleic acid according to the invention or the nucleic acid of the compositions according to the invention;
2. Purification of the DNA fragments, for example by Solid Phase Reversible Immobilization-based paramagnetic beads, for example AMPure beads or RNAclean XP; or
3. cleavage of the cDNA by a transposon and tagging of the double-stranded cDNA, for example tagmentation;
4. PCR amplification; or
C)
1. Degradation of the carrier nucleic acid according to the invention or the nucleic acid of the compositions according to the invention;
2. Purification of the DNA fragments, for example by Solid Phase Reversible Immobilization-based paramagnetic beads, for example AMPure beads or RNAclean XP;
3. PCR amplification
4. (optional 2nd round of degradation of the carrier nucleic acid according to the invention or the nucleic acid of the compositions according to the invention)
5. Cleavage of the cDNA by a transposon and tagging of the double-stranded cDNA, for example tagmentation.

59. A method for assessing gene promoters and/or transcription start sites, the method comprising:
a) providing a sample of target nucleic acid; and
b) mixing the sample of nucleic acid with a nucleic acid according to any of claims 1-28 or a composition according to any of claims 32-40.

60. A method of generating a nucleic acid library, optionally a cDNA library, wherein the method comprises the use of any one or more of:
the nucleic acids according to any of claims 1-28 optionally wherein the nucleic acid according to any of claims 1-28 is an RNA nucleic acid;
the vectors according to any one of claim 29 or 30;
the cell according to claim 31;
the compositions according claim 32-40;
the kits according to claims 41-44; and/or
the methods according to claims 45-59.

61. A method of diagnosis wherein the method comprises the use of any one or more of:
the nucleic acids according to any of claims 1-28 optionally wherein the nucleic acid according to any of claims 1-28 is an RNA nucleic acid;
the vectors according to any one of claim 29 or 30;
the cell according to claim 31;
the compositions according claim 32-40;
the kits according to claims 41-44; and/or
the methods according to claims 45-60.

62. A method of chromatin immunoprecipitation (ChIP), ChIP-seq, or FARP-ChIP-seq wherein the method comprises the use of any one or more of:
the nucleic acids according to any of claims 1-28 optionally wherein the nucleic acid according to any of claims 1-28 is an RNA nucleic acid;
the vectors according to any one of claim 29 or 30;
the cell according to claim 31;
the compositions according claim 32-40;
the kits according to claims 41-44; and/or
the methods according to claims 45-60.

63. The method of any preceding claim wherein the method also comprises the use of an oligo blocker of carrier amplification during PCR amplification.
